(19) United States
(12) Patent Application Publication
(10) Pub. No.: US 2004/0228866 A1
(43) Pub. Date: Nov. 18, 2004

(54) Suppressor genes

(75) Inventor: Xin Lu, London (GB)

Correspondence Address: KLARQUIST SPARKMAN, LLP, 121 SW SALMON STREET, SUITE 1600, PORTLAND, OR, 97204, US

(73) Assignee: Ludwig Institute for Cancer Research

(21) Appl. No.: 10/819,095

(22) Filed: Apr. 5, 2004

Related U.S. Application Data
(63) Continuation-in-part of application No. 10/343,649, filed on Sep. 4, 2003.
Application No. 10/343,649 is a 371 of international application No. PCT/GB01/03524, filed on Aug. 6, 2001.

(30) Foreign Application Priority Data
Aug. 4, 2000 (GB) 0019018.1
Dec. 8, 2000 (GB) 0029996.6
May 26, 2001 (GB) 0112890.9

(51) Int. Cl. A61K 39/395; A61K 48/00; A61K 38/17; C07H 21/04

(52) U.S. Cl. 424/155.1; 514/12; 514/44; 536/23.2

(57) ABSTRACT

The disclosure relates to the identification of a new member of a family of tumour suppressor genes (apoptosis stimulating proteins of p53, ASPP's) which encode polypeptides capable of modulating the activity of p53, p63 and p73, and polypeptides capable of modulating the activity of a tumour suppressor polypeptide.

### CROSS REFERENCE TO RELATED APPLICATIONS

This is a continuation-in-part of U.S. application Ser. No. 10/343,649 filed Feb. 3, 2003, which is a §371 U.S. National stage of PCT/GB01/03524 filed Aug. 6, 2001, which claims priority to Great Britain Application No: 0019018.1 filed Aug. 4, 2000, Great Britain Application No: 0029996.6 filed Dec. 8, 2000, and Great Britain Application No: 0112890.9 filed May 26, 2001, all incorporated by reference in their entirety.

### FIELD

This application relates to members of a family of tumour suppressor genes, Apoptosis Stimulating Proteins of p53 (ASPP), which encode polypeptides capable of modulating the activity of p53, p63, and p73, and methods of their use to increase apoptosis, for example to treat a tumor.

### BACKGROUND

Tumour suppressor genes encode proteins that reduce or inhibit cell growth or division. Mutations in tumour suppressor genes result in abnormal cell-cycle progression whereby the normal cell-cycle check points which arrest the cell-cycle, for example when DNA is damaged, are ignored and damaged cells divide uncontrollably. The products of tumour suppressor genes function in all parts of the cell (such as the cell surface, cytoplasm, and nucleus) to prevent the passage of damaged cells through the cell-cycle (G1, S, G2, M and cytokinesis).

Several tumour suppressor genes have been identified. For example, mutations in the retinoblastoma gene (Rb) are linked to cancers in the bone (osteocarcoma), bladder, lung (small cell), breast cancer, and retina (retinoblastoma). Mutations in the Wilms Tumour-1 gene (WT-1) are associated with nephroblastoma and neurofibromatosis. Mutations in MADR2 are linked with colorectal cancer (6% of sporadic colorectal cancers).

The tumour suppressor gene that has been the subject of the most research is p53. p53 encodes a protein which functions as a transcription factor and is a key regulator of the cell division cycle. The p53 gene is mutated in at least 50% of human tumours. Genes regulated by the transcriptional activity of p53 contain a p53 recognition sequence in their 5′ regions. In response to a variety of cellular stresses, p53 is post-translationally modified and protein levels increase dramatically. This activation results in the activation of other genes, such as mdm2 (Momand et al., Cell 69:1237-45, 1992), Bax (Miyashita and Reed, Cell 80:293-9, 1995) and PIG-3 (Polyak et al., Nature 389, 300-5, 1997). Activation of p53 protein results in either arrest of the cell at G1 or commitment to death through apoptosis. Bax and PIG-3 are involved in the induction of apoptosis function of p53. Apoptosis, or programmed cell death, is a natural process that removes damaged cells, and is important in the removal of pre-cancerous cells, cell/tissue development and homeostasis. However p53 can induce apoptosis by both transcriptional dependent and independent mechanisms (Volgelstein et al., Nature 408:307-10, 2000; Vousden and Lu, Nat. Rev. Cancer 2:594-604, 2002). The ability of p53 to induce apoptosis is an important tumour suppression function. p53 induced-apoptosis can be blocked by the oncogene bcl-2. However, bcl-2 does not inhibit the transactivation function of p53.

p53 is a member of a family of three proteins; p53, p63 and p73. Both p63 and p73 share over 60% amino acid identity within the DNA binding region of p53 (Jost et al., Nature 389:191-4, 1997; Kaghad et al., Cell 90:809-19, 1997; Yang et al., Molecular Cell 2:305-16, 1998). The DNA binding specificity among p53 family members are similar, but not identical. As a result, a large number of p53 target genes are transactivated by p63 and p73. Hence, p63 and p73 share some p53 functions such as cell cycle arrest and apoptosis.

However, there are structural and functional differences between p53 and its family members p63 and p73. For example, mutations in p63 and p73 are rare in human cancer. Expression of p63 and p73 is more important for mouse development than p53, and loss of p73 or p63 did not predispose mice to cancer (Yang et al., 2002. Trends Genet. 18:90-5, 2002). Cellular regulators of p53, such as mdm2, do not have the same effects on p63 and p73. While the binding of mdm2 to p53 inhibits the transactivation function of p53 and targets it for degradation (Haupt et al., Nature 387:296-9, 1997; Kubbutat et al., Nature 387:299-303, 1997), it fails to target p63 and p73 for degradation (Balint et al., Oncogene 18:3923-9, 1999; Dobbelstein et al., Oncogene 18:2101-6, 1999). In contrast, the binding of mdm2 to p63 even stimulated the transactivation function of p63 by stabilizing the protein (Calabro et al., J. Biol. Chem. 277:2674-81, 2002). Similarly, the CCAAT-binding transcription factor CTF2 binds to the DNA binding region of p53 and p73 but leads to different biological consequences. The binding of CTF2 to p53 enhances the DNA binding activity of p53 but the interaction of CTF2 to p73 inhibits the DNA binding activity of p73 (Uramoto et al., Biochem J. 371:301-10, 2003). Moreover, unlike p53, p63 and p73 do not interact with viral proteins such as the large T antigen of SV40 through their DNA binding domain (Dobbelstein and Roth, J. Gen. Virol. 79 (Pt 12):3079-83, 1998; Dobbelstein et al., Oncogene 18:2101-6, 1999; Marin et al., Mol. Cell. Biol. 18:6316-24, 1998).

These results indicate that an activator or an inhibitor of p53 does not necessarily have similar physiological implications on its family members p63 and p73. This could explain why no universal activator or inhibitor of the p53 family members has yet been identified. However, it would be beneficial if a universal activator of p53 family members was identified, as such agents could be used to induce apoptosis.

### SUMMARY

The inventor has demonstrated that the apoptotic function of p53 is significantly enhanced by two novel apoptosis stimulating proteins (ASPP's) ASPP1 and ASPP2. In addition to being an activator of p53, ASPP1 and ASPP2 also induce apoptosis independent of p53, and enhance the apoptotic function of the p53 family members, p63 and p73. ASPP1 and ASPP2 are shown herein to bind to p53, p63, and p73 in vitro and in vivo. By binding to the most conserved and homologous region of the p53 family members, the DNA binding domain, ASPP1 and ASPP2 specifically stimulate the transactivation function of p53 family members on the promoters of Bax but not mdm2. Consequently, ASPP1 and ASPP2 increase the apoptotic function of p53 family members, including p53, p63 and p73. The removal of endogenous p63 or p73 with RNAi of p63 and p73 demonstrated that the p53 independent apoptotic function of ASPP1 and ASPP2 is mediated mainly by p63 and p73. Therefore, ASPP1 and ASPP2 are the first two identified common activators of all p53 family members.

Methods are provided for using ASPP1 and ASPP2 (as well as variants, fragments and fusions thereof that retain the ability to enhance the apoptotic function of p53, p63 and p73) to enhance apoptosis, for example to suppress tumour growth, such as in tumors that express mutant p53 or do not express p53. In particular examples, the method includes screening a subject to detect the presence of p53 (mutant or wild-type), p63, or p73-expressing tumor. Subjects having such tumors would benefit from the disclosed therapies. Subjects identified as having a p53 (mutant or wild-type), p63, or p73-expressing tumor would then be administered the therapies disclosed herein, such as administration of an ASPP1 or ASPP2 protein (or nucleic acid encoding such a protein), including variants, fragments and fusions thereof that retain the ability to enhance the apoptotic function of p53, p63 and p73. Such therapies can be administered alone or in combination with other agents, such as other anti-tumor agents. The additional agents can be administered before, during, or after administration of an ASPP1 or ASPP2 protein (or nucleic acid encoding such a protein).

Methods of screening for agents that modify the activity of p63 or p73 are also disclosed. For example, agents that increase the activity of p63 or p73 can be used to increase apoptosis (for example by at least 10%, at least 20%, or even at least 50%, as compared to an amount of apoptosis in the absence of the agent). In other examples, agents that decrease p63 or p73 activity can be used to decrease apoptosis (for example by at least 10%, at least 20%, or even at least 50%, as compared to an amount of apoptosis in the absence of the agent). In one example, the effect of the test agent on the binding between ASPP1 or ASPP2 and a p53 family member is detected. In another example, the effect of the test agent on apoptosis in the presence of ASPP1 or ASPP2 and a p53 family member is determined.

The ASPP2 sequence was identified as follows. Antibodies to 53BP2 were generated. Endogenous bBP2/53BP2 was found to encode a protein larger than the 1005 amino acids predicted by Naumovski and Cleary (Mol. Cell. Biol. 16:3884-92, 1996). This protein, which consists of 1128 amino acids, was named ASPP2 (SEQ ID NO: 4). For the sake of clarity the following nomenclature will be used. The 528 amino acid polypeptide will be referred to as 53BP2 or ASPP2/53BP2 (600-1128); the 1005 amino acid polypeptide will be referred to as bBP2/53BP or ASPP2/Bbp2 (123-1128); and the 1128 amino acid polypeptide will be referred to as ASPP2/53BP, or simply ASPP2 (1-1128). The numbers in parenthesis indicate the equivalent amino acids of ASPP2. A cDNA sequence of ASPP2 is shown in SEQ ID NO: 3.

It is shown herein that the C-terminal half of bBP2/53BP does not have a significant effect on the activity of p53. However, ASPP2/53BP enhanced the transactivation function of p53 on the promoters derived from pro-apoptosis related genes such as Bax and PIG-3.

Using the cDNA sequence of ASPP2, a BLAST search identified GenBank Accession No: KIAA0771 having significant homology to the nucleic acid sequence encoding bBP2/BP53. This member of the family is referred to herein as Apoptosis Stimulating Protein 1 (ASPP1). Using a PCR-RACE kit as described by the manufacturer, 100 bp of ASPP1 cDNA 5′-upstream to KIAA0771 was cloned and used in a BLAST search, which identified another EST clone (EMBO entry AI625004). We obtained the EST clones AI625004 and KIAA0771 were subcloned together to generate the full length clone of ASPP1 cDNA as shown in SEQ ID NO: 1.

The sequence homologies between ASPP1 and ASPP2, at the level of protein sequence, is shown in FIG. 1. The highest homology between ASPP1 and ASPP2 is found in the N- and C-terminal regions of the protein. ASPP1 is encoded by a gene located on chromosome 14. Most of the exons and introns are within the genomic clone under EMBO entry AL049840. The promoter region and the 5′ end exons and introns are located within the genomic clone EMBO entry CNS01DTD.

Disclosed herein is a novel regulator of ASPP2, termed iASPP, which inhibits the p53-stimulatory effect of ASPP2. In tumours expressing ASPP1 and ASPP2, expression of iASPP is up-regulated compared to the matched normal controls. Therefore, the tumour suppression function of p53 can be positively and negatively regulated by ASP and iASPP in vivo.

Binding to the DNA binding domain of p53, ASPP1 and ASPP2 specifically stimulates the transactivation function of p53 on promoters of pro-apoptotic genes such as Bax and PIG3 but not on promoters of p21waf1 or mdm2. Since the DNA binding domain of p53 is the most homologous region among all p53 family members, we investigated whether ASPP1 and ASPP2 can also interact with the rest of the p53 family members, p63 and p73. The effects of ASPP1 and ASPP2 on the transactivation and apoptotic function of p63 and p73 were also studied.

The foregoing and other objects, features, and advantages of the disclosure will become more apparent from the following detailed description of a several embodiments that proceeds with reference to the accompanying figures.

### BRIEF DESCRIPTION OF THE FIGURES

FIG. 1 shows sequence homologies between ASPP1, ASPP2 and iASPP.

FIGS. 2A and 2B are bar graphs showing the stimulation of various p53 specific promoters in the presence of combinations of p53, (A) ASPP1 and (B) ASPP2.

FIGS. 2C and 2D are bar graphs showing the stimulation of p53 transactivation by (C) ASPP1 and (D) ASPP2.

FIG. 3 is a bar graph showing the stimulation of p53 transactivation by various lengths of ASPP2 peptide.

FIGS. 4A and 4B are bar graphs showing the synergistic effect of ASPP1 and ASPP2 on the apoptotic function of p53.

FIG. 4C is a bar graph showing the dominant negative effect of the C-terminal half of ASPP2 on the apoptotic function of p53.

FIG. 4D is a bar graph showing the synergistic effect of ASPP2 on the apoptotic function of p53, p73 and p63.

FIG. 5A is a bar graph showing the induction of p53 induced apoptosis by ASPP1 and ASPP2 and the inhibition of p53-induced apoptosis by iASPP.

FIG. 5B is a bar graph showing the activation of p53 responsive promoter, Bax by ASPP1 and ASPP2 and inhibition of transactivation by iASPP.

FIG. 6A is a bar graph showing the percentage of cells with sub-G1 DNA content (apoptotic cells) expressing p53 or p53 mutants in the presence or absence of ASPP1 or ASPP2.

FIG. 6B is a bar graph showing the transcriptional activity of p53 or p53 mutants and the influence of ASPP1 or ASPP2.

FIG. 7A is a bar graph showing that the apoptotic function of p53 is highly regulated by ASP family members in vivo. The bar graphs represent the percentage of transfected cells with sub-G1 DNA content, characteristic of apoptosis.

FIG. 7B is a bar graph showing the dominant negative function of 53BP2 and iASPP in inhibiting apoptosis induced by endogenous p53 in response to DNA damage with cisplatin.

FIG. 7C is a bar graph showing that co-expression of antisense ASPP1 or ASPP2 did not influence apoptosis mediated by Bax.

FIG. 7D is a bar graph showing endogenous ASPP1 and ASPP2 are involved in regulating the apoptotic function of p53 in response to DNA damage.

FIG. 7E is a bar graph showing that antisense iASPP enhanced the apoptotic function of ASPP1 and ASPP2.

FIG. 8A illustrates a model describing the interaction of ASP family members with p65, IkB and p53.

FIGS. 8B and 8C are bar graphs showing the ability of IkB affect the transactivation function of p53 on Bax and mdm2 promoters in the presence and absence of ASPP2.

FIG. 9A is a bar graph showing the ability of Bcl-2 to inhibit the stimulating effect of ASPP1 and ASPP2 on p53H175-L-induced apoptosis.

FIG. 9B is a bar graph showing the inability of Bcl-XL to inhibit the stimulating effect of ASPP1 and ASPP2 on p53 H175-L -induced apoptosis

FIG. 9C is a bar graph showing the ability of Bcl-2 to inhibit p53-induced apoptosis by ASPP1 and ASPP2.

FIG. 10A is a bar graph showing the enhancing effect of iASPP on the transforming function of E7.

FIG. 10B is a bar graph showing the enhancing effect of iASPP on cell resistance to cisplatin.

FIGS. 11A-D are bar graphs and digital images of Western blots showing that ASPP1 and ASPP2 can induce apoptosis independent of p53 in Saos-2 (A, B) and H1299 (C, D) cells.

FIG. 12A is a sequence comparison of the DNA binding domains of p53, p63 and p73, demonstrating that the majority of the residues involved in ASPP binding are conserved. p53, p63 and p73 sequences were obtained from Genbank and aligned using CLUSTAL W. The ASP contact residues are indicated with arrows.

FIGS. 12B and 12C are digital images of western blots showing that ASPP1 and ASPP2 interact with p53 and its family members in vitro.

FIGS. 13A-D are digital images of western blots showing that ASPP1 and ASPP2 can interact with p63γ and p73α in vivo.

FIGS. 13E-F are digital images of western blots showing that when large amounts of cell lysate were used, the interaction between endogenous ASPP2 and p63γ or p73α was detected.

FIGS. 14A-C are bar graphs and digital images of Western blots showing that ASPP1 and ASPP2 can specifically stimulate the transactivation function of p53 family members on promoters of pro-apoptotic genes such as Bax, but not mdm2. The bar graphs show the effects of ASPP1 and ASPP2 on the transactivation function of p53, p63γ or p73α on the Bax-luc promoter as indicated (A and B). The fold increase in p53, p63γ or p73α transactivation activity by either ASPP1 or ASPP2 on two p53 reporters, Bax and mdm2 luciferase (C).

FIG. 15 is a bar graph showing that ASPP1 and ASPP2 specifically stimulate the apoptotic function of p53, p63γ and p73α. The bar graph represents the percentage of apoptotic cells 36 hours after transfection and was derived from two independent experiments.

FIGS. 16A-16C are bar graphs and digital images of Western blots showing the ability of p63 and p73 RNAi to reduce apoptosis induced by p63 and p73. The ability of p63 and p73 RNAi to inhibit the expression of p63 and p73 is shown in the lower panel of FIG. 6A. The bar graph represents the percentage of apoptotic cells 36 hours after transfection and was derived from two independent experiments.

FIG. 17 is a sequence comparison showing that three out of eight ASPP2 binding residues are not identical in p63 and p73 even though these are conserved among p53 from different species. p63 p73 and p53 sequences from various species were obtained from Genbank and aligned using CLUSTAL W. Conserved residues between the two family members are indicated by shaded residues. The conserved ASPP contact residues are indicated with arrows.

### SEQUENCE LISTING

The nucleotide and protein sequences described herein are shown using standard letter abbreviations for nucleotide bases, and three letter code for amino acids. Only one strand of each nucleic acid sequence is shown, but the complementary strand is understood as included by any reference to the displayed strand.

SEQ ID NO: 1 is an ASPP1 cDNA sequence.

SEQ ID NO: 2 is an ASPP1 protein sequence encoded by SEQ ID NO: 1.

SEQ ID NO: 3 is an ASPP2 cDNA sequence.

SEQ ID NO: 4 is an ASPP2 protein sequence encoded by SEQ ID NO: 3.

SEQ ID NO: 5 is an iASPP cDNA sequence.

SEQ ID NO: 6 is an iASPP protein sequence encoded by SEQ ID NO: 5.

SEQ ID NO: 7 is a sense p63 oligonucleotide.

SEQ ID NO: 8 is an antisense p63 oligonucleotide.

SEQ ID NO: 9 is a sense p73 oligonucleotide.

SEQ ID NO: 10 is an antisense p73 oligonucleotide.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid” includes single or plural nucleic acids and is considered equivalent to the phrase “comprising at least one nucleic acid.” The term “or” refers to a single element of stated alternative elements or a combination of two or more elements, unless the context clearly indicates otherwise. For example, the phrase “a first nucleic acid or a second nucleic acid” refers to the first nucleic acid, the second nucleic acid, or a combination of both the first and second nucleic acids. As used herein, “comprises” means “includes.” Thus, “comprising a promoter and an open reading frame,” means “including a promoter and an open reading frame,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

ASPP: Apoptosis Stimulating Protein

Agent: Any substance, including, but not limited to, an antibody, chemical compound, molecule, peptidomimetic, or protein.

Antisense, Sense, and Antigene. Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a particular dsDNA target. These molecules can be used to interfere with gene expression.

Double-stranded DNA (dsDNA) has two strands, a 5′ to 3′ strand, referred to as the plus (+) strand, and a 3′ to 5′ strand (the reverse complement), referred to as the minus (−) strand. Because RNA polymerase adds nucleic acids in a 5′ to 3′ direction, the minus strand of the DNA serves as the template for the RNA during transcription. Thus, the RNA formed will have a sequence complementary to the minus strand and virtually identical to the plus strand, except that U is substituted for T in RNA molecules.

Apoptosis: The process of programmed cell death, the deliberate suicide of a cell. Apoptosis can be characterized by the loss of cell junctions and microvilli, condensation of the cytoplasm, margination of the nuclear chromatin, fragmentation of the nucleus, followed by formation of apoptotic bodies. In some examples, cancerous cell are unable to undergo apoptosis.

ASPP1: Includes any ASPP1 nucleic acid molecule or protein from any organism that has ASPP1 activity, such as the ability to bind to p53, p63, and p73, the ability to increase the promoter activity of Bax, the ability to increase the apoptotic function of p53, p63, and p73, or combinations thereof. In particular examples provided herein, the ASPP1 is a mammalian ASPP1, such as a mouse or human ASPP1.

An example of a native ASPP1 nucleic acid sequence includes, but is not limited to: SEQ ID NO: 1, such as nucleotides 159-3431 of SEQ ID NO: 1. An example of a native ASPP1 protein sequence includes, but is not limited to: SEQ ID NO: 2. In one example, an ASPP1 sequence includes a full-length wild-type (or native) sequence, as well as ASPP1 allelic variants, variants, fragments, homologs or fusion sequences that retain the ability to bind to p53, p63, and p73, the ability to increase the promoter activity of Bax, the ability to increase the apoptotic function of p53, p63, and p73, or combinations thereof. In certain examples, ASPP1 has at least 80% sequence identity, for example at least 85%, at least 90%, at least 95%, at least 98%, or at least 99% sequence identity to a native ASPP1. In particular examples, an ASPP1 protein includes at least 9 amino acids, such as at least 10 amino acids, at least 20 amino acids, at least 50 amino acids, at least 100 amino acids, or even at least 1000 amino acids, for example 9-1000 amino acids.

ASPP2: Includes any ASPP2 nucleic acid molecule or protein from any organism that has ASPP2 activity, such as the ability to bind to p53, p63, and p73, the ability to increase the promoter activity of Bax, the ability to increase the apoptotic function of p53, p63, and p73, or combinations thereof. In particular examples provided herein, the ASPP2 is a mammalian ASPP2, such as a mouse or human ASPP1.

An example of a native ASPP2 nucleic acid sequence includes, but is not limited to: SEQ ID NO: 3, such as nucleotides 256-3642 of SEQ ID NO. 3. An example of a native ASPP2 peptide includes, but is not limited to: SEQ ID NO: 4. In one example, an ASPP2 sequence includes a full-length wild-type (or native) sequence, as well as ASPP2 allelic variants, variants, fragments, homologs or fusion sequences that retain the ability to bind to p53, p63, and p73, the ability to increase the promoter activity of Bax, the ability to increase the apoptotic function of p53, p63, and p73, or combinations thereof. In certain examples, ASPP2 has at least 80% sequence identity, for example at least 85%, at least 90%, at least 95%, at least 98%, or at least 99% sequence identity to a native ASPP2. In particular examples, an ASPP2 protein includes at least 9 amino acids, such as at least 10 amino acids, at least 20 amino acids, at least 50 amino acids, at least 100 amino acids, or even at least 1000 amino acids, for example 9-1000 amino acids.

ASPP-activity: The ability of an ASPP agent, to bind to p53, p63, and p73, the ability to increase the promoter activity of Bax, the ability to increase the apoptotic function of p53, p63, and p73, or combinations thereof. ASPP agents include, but are not limited to, ASPP1 and ASPP2 proteins (including variants, fusions, fragments and mimetics thereof), nucleic acid molecules (including DNA and RNA molecules), specific binding agents, mimetics thereof, and agonists.

In particular examples, ASPP activity occurs when ASPP1 or ASPP2 proteins, nucleic acid molecules, specific binding agents, agonists, or mimetics thereof, bind to p53, p63, or p73, and can thereby increase the apoptotic function of p53, p63, or p73, for example by at least 10%, at least 50%, at least 100%, or even at least 200%, as compared to an amount of apoptosis in the absence of such agents. In another example ASPP activity occurs when ASPP1 or ASPP2 proteins, nucleic acid molecules, specific binding agents, agonists, or mimetics thereof increase the promoter activity of Bax, for example by at least 10%, at least 50%, at least 100%, at least 200%, or even at least 1000%, as compared to an amount of promoter activity in the absence of such agents.

Assays are described herein that can be used to determine if an agent has ASPP activity or reduces that activity, for example as shown in EXAMPLES 3-6 and 14-18.

Cancer: Malignant neoplasm that has undergone characteristic anaplasia with loss of differentiation, increase rate of growth, invasion of surrounding tissue, and is capable of metastasis.

cDNA (complementary DNA): A piece of DNA lacking internal, non-coding segments (introns) and regulatory sequences that determine transcription. cDNA can be synthesized in the laboratory by reverse transcription from messenger RNA extracted from cells.

Chemical synthesis: An artificial means by which a protein can be generated.

Chemotherapeutic agent: In cancer treatment, chemotherapy refers to the administration of one or a combination of compounds to kill or slow the reproduction of rapidly multiplying cells. Exemplary chemotherapeutic agents include, but are not limited to: cisplatin; carboplatin; oxaliplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; an enediyne such as chalicheamicin or esperamicin; chlorambucil; ARA-C; vindesine; bleomycin; etoposide, and combinations thereof.

Chemotherapy-resistant disease: A disorder that is not responsive to solely administration of a chemotherapeutic agent.

Conservative substitution: A substitution of an amino acid residue for another amino acid residue having similar biochemical properties. Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions. For example, a conservative substitution in an ASPP1 or ASPP2 peptide does not substantially affect the ability of the peptide to increase the apoptotic function of p53, p63, or p73. In addition, a conservative substitution in an iASPP peptide does not substantially affect the ability of the peptide to decrease apoptosis induced by p53 in the presence of ASPP1 or ASPP2.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

Examples of amino acids which can be substituted for an original amino acid in a protein and which are regarded as conservative substitutions include, but are not limited to: Ser for Ala; Lys for Arg; Gln or His for Asn; Glu for Asp; Ser for Cys; Asn for Gln; Asp for Glu; Pro for Gly; Asn or Gln for His; Leu or Val for Ile; Ile or Val for Leu; Arg or Gln for Lys; Leu or Ile for Met; Met, Leu or Tyr for Phe; Thr for Ser; Ser for Thr; Tyr for Trp; Trp or Phe for Tyr; and Ile or Leu for Val.

Further information about conservative substitutions can be found in, among other locations in, Ben-Bassat et al., (J. Bacteriol. 169:751-7, 1987), O'Regan et al., (Gene 77:237-51, 1989), Sahin-Toth et al., (Protein Sci. 3:240-7, 1994), Hochuli et al., (Bio/Technology 6:1321-5, 1988) and in standard textbooks of genetics and molecular biology.

Decrease: To reduce the quality, amount, or strength of something. In one example, a therapy decreases growth or metastasis of a tumor if growth or metastasis of the tumor is reduced as compared to growth in the absence of the therapy. In a particular example, increased levels of ASPP1 or ASPP2 decrease growth or metastasis of a tumor in a subject. Such reduction can be measured, for example, by determining the volume of the tumor, by determining if metastases are present, determining a symptom associated with the presence of the tumor, or combinations thereof.

Degenerate variant: A polynucleotide sequence encoding a polypeptide that includes a sequence that is degenerate as a result of the genetic code. There are 20 natural amino acids, most of which are specified by more than one codon. For example, serine residues are encoded by the codons TCA, AGT, TCC, TCG, TCT and AGC. Each of the six codons is equivalent for the purposes of encoding a serine residue. Therefore, all degenerate nucleotide sequences are included as long as the amino acid sequence of the polypeptide encoded by the nucleotide sequence is unchanged.

Deletion: The removal of one or more nucleotides from a nucleic acid sequence (or one or more amino acids from a protein sequence), the regions on either side of the removed sequence being joined together.

DNA (deoxyribonucleic acid): A long chain polymer which includes the genetic material of most living organisms (some viruses have genes including ribonucleic acid, RNA). The repeating units in DNA polymers are four different nucleotides, each of which includes one of the four bases, adenine, guanine, cytosine and thymine bound to a deoxyribose sugar to which a phosphate group is attached. Triplets of nucleotides, referred to as codons, in DNA molecules code for amino acid in a polypeptide. The term codon is also used for the corresponding (and complementary) sequences of three nucleotides in the mRNA into which the DNA sequence is transcribed.

Dominant negative peptide: An inactive variant of a protein, which can displace an active protein from its interaction with the cellular machinery or competes with the active protein, thereby reducing the effect of the active protein. For example, a dominant negative receptor that binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to another transcription factor or to a promoter site in the control region of a gene but does not increase gene transcription can reduce the effect of a normal transcription factor by occupying promoter binding sites without increasing transcription.

The result of expressing a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, the sequence of native ASPP1, ASPP2 or iASPP peptides can be mutated by site-specific mutagenesis, scanning mutagenesis, partial gene deletion or truncation, and the like (for example see U.S. Pat. No. 5,580,723 and Sambrook et al., Molecular Cloning: A Laboratory Manual, Second Edition, Cold Spring Harbor Laboratory Press, 1989). The population of mutagenized peptides can be tested for diminution in a selected activity (such as p53, p63 or p73 binding, modulation of apoptosis), or for retention of such an activity.

Enhance: To improve the quality, amount, or strength of something. In one example, a therapy enhances the promoter activity of Bax, enhances the ability to increase the apoptotic function of p53, p63, and p73, or combinations thereof. In a particular example, ASPP1 or ASPP2 enhances the promoter activity of Bax in a subject having a tumor. In a particular example, ASPP1 or ASPP2 enhances the apoptotic function of p53, p63, or p73 in a subject having a tumor, such as a tumor that expresses p63 or p73. In a particular example, ASPP1 or ASPP2 enhances the apoptotic function of p63 or p73 in a subject having a tumor that does not expresses p53 or expresses a mutant p53. Such enhancement can be measured using any bioassay known in the art, for example, an apoptosis assay as described in Example 6 or a transactivation assay described in Example 4.

In some examples, a therapy enhances the apoptotic function of p53, p63, or p73, or enhances the promoter activity of Bax, if such therapy decreases or halts the progression or size of a tumor, as compared to an amount in the absence of the therapy.

Functional deletion or disruption: A deletion or mutation of a nucleic acid molecule or amino acid sequence that substantially decreases the biological activity of the nucleic acid or amino acid sequence. In one example, the function of a gene or gene product is reduced or eliminated by a deletion, insertion, or substitution. For example, functional deletion of ASPP1 or ASPP2 reduces or can even eliminate detectable ASPP1 or ASPP2 activity, such as the ability of ASPP1 or ASPP2 to increase the apoptotic function of p53, p63, and p73. For example, functional deletion of iASPP reduces or can even eliminate detectable iASPP activity, such as the ability of iASPP to decrease apoptosis induced by p53 in the presence of ASPP1 or ASPP2.

Functionally equivalent: A protein or nucleic acid sequence that includes one or more sequence alterations, wherein the sequence retains a specified function of a native sequence. For example, a functionally equivalent ASPP1 or ASPP2 protein retains the ability to increase the apoptotic function of p53, p63, and p73, increase the promoter activity of Bax, or combinations thereof, as compared to an amount of apoptosis or transactivation in the absence of detectable ASPP1 or ASPP2. For example, a functionally equivalent iASPP protein retains the ability to decrease apoptosis induced by p53 in the presence of ASPP1 or ASPP2 as compared to an amount of apoptosis in the absence of detectable iASPP.

Examples of sequence alterations include, but are not limited to, substitutions, deletions, mutations, frameshifts, and insertions. In one example, a peptide binds an antibody, and a functional equivalent is a peptide that binds the same antibody. Thus a functional equivalent includes peptides which have the same binding specificity as a polypeptide, and which may be used as a reagent in place of the polypeptide (such as in a therapeutic composition). In one example a functional equivalent includes a polypeptide wherein the binding sequence is discontinuous, wherein the antibody binds a linear epitope. Thus, if the peptide sequence is MMPMILTVFL (amino acids 1-10 of SEQ ID NO: 2, the N-terminal 10 amino acids of a human ASPP1 protein) a functional equivalent includes discontinuous epitopes, which may can appear as follows (**=any number of intervening amino acids): NH2-**-M**M**P**M**I**L**T**V**F**L-COOH. This polypeptide is functionally equivalent to SEQ ID NO: 2 if the three dimensional structure of the polypeptide is such that it can bind a monoclonal antibody that binds SEQ ID NO: 2.

Hybridization: To form base pairs between complementary regions of two strands of DNA, RNA, or between DNA and RNA, thereby forming a duplex molecule. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (detects sequences that share 90% identity)

| | |
|---|---|
| Hybridization: | 5 × SSC at 65° C. for 16 hours |
| Wash twice: | 2 × SSC at room temperature (RT) for 15 minutes each |
| Wash twice: | 0.5 × SSC at 65° C. for 20 minutes each |

High Stringency (detects sequences that share 80% identity or greater)

| | |
|---|---|
| Hybridization: | 5×-6× SSC at 65° C.-70° C. for 16-20 hours |
| Wash twice: | 2 × SSC at RT for 5-20 minutes each |
| Wash twice: | 1 × SSC at 55° C.-70° C. for 30 minutes each |

Low Stringency (detects sequences that share greater than 50% identity)

| | |
|---|---|
| Hybridization: | 6 × SSC at RT to 55° C. for 16-20 hours |
| Wash at least | 2×-3× SSC at RT to 55° C. for 20-30 minutes each. |
| twice: | |

Insertion: The addition of one or more nucleotides to a nucleic acid sequence, or the addition of one or more amino acids to a protein sequence.

iASPP: Includes any iASPP nucleic acid molecule or protein from any organism that has iASPP activity, such as the ability to decrease the apoptotic function of p53 in the presence of ASPP1 or ASPP2. In particular examples provided herein, iASPP has activity against a mammalian ASP, such as a mouse or human ASP.

Examples of native iASPP nucleic acid sequences include, but are not limited to: SEQ ID NO: 5, and the sequence provided in GenBank Accession No. NM_{—}073554. Examples of native iASPP protein sequences include, but are not limited to: SEQ ID NO: 6, and the sequence provided in GenBank Accession No. NP_{—}505955. In one example, an iASPP sequence includes a full-length wild-type (or native) sequence, as well as iASPP allelic variants, variants, fragments, homologs or fusion sequences that retain the ability to decrease the apoptotic function of p53 in the presence of ASPP1 or ASPP2. In certain examples, iASPP has at least 80% sequence identity, for example at least 85%, at least 90%, at least 95%, at least 98%, or at least 99% sequence identity to a native iASPP. In particular examples, an iASPP protein includes at least 9 amino acids, such as at least 10 amino acids, at least 20 amino acids, at least 50 amino acids, at least 100 amino acids, or even at least 1000 amino acids, for example 9-1000 amino acids.

iASPP activity: The ability of an iASPP agent to decrease the apoptotic function of p53 in the presence of ASPP1 or ASPP2. iASPP agents include, but are not limited to, iASPP proteins (including variants, fusions, fragments and mimetics thereof), nucleic acid molecules (including DNA and RNA molecules), specific binding agents, mimetics thereof, and agonists.

In particular examples, iASPP activity occurs when iASPP proteins, nucleic acid molecules, specific binding agents, agonists, or mimetics thereof, decrease the apoptotic function of p53 in the presence of ASPP1 or ASPP2, for example by at least 10%, at least 50%, at least 100%, or even at least 200%, as compared to an amount of apoptosis in the absence of such agents. Assays are described herein that can be used to determine if an agent has iASPP activity or reduces that activity, for example as shown in Example 7.

Isolated: An “isolated” biological component (such as a nucleic acid molecule, protein, or organelle) has been substantially separated or purified away from other biological components in the cell of the organism in which the component naturally occurs, such as other chromosomal and extra-chromosomal DNA and RNA, proteins and organelles. Nucleic acids and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Mammal: This term includes both human and non-human mammals.

Mediated condition: A disease or disorder that is associated with defects in one or more genes, such as expression levels of one or more genes.

For example, a p53 mediated condition is a disease associated with defects in p53 biological activity, such as tumor development. Because mutations in p53 sequences are associated with many human cancers, cancer is a p53 mediated condition.

For example, a p63 mediated condition is a disease associated with defects in p63 biological activity, such as defects in ectodermal development. Because p63-deficient mice have a defective apical ectodermal ridge, truncated limbs, no teeth, no hair follicles, no mammary, lachrymal, or salivary glands, such disorders are p63 mediated conditions. A particular p63 mediated condition is ectrodactyly, ectodermal dyslasia and facial clefts (EEC syndrome) which results from p63 mutations. In a particular example, non-small cell lung carcinoma is a p63 mediated condition.

For example, a p73 mediated condition is a disease associated with defects in p73 biological activity, such as defects in development. Because p73-deficient mice have congenital hydrocephalus, hippocampal dysgenesis, defects of pheromone detection, and pan-mucositis, such disorders are p73 mediated conditions. A particular p73 mediated condition is ectrodactyly, ectodermal dyslasia and facial clefts (EEC syndrome) which results from p73 mutations. In a particular example, neuroblastoma, lung cancer or ovarian cancer is a p73 mediated condition.

Mimetic: An ASPP1 or ASPP2 mimetic includes variants, fragments of fusions of ASPP1 or ASPP2 peptides, as well as organic compounds and modified ASPP1 or ASPP2 peptides, which retain ASPP1 or ASPP2 activity, respectively. In one example, a mimetic mimics the increase in the promoter activity of Bax, the increase the apoptotic function of p53, p63, and p73, or combinations thereof, generated by ASPP1 or ASPP2.

An iASPP mimetic includes variants, fragments of fusions of iASPP peptides, as well as organic compounds and modified iASPP peptides, which retain iASPP activity, respectively. In one example, a mimetic mimics the decrease of p53 apoptotic function in the presence of ASPP1 or ASPP2, generated by iASPP.

Modulate: To increase or decrease.

Nucleic acid molecules: A deoxyribonucleotide or ribonucleotide polymer including, without limitation, cDNA, mRNA, genomic DNA, and synthetic (such as chemically synthesized) DNA. Nucleic acid molecules can be double-stranded or single-stranded. Where single-stranded, the nucleic acid molecule can be the sense strand or the antisense strand. In addition, nucleic acid molecules can be circular or linear.

The disclosure includes isolated nucleic acid molecules that include specified lengths of an ASPP1, ASPP2, or iASPP nucleotide sequence. For example, such molecules can include at least 10, 15, 20, 25, 30, 35, 40, 45, 50, 100, 200, 300, 500, 1000, 2000, 3000, 3500, or 4000 consecutive nucleotides of these sequences or more, and can be obtained from any region of an ASPP1, ASPP2, or iASPP nucleic acid molecule.

Nucleotide: Includes, but is not limited to, a monomer that includes a base linked to a sugar, such as a pyrimidine, purine or synthetic analogs thereof, or a base linked to an amino acid, as in a peptide nucleic acid (PNA). Includes analogues of natural nucleotides that hybridize to nucleic acid molecules in a manner similar to naturally occurring nucleotides. A nucleotide is one monomer in a polynucleotide. A nucleotide sequence refers to the sequence of bases in a polynucleotide.

Oligonucleotide: An oligonucleotide is a plurality ofjoined nucleotides joined by native phosphodiester bonds, between about 6 and about 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 bases, for example at least 8, 10, 15, 20, 25, 30, 35, 40, 45, 50, 100 or even 200 nucleotides long, or from about 6 to about 50 nucleotides, for example about 10-25 nucleotides, such as 12, 15 or 20 nucleotides.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

p53: Includes any p53 nucleic acid molecule or protein from any organism that has p53 activity, such as the ability to decrease or suppress tumor growth or development, the ability to regulate the cell cycle, the ability to induce apoptosis, the ability to function as a transcription factor, or combinations thereof. In particular examples provided herein, p53 is a mammalian p53, such as a mouse or human p53.

Examples of native p53 nucleic acid sequences include, but are not limited to: GenBank Accession No. M13872 (mouse), GenBank Accession No. AH002222 (rat), and GenBank Accession No. M14695 (human). Examples of native p53 protein sequences include, but are not limited to: GenBank Accession No. AAA39883 (mouse), GenBank Accession No. AAA41788 (rat), and GenBank Accession No. AAA61212 (human). In one example, a p53 sequence includes a full-length wild-type (or native) sequence, as well as p53 allelic variants, variants, fragments, homologs or fusion sequences that retain the ability to induce apoptosis. In certain examples, p53 has at least 80% sequence identity, for example at least 85%, at least 90%, at least 95%, at least 98%, or at least 99% sequence identity to a native p53. In particular examples, a p53 protein includes at least 9 amino acids, such as at least 10 amino acids, at least 20 amino acids, at least 50 amino acids, at least 100 amino acids, at least 200 amino acids, at least 300 amino acids, at least 350 amino acids, for example 9-380 amino acids.

A mutant p53 molecule includes a mutant p53 nucleic acid molecule or protein from any organism that has lost a significant amount of p53 activity. For example, mutant p53 molecules have reduced ability to decrease or suppress tumor growth or development, the ability to regulate the cell cycle, the ability to induce apoptosis, the ability to function as a transcription factor, or combinations thereof. Exemplary mutant p53 sequences are disclosed herein, and also include Yamada et al. (Cancer Res. 51:5800-5, 1991), Mashiyama et al. (Oncogene 6:1313-8, 1991) and Peller et al. (DNA Cell Biol. 14:983-90, 1995) (all herein incorporated by reference).

p63: A p53 homolog that includes any p63 nucleic acid molecule or protein from any organism that has p63 activity, such as the ability to regulate the cell cycle and apoptosis. In some examples, p63 activity includes the ability to regulate ectodermal development, such as development of limbs, hair, teeth, mammary glands, lachrymal glands, or salivary glands. In particular examples provided herein, p63 is a mammalian p63, such as a mouse or human p63.

Examples of native p63 nucleic acid sequences include, but are not limited to: GenBank Accession No. XM_{—}147232 (mouse), GenBank Accession No. NM_{—}019221 (rat), and GenBank Accession No. S78187 (human). Examples of native p63 protein sequences include, but are not limited to: GenBank Accession No. XP_{—}147232 (mouse), GenBank Accession No. NP_{—}062094 (rat), and GenBank Accession No. AAB21139 (human). In one example, a p63 sequence includes a full-length wild-type (or native) sequence, as well as p63 allelic variants, variants, fragments, homologs or fusion sequences that retain the ability to regulate apoptosis. In certain examples, p63 has at least 80% sequence identity, for example at least 85%, at least 90%, at least 95%, at least 98%, or at least 99% sequence identity to a native p63. In particular examples, a p53 protein includes at least 9 amino acids, such as at least 10 amino acids, at least 20 amino acids, at least 50 amino acids, at least 100 amino acids, at least 300 amino acids, at least 500 amino acids, at least 550 amino acids, for example 9-560 amino acids.

p73: A p53 homolog that includes any p73 nucleic acid molecule or protein from any organism that has p73 activity, such as the ability to regulate the cell cycle and apoptosis. In some examples, p73 activity includes the ability to regulate development, such as development of neurological structures. In some examples, p73 does not bind to (and are not inhibited by) viral oncoproteins that bind to p53. In particular examples provided herein, p73 is a mammalian p73, such as a mouse or human p73.

Examples of native p73 nucleic acid sequences include, but are not limited to: GenBank Accession No. AF138873 (mouse) and GenBank Accession Nos. Y11416 and NM_{—}005427 (human). Examples of native p73 protein sequences include, but are not limited to: GenBank Accession No. AAD32213 (mouse), and GenBank Accession Nos. O15350 and CAA72219 (human). In one example, a p73 sequence includes a full-length wild-type (or native) sequence, as well as p73 allelic variants, variants, fragments, homologs or fusion sequences that retain the ability to regulate apoptosis. In certain examples, p73 has at least 80% sequence identity, for example at least 85%, at least 90%, at least 95%, at least 98%, or at least 99% sequence identity to a native p73. In particular examples, a p73 protein includes at least 9 amino acids, such as at least 10 amino acids, at least 20 amino acids, at least 50 amino acids, at least 100 amino acids, at least 300 amino acids, at least 500 amino acids, at least 600 amino acids, for example 9-600 amino acids.

Peptide Modifications: The present disclosure includes ASPP1, ASPP2, and iASPP proteins, as well as synthetic examples of the proteins described herein. In addition, analogues (non-peptide organic molecules), derivatives (chemically functionalized peptide molecules obtained starting with the disclosed peptide sequences) and variants (homologs) of these proteins can be utilized in the methods described herein. For example, ASPP1 or ASPP2 proteins that include modifications, but retain the ability to increase the promoter activity of Bax or the ability to increase the apoptotic function of p53, p63, and p73 can be utilized in the methods described herein. Similarly, iASPP proteins that include modifications, but retain the ability to decrease the apoptotic function of p53 in the presence of ASPP1 or ASPP2 can be utilized in the methods described herein. The peptides disclosed herein include a sequence of amino acids, which can be either L- or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, may be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, may be in the form of a pharmaceutically-acceptable acid addition salt, such as the HCl, HBr, acetic, benzoic, toluene sulfonic, maleic, tartaric and other organic salts, or may be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this invention to select and provide conformational constraints to the structure that result in enhanced stability. For example, a carboxyl-terminal or amino-terminal cysteine residue can be added to the peptide, so that when oxidized the peptide will contain a disulfide bond, generating a cyclic peptide. Other peptide cyclizing methods include the formation of thioethers and carboxyl-and amino-terminal amides and esters.

Peptidomimetic and organomimetic embodiments are also within the scope of the present disclosure, whereby the three-dimensional arrangement of the chemical constituents of such peptido- and organomimetics mimic the three-dimensional arrangement of the peptide backbone and component amino acid side chains, resulting in such peptido-and organomimetics of the proteins of this disclosure having measurable or enhanced ability to bind an antibody. For computer modeling applications, a pharmacophore is an idealized, three-dimensional definition of the structural requirements for biological activity. Peptido-and organomimetics can be designed to fit each pharmacophore with current computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and Principles of Pharmacology Munson (ed.) 1995, Ch. 102, for descriptions of techniques used in CADD. Also included within the scope of the disclosure are mimetics prepared using such techniques.

Pharmaceutical agent or drug: A chemical compound or composition capable of inducing a desired therapeutic or prophylactic effect when properly administered to a subject.

Polynucleotide: A nucleic acid sequence of at least 3 nucleotides. Therefore, a polynucleotide includes molecules which are at least 15, at least 20, at least 30, at least 50, at least 100, at least 200, at least 500, at least 1000, at least 3000, or at least 5000 nucleotides in length, and also nucleotides as long as a full length cDNA. An ASPP1 polynucleotide encodes an ASPP1 peptide, while an ASPP2 polynucleotide encodes an ASPP2 peptide.

Polypeptide: Any chain of amino acids at least six amino acids in length, such as at least 8 amino acids, at least 9 amino acids, at least 20 amino acids, at least 50 amino acids, at least 500 amino acids, at least 1000 amino acids, at least 1100 amino acids, for example about 10-500 or 50-1100 amino acids, regardless of post-translational modification (such as glycosylation or phosphorylation).

Preventing or treating a disease: “Preventing” a disease refers to inhibiting the full development of a disease, for example preventing development or metastasis of a tumor in a person having a tumor that does not express p53 or expresses mutant p53. “Treatment” refers to a therapeutic intervention that ameliorates a sign or symptom of a disease or pathological condition related to the presence of a tumor, such as halting the progression of a tumor, reducing the size of the tumor, or even elimination of the tumor.

Probes and primers: A probe includes an isolated nucleic acid molecule attached to a detectable label or reporter molecule. Exemplary labels include, but are not limited to, radioactive isotopes, ligands, chemiluminescent agents, fluorophores, and enzymes. Methods for labeling and guidance in the choice of labels appropriate for various purposes are discussed, for example in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987).

Primers are short nucleic acid molecules, such as DNA oligonucleotides about at least 15 nucleotides in length. Primers can be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example by PCR or other nucleic-acid amplification methods known in the art.

Methods for preparing and using probes and primers are described, for example, in Sambrook et al. (Molecular Cloning: A Laboratory Manual, Cold Spring Harbor Laboratory Press, 1989), Ausubel et al., 1987, and Innis et al., PCR Protocols, A Guide to Methods and Applications, 1990, Innis et al. (eds.), 21-27, Academic Press, Inc., San Diego, Calif. PCR primer pairs can be derived from a known sequence, for example, by using computer programs intended for that purpose such as Primer (Version 0.5, 1991, Whitehead Institute for Biomedical Research, Cambridge, Mass.).

Promoter: An array of nucleic acid control sequences that directs transcription of a nucleic acid molecule. A promoter includes necessary nucleic acid sequences near the start site of transcription, such as a TATA element. A promoter also optionally includes distal enhancer or repressor elements which can be located as much as several thousand base pairs from the start site of transcription. Both constitutive and inducible promoters are included (Bitter et al., Meth. Enzymol. 153:516-44, 1987).

Specific, non-limiting examples of promoters include promoters derived from the genome of mammalian cells (such as a metallothionein promoter) or from mammalian viruses (such as a retrovirus long terminal repeat; an adenovirus late promoter; a vaccinia virus 7.5K promoter). Promoters produced by recombinant DNA or synthetic techniques can also be used. A nucleotide sequence encoding ASPP1, ASPP2, or iASPP can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Purified: The term purified does not require absolute purity; rather, it is intended as a relative term. Thus, for example, a purified peptide preparation is one in which the peptide or protein is more enriched than the peptide or protein is in its environment within a cell, such that the peptide is substantially separated from cellular components (such as nucleic acid molecules, lipids, carbohydrates, and other polypeptides) that may accompany it. In another example, a purified peptide preparation is one in which the peptide is substantially-free from contaminants, such as those that might be present following chemical synthesis of the peptide.

In one example, an ASPP1, ASPP2, or iASPP peptide is purified when at least 60% by weight of a sample is composed of the peptide, for example when 75%, 95%, or 99% or more of a sample is composed of the peptide. Examples of methods that can be used to purify an antigen, include, but are not limited to the methods disclosed in Sambrook et al. (Molecular Cloning: A Laboratory Manual, Cold Spring Harbor, N.Y., 1989, Ch. 17). Protein purity can be determined by, for example, polyacrylamide gel electrophoresis of a protein sample, followed by visualization of a single polypeptide band upon staining the polyacrylamide gel; high-pressure liquid chromatography; sequencing; or other conventional methods.

Recombinant: A recombinant nucleic acid molecule is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, for example by genetic engineering techniques. Similarly, a recombinant protein is one encoded for by a recombinant nucleic acid molecule.

Sample: A material to be analyzed. Examples include biological samples containing genomic DNA, cDNA, RNA, or protein obtained from the cells of a subject, such as those present in peripheral blood, urine, saliva, tissue biopsy, surgical specimen, fine needle aspriates, amniocentesis samples and autopsy material.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN can be used to compare nucleic acid sequences, while BLASTP can be used to compare amino acid sequences. To compare two nucleic acid sequences, the options can be set as follows: −i is set to a file containing the first nucleic acid sequence to be compared (such as C:\seq1.txt); −j is set to a file containing the second nucleic acid sequence to be compared (such as C:\seq2.txt); −p is set to blastn; −o is set to any desired file name (such as C:\output.txt); −q is set to −1; −r is set to 2; and all other options are left at their default setting. For example, the following command can be used to generate an output file containing a comparison between two sequences: C:\B12seq −i c:\seq1.txt −j c:\seq2.txt −p blastn −o c:\output.txt −q −1 −r2.

To compare two amino acid sequences, the options of B12seq can be set as follows: −i is set to a file containing the first amino acid sequence to be compared (such as C:\seq1.txt); −j is set to a file containing the second amino acid sequence to be compared (such as C:\seq2.txt); −p is set to blastp; −o is set to any desired file name (such as C:\output.txt); and all other options are left at their default setting. For example, the following command can be used to generate an output file containing a comparison between two amino acid sequences: C:\B12seq −i c:\seq1.txt −j c:\seq2.txt −p blastp −o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

```
                                      1 20
Target Sequence: ATGATGCCGATGATATTAAC
                                      | || ||| |||| |||| |
Identified Sequence:ACGAGGCCAATGACATTAGC
```

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity to an ASPP1, ASPP2, or iASPP protein sequence (which can be used in the disclosed methods) will show increasing percentage identities when assessed by this method, such as at least about 75%, 80%, 85%, 90%, 95%, 98%, or 99% sequence identity.

When aligning short peptides (fewer than around 30 amino acids), the alignment is be performed using the Blast 2 sequences function, employing the PAM30 matrix set to default parameters (open gap 9, extension gap 1 penalties). ASPP1, ASPP2, or iASPP proteins with even greater similarity to the reference sequence will show increasing percentage identities when assessed by this method, such as at least about 60%, 70%, 75%, 80%, 85%, 90%, 95%, 98%, 99% sequence identity. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to an ASPP1, ASPP2, or iASPP sequence determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Short interfering or interrupting RNA (siRNA): Double-stranded RNAs that can induce sequence-specific post-transcriptional gene silencing, thereby decreasing or even inhibiting gene expression. In some examples, siRNA molecules are about 19-23 nucleotides in length, such as at least 19 nucleotides, for example at least 21 or at least 23 nucleotides.

In one example, siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends. The direction of dsRNA processing determines whether a sense or an antisense target RNA can be cleaved by the produced siRNA endonuclease complex. Thus, siRNAs can be used to modulate transcription, for example, by silencing genes, such as HMGN1, HMGN2, or combinations thereof. The effects of siRNAs have been demonstrated in cells from a variety of organisms, including Drosophila, C. elegans, insects, frogs, plants, fungi, mice and humans (for example, WO 02/44321; Gitlin et al., Nature 418:430-4, 2002; Caplen et al., Proc. Natl. Acad. Sci. 98:9742-9747, 2001; and Elbashir et al., Nature 411:494-8, 2001).

Specific binding agent: An agent that binds substantially only to a defined target. For example, a protein-specific binding agent binds substantially only the specified protein and a nucleic acid specific binding agent binds substantially only the specified nucleic acid. In one example, an ASPP2 specific binding agent binds substantially only an ASPP2 protein, while an ASPP1 specific binding agent binds substantially only an ASPP1 protein. The terms “anti-ASPP1 antibodies” and “anti-ASPP2 antibodies” encompasses antibodies specific for an ASPP1 or ASPP2 protein, respectively, as well as immunologically effective portions (“fragments”) thereof. Exemplary antibodies include polyclonal or monoclonal antibodies, humanized antibodies, or chimeric antibodies, as well as any other agent capable of specifically binding to an ASPP1 or ASPP2 protein.

Shorter fragments of antibodies can also serve as specific binding agents. For instance, Fabs, Fvs, and single-chain Fvs (SCFvs) that bind to a specified protein would be specific binding agents. These antibody fragments include: (1) Fab, the fragment containing a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. Methods of making these fragments are routine. For example, construction of Fab expression libraries permits the rapid and easy identification of monoclonal Fab fragments with the desired specificity for an ASPP1, ASPP2, or iASPP protein described herein. Domain antibodies are the smallest part of an antibody (approximately 13 kDa). Examples are disclosed in U.S. Pat. Nos. 6,248,516; 6,291,158; 6,127,197 (all herein incorporated by reference).

Antibodies can also be produced using standard procedures, for example as described in Harlow and Lane (Antibodies: A Laboratory Manual. 1988). For example, polyclonal antibodies can be produced by immunizing a host animal by injection with an ASPP1, ASPP2, or iASPP peptide (or variants, fragments, or fusions thereof). The production of monoclonal antibodies can be accomplished by a variety of methods, such as the hybridoma technique (Kohler and Milstein, Nature 256:495-7, 1975), the human B-cell technique (Kosbor et al., Immunology Today 4:72, 1983), or the EBV-hybridoma technique (Cole et al., in Monoclonal Antibodies and Cancer Therapy, Alan R. Liss, Inc., pp. 77-96, 1983). Additionally, chimeric antibodies can be produced (for example, see Morrison et al., J. Bacteriol. 159:870, 1984; Neuberger et al., Nature 312:604-8, 1984; Takeda et al., Nature 314:452-4, 1985, and PCT International Publication Number WO 92/04381), as well as single-chain antibodies (for example, see U.S. Pat. Nos. 5,476,786; 5,132,405; and 4,946,778) and humanized antibodies in which non-human complementarity determining regions (CDRs) are covalently joined to human FR and/or Fc/pFc′ regions to produce a functional antibody (for example see U.S. Pat. Nos. 4,816,567; 5,225,539; 5,585,089; 5,693,762; and 5,859,205).

The determination that a particular agent binds substantially only to an ASPP1, ASPP2, or iASPP protein can be made using or adapting routine procedures. For example, western blotting can be used to determine that a specific binding agent, such as a mAb, binds substantially only to the protein (Harlow and Lane, Antibodies: A Laboratory Manual. 1988). Other assays include, but are not limited to, competitive and non-competitive homogenous and heterogeneous enzyme-linked immunosorbent assays (ELISA) as symmetrical or asymmetrical direct or indirect detection formats; “sandwich” immunoassays; immunodiffusion assays; in situ immunoassays (for example, using colloidal gold, enzyme or radioisotope labels); agglutination assays; complement fixing assays; immunoelectrophorectic assays; enzyme-linked immunospot assays (ELISPOT); radioallergosorbent tests (RAST); fluorescent tests, such as used in fluorescent microscopy and flow cytometry; Western, grid, dot or tissue blots; dip-stick assays; halogen assays; or antibody arrays (for example, see O'Meara and Tovey, Clin. Rev. Allergy Immunol., 18:341-95, 2000; Sambrook et al., 2001, Appendix 9; Simonnet and Guilloteau, in: Methods of Immunological Analysis, Masseyeff et al. (Eds.), VCH, New York, 1993, pp. 270-388).

On one example, the specificity of ASPP1, ASPP2 or iASPP binding to a binding agent is shown by binding equilibrium constants. In particular examples, targets capable of selectively binding an ASPP1, ASPP2 or iASPP peptide have binding equilibrium constants of at least about 10⁷ M⁻¹, such as at least about 10⁸ M⁻¹, such as at least about 10⁹ M⁻¹.

A specific binding agent also can be labeled for direct detection (see Chapter 9, Harlow and Lane, Antibodies: A Laboratory Manual. 1988). Suitable labels include (but are not limited to) enzymes (such as alkaline phosphatase or horseradish peroxidase), fluorescent labels, colorimetric labels, radioisotopes, chelating agents, dyes, colloidal gold, ligands (such as biotin), and chemiluminescent agents.

Subject: Living multicellular vertebrate organisms, a category which includes both human and veterinary subjects for example, mammals, rodents, and birds.

Therapeutically active molecule: An agent, such as an ASPP1 or ASPP2 protein, nucleic acid molecule, mimetic or agonist thereof, that can increase apoptosis included by p53, p63, or p73, or increase the promoter activity of Bax, as measured by clinical response (for example a decrease in the size of a tumor or a decrease in metastases).

In particular examples, it is an agent, such as an inhibitor of an iASPP protein, nucleic acid molecule such as an antagonist thereof, that can increase apoptosis included by p53 in the presence of ASPP1 or ASPP2, as measured by clinical response (for example a decrease in the size of a tumor or a decrease in metastases).

Therapeutically active molecules can also be made from nucleic acid molecules. Examples of nucleic acid molecule based therapeutically active molecules are a nucleic acid sequence that encodes ASPP1, ASPP2, or iASPP (or fragments that of that encode a peptide that retains the desired biological activity), wherein the nucleic acid sequence is operably linked to a control element such as a promoter. Therapeutically active agents can also include organic or other chemical compounds that mimic the effects of ASPP1, ASPP2, or iASPP peptides.

Therapeutic Amount: The preparations disclosed herein are administered in a therapeutically effective amount, which is an amount of a pharmaceutical preparation that alone, or together with further doses, stimulates the desired response, such as an amount necessary to improve signs or symptoms of a disease. A desired response can be an increase in apoptosis of tumor cells, such as a tumor cell expressing p63 or p63, or a tumor that does not express p53 or expresses a mutant p53. One example of a therapeutic effect is regression of the tumor, lysis of the cells of the tumor, or both. Treatment can involve only slowing the progression of the disease temporarily, but can also include halting or reversing the progression of the disease permanently. For example, in the case of a tumor such as a cancer, treatment can include reducing progression or metastasis of the tumor, or reducing the tumor itself, such as reducing the volume of the tumor. The therapeutically effective amount can include a quantity of ASPP1 or ASPP2 protein, nucleic acid molecule, specific binding agent, mimetic, or agonist sufficient to achieve a desired effect in a subject being treated. In some examples, the therapeutically effective amount includes a quantity of an antagonisit of an iASPP protein or, nucleic acid molecule, such as an antisense or RNAi molecule, sufficient to achieve a desired effect in a subject being treated.

An effective amount of ASPP1 or ASPP2 protein, nucleic acid molecule, specific binding agent, mimetic thereof, or agonist can be administered in a single dose, or in several doses, for example daily, during a course of treatment. However, the effective amount can be dependent on the source applied (for example, ASPP1 peptide isolated from a cellular extract versus a chemically synthesized and purified ASPP1 peptide, or a variant or fragment that may not retain full ASPP1 activity), the subject being treated, the severity and type of the condition being treated, and the manner of administration. For example, a therapeutically effective amount of ASPP1 or ASPP2 protein can vary from about 0.01 mg/kg body weight to about 1 g/kg body weight, such as about 1 mg per subject. Where nucleic acids encoding ASPP1 , ASPP2 or iASPP or variants thereof are employed, doses of between 1 ng and 0.1 mg generally can be formulated and administered according to standard procedures.

The methods disclosed herein have equal application in medical and veterinary settings. Therefore, the general term “subject being treated” is understood to include all animals (such as humans, apes, dogs, cats, horses, and cows) that are in need of an increase in ASPP1 or ASPP2 activity or a decrease in iASPP activity.

Transduced and Transformed: A virus or vector “transduces” or “transfects” a cell when it transfers a nucleic acid molecule into the cell. A cell is “transformed” by a nucleic acid molecule transduced into the cell when the DNA becomes stably replicated by the cell, either by incorporation of the nucleic acid molecule into the cellular genome, or by episomal replication. As used herein, the term transformation encompasses all techniques by which a nucleic acid molecule can be introduced into such a cell, including transfection with viral vectors, transformation with plasmid vectors, and introduction of naked DNA by electroporation, lipofection, and particle gun acceleration.

Transfected: A transfected cell is a cell into which has been introduced a nucleic acid molecule by molecular biology techniques. As used herein, the term transfection encompasses all techniques by which a nucleic acid molecule might be introduced into such a cell, including transfection with viral vectors, transformation with plasmid vectors, and introduction of naked DNA by electroporation, lipofection, and particle gun acceleration.

Transgene: An exogenous nucleic acid sequence supplied by a vector. In one example, a transgene encodes an ASPP1, ASPP2, or iASPP polypeptide.

Tumor: A neoplasm. Includes solid and hematological (or liquid) tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Variants, fragments or fusion proteins: The disclosed ASPP1, ASPP2, or iASPP sequences include variants, fragments, and fusions thereof that retain desired properties, such as the ability of ASPP1 or ASPP2 to increase the apoptotic function of p53, p63, or p73, or the ability of iASPP to decrease the apoptotic function of p53 in the presence of ASPP1 or ASPP2. DNA sequences which encode an ASPP1 or ASPP2 protein or fusion thereof, or a fragment or variant of thereof (for example a fragment or variant having 80%, 90%, 95% or 98% sequence identity to an ASPP1, ASPP2, or iASPP sequence) can be engineered to allow the protein to be expressed in eukaryotic cells or organisms, bacteria, insects, or plants. To obtain expression, the DNA sequence can be altered and operably linked to other regulatory sequences. The final product, which contains the regulatory sequences and the protein, is referred to as a vector. This vector can be introduced into eukaryotic, bacteria, insect, or plant cells. Once inside the cell the vector allows the protein to be produced.

A fusion protein including a protein, such as ASPP1 or ASPP2 (or variants or fragments thereof) linked to other amino acid sequences that do not significantly decrease the desired activity of ASPP1 or ASPP2, for example the characteristic of increasing the apoptotic function of p53, p63, or p73 and increasing the promoter activity of Bax. In one example, the other amino acid sequences are no more than about 10, 20, 30, or 50 amino acid residues in length.

In particular examples, the disclosed nucleic acid molecules and peptides include additions, substitutions, and deletions of one or more nucleotides or amino acids. For example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more additions, substitutions, and deletions can be made to the disclosed molecules, as long as such variant moleues retain the desired biological activity, such as ASP or iASPP activity. For example, a variant ASPP1 or ASPP1 molecule retains one or more of the ability to increase apoptosis, bind p53, p63, or p73, and increase transcriptional activity on BAX promoters.

In particular examples, the disclosed nucleic acid molecules and peptides are fragments of ASPP1, ASPP2 or iASPP. In one example, a fragment of an ASPP1 or ASPP1 molecule is a functional fragment that retains one or more of the ability to increase apoptosis, bind p53, p63, or p73, and increase transcriptional activity on BAX promoters. In other examples, fragments of ASPP1, ASPP2 and iASPP nucleic acid molecules can be used as probes in hybridization blot assays.

One of ordinary skill in the art will appreciate that the DNA can be altered in numerous ways without affecting the biological activity of the encoded protein. For example, PCR can be used to produce variations in a DNA sequence that encodes ASPP1, ASPP2, or iASPP. Such variants can be variants optimized for codon preference in a host cell used to express the protein, or other sequence changes that facilitate expression.

One of ordinary skill in the art can readily determine using the assays described herein and those well known in the art to determine whether a variant, fragment, or fusion is a functional fragment of an ASPP1, ASPP2, or iASPP molecule using no more than routine experimentation. For example, the activity of variants, fragments, or fusions of ASPP1, ASPP2 or iASPP polypeptides can be tested by cloning the nucleic acid molecule encoding the variant, fragment, or fusion ASPP1, ASPP2 or iASPP polypeptide into a bacterial or mammalian expression vector, introducing the vector into an appropriate host cell, expressing the variant, fragment, or fusion ASPP1, ASPP2 or iASPP polypeptide, and testing for a functional capability of the ASPP1, ASPP2 or iASPP polypeptides as disclosed herein. For example, a variant ASP polypeptide can be tested for p53, p63, or p73 binding as disclosed in Examples 3 and 15.

Vector: A nucleic acid molecule as introduced into a host cell, thereby producing a transformed host cell. A vector can include nucleic acid sequences that permit it to replicate in the host cell, such as an origin of replication. A vector can also include one or more therapeutic genes or selectable marker genes and other genetic elements known in the art (such as β-galactosidase, luciferase, alkaline phosphatase, fluorescent proteins). A vector can transduce, transform or infect a cell, thereby causing the cell to express nucleic acid molecules or proteins other than those native to the cell. A vector optionally includes materials to aid in achieving entry of the nucleic acid molecule into the cell, such as a viral particle, liposome, protein coating or the like. Vectors include, but are not limited to, plasmids, phagemids and virus genomes. Viral vectors include, but are not limited to, retroviral and adenoviral vectors.

### ASPP1 and ASPP2 Nucleic Acids and Peptides

Disclosed herein are polypeptides, or part thereof, which include at least one ankyrin repeat, an α helical domain, and an SH3 domain, wherein the polypeptide is capable of stimulating the apoptotic function of p53, p63, p73, or combinations thereof. In some examples, the polypeptide is capable of binding to an antibody, such as a monoclonal antibody, to at least one region of the peptide presented in SEQ ID NO: 2 or 4. In particular examples, the disclosed peptide includes comprises a binding site capable of binding, and thereby associating, with p53, p63, p73, or combinations thereof. In some examples, this association is capable of inducing or enhancing apoptosis.

The disclosed peptides can be of mammalian origin, such as a human peptide. In a particular example, the disclosed polypeptides are represented by the amino acid sequences shown in SEQ ID NO: 2 or 4. However, one skilled in the art will appreciate that variant sequences, such as sequences having one or more deletions, additions, or substitutions (such as 1, 2, 3, 4, 5, 10, or 15 of such modifications), are encompassed by this disclosure as long as such variants retain the ability to increase the apoptotic function of p53, p63, or p73. For example, the disclosed peptides can increase the apoptotic function of p53, p63, or p73 by at least 10%, at least 25%, at least 50%, at least 75%, at least 100%, at least 200%, or even at least 500% as compared to an amount of apoptosis in the absence of the peptide.

Also provided herein are nucleic acid molecules that encode polypeptides, or part thereof, which includes at least one ankyrin repeat, an α helical domain, and a, SH3 domain, wherein the polypeptide is capable of stimulating the apoptotic function of p53, p63, p73, or combinations thereof. In particular examples, the nucleic acid molecules include the sequences shown in SEQ ID NOS: 1 and 3 and fragments thereof such as nucleotides 159-3431 of SEQ ID NO: 1 and nucleotides 256-3642 of SEQ ID NO: 3, sequences which hybridise to SEQ ID NOS: 1 and 3 and encode a peptide capable of stimulating the apoptotic function of p53, p63, p73, as well as nucleic acid sequences which are degenerate as a result of the genetic code. Also disclosed are ASPP nucleic acid molecules, such as ASPP1 or ASPP1 that are part of a vector adapted to facilitate recombinant expression of the polypeptide encoded by the nucleic acid molecule. In a particular example, the vector is an expression vector adapted for eukaryotic gene expression. The vector can include a secretion signal to facilitate purification of the polypeptide. In addition, the vector can include an additional amino acid sequence to facilitate purification of the peptide from a cell or cell culture medium. Such sequences include, but are not limited to, a His-tag sequence that allows the binding of the recombinant polypeptide to a nickel column, or biotin that allows for purification of the peptide on avidin columns.

### iASPP Nucleic Acids and Peptides

Also disclosed herein are peptides, or part thereof, that include at least one ankyrin repeat, and an SH3 domain, wherein the peptide is capable of reducing or inhibiting the p53-apoptotic activity of an ASPP1 or ASPP2 peptide, such as the peptide shown in SEQ ID NO: 6. In some examples, the peptide further includes a proline-rich region. In some examples, the polypeptide is capable of binding to an antibody, such as a monoclonal antibody, to at least one region of the iASPP peptide shown in SEQ ID NO: 6.

The disclosed iASPP peptides can be of mammalian origin, such as a human peptide. In a particular example, the disclosed iASPP peptides are represented by the amino acid sequence shown in SEQ ID NO: 6. However, one skilled in the art will appreciate that variant sequences, such as sequences having one or more deletions, additions, or substitutions (such as 1, 2, 3, 4, 5, 10, or 15 of such modifications), are encompassed by this disclosure as long as such variants retain the ability to reduce the p53-stimulatory activity of an ASPP2 peptide. For example, the disclosed iASPP peptides can reduce the p53-stimulatory activity of an ASPP2 peptide by at least 10%, at least 25%, at least 50%, at least 75%, at least 100%, at least 200%, or even at least 500% as compared to an amount of p53-stimulatory activity of ASPP2 in the absence of the peptide.

Also provided herein are nucleic acid molecules that encode polypeptides, or part thereof, which includes one ankyrin repeat, an SH3 domain, and in some examples also a proline-rich region, wherein the polypeptide is capable of reducing the p53-stimulatory activity of an ASPP2 peptide. In particular examples, the nucleic acid molecule include the sequence shown in SEQ ID NO: 5, sequences which hybridise to SEQ ID NO: 5 and encode a peptide capable of reducing the p53-stimulatory activity of an ASPP2 peptide, as well as nucleic acid sequences which are degenerate as a result of the genetic code. Also disclosed are iASPP nucleic acid molecules that are part of a vector adapted to facilitate recombinant expression of the polypeptide encoded by the nucleic acid molecule. In a particular example, the vector is an expression vector adapted for eukaryotic gene expression. The vector can include a secretion signal to facilitate purification of the polypeptide. In addition, the vector can include an additional amino acid sequence to facilitate purification of the peptide from a cell or cell culture medium.

Methods of producing the disclosed peptides are known in the art. In one example, a peptide is purified from cells that naturally produce the peptide using chromatographic means or immunological recognition. In another example, a cell can be transformed with one or more of the disclosed nucleic acids, such as a nucleic acid encoding ASPP1 or ASPP2, growing said cell in conditions conducive to producing the peptide, then purifying or isolating the peptide from the cell, or its growth environment (such as the medium in which the cell is growing). In other examples, peptides can be synthesized chemically, such as on a peptide synthesizer. Translation of mRNA in cell-free extracts such as the reticulocyte lysate system can also be used to produce a peptide. Other methods of isolating a peptide include, but are not limited to, immunochromatography, HPLC, size-exclusion chromatography, ion-exchange chromatography and immune-affinity chromatography.

Similarly, methods of producing the nucleic acid sequences are known in the art. For example, nucleic acid can be produced in vitro by, for example, polymerase chain reaction (PCR), recombinantly produced by cloning, and synthesized by, for example, chemical synthesis.

### Methods of Treating a Tumor Using ASPP1 or ASPP2 Agents

Disclosed herein are methods that can be used to treat a tumor, such as a tumor in a subject. The method includes administering to a subject a therapeutically effective amount of ASPP1 or ASPP2 proteins, nucleic acids, mimetics thereof, agonists, or combinations thereof, thereby treating the tumor, for example by halting progression of the tumor, by causing regression of the tumor, or retarding growth of the tumor.

In some examples, the disclosed ASPP agents are administered to a subject alone or in combination with one or more other anti-tumor agents, such as a chemotherapeutic agent, agents that act on the tumor neovasculature, or immunomodulators. Exemplary agents that act on tumor neovasculature include combrestatin A4, angiostatin and endostatin. Exemplary immunomodulators include α-interferon, γ-interferon, and tumor necrosis factor alpha (TNFα). The additional agents can be administered before, during or after administration of the ASPP agents. In particular examples, administration of ASPP1 or ASPP2 proteins, nucleic acids, mimetics, or agonists induces apoptosis of the cells of the tumor.

In one example, the expression profile of the tumor is determined prior to administering a therapeutically effective amount of the ASPP agent. For example, a determination can be made as to whether the tumor expresses p63, p73, p53, or mutant p53. Standard molecular biology methods can be used to determine such expression, for example PCR, assaying with labelled hybridization probes, western blotting, and Southern blotting. This allows one, such as a physician, to determine if administration of one or more ASPP agents to the subject will treat the tumor. For example, if the subject is determined to have a tumor that expresses p63 or p73, but no (or little) functional p53, administering a therapeutically effective amount of the ASPP agent will cause apoptosis of the tumor cells. Similarly, if the subject is determined to have a tumor that expresses p63 or p73, and a mutant p53, administering a therapeutically effective amount of the ASPP agent will cause apoptosis of the tumor cells. Exemplary tumors that express mutant p53 include, but are not limited to lung cancers, breast cancers, and leukemias. In addition, if the subject is determined to have a tumor that does not expresses p63 or p73, but expresses p53, administering a therapeutically effective amount of the ASPP agent will cause apoptosis of the tumor cells. However, if the subject is determined to have a tumor that does not express p63, p73, or p53, administering a therapeutically effective amount of the ASP agent will not likely cause apoptosis of the tumor cells.

In some examples, the method also includes monitoring the effect of the therapeutic composition on the tumor. For example, the size of the tumor can be determined, as can the presence of metastases.

### Methods of Treating a Tumor Using iASPP Inhibitors

Disclosed herein are inhibitors of iASPP, such as agents that decrease iASPP expression or activity, and methods of using such agents to treat a tumor. In one example, the iASPP inhibitor is an iASPP antisense nucleic acid molecule, RNAi molecule, ribozyme, or triple helix molecule, such as a molecule that recognizes SEQ ID NO: 5 or a portion thereof. In a particular example, an iASPP antisense nucleic acid molecule recognizes the sense sequence comprising nucleotides—37-536 of iASPP. The method includes administering to a subject a therapeutically effective amount of an iASPP1 inhibitor, thereby treating the tumor, for example by halting progression of the tumor, by causing regression of the tumor, or retarding growth of the tumor.

In some examples, the disclosed ASP agents are administered to a subject alone or in combination with one or more other anti-tumor agents, such as a chemotherapeutic agent, agents that act on the tumor neovasculature, or immunomodulators. In some examples, the method also includes monitoring the effect of the therapeutic composition on the tumor. For example, the size of the tumor can be determined, as can the presence of metastases.

### Methods of Screening

Methods are provided for screening for agents capable of modulating apoptosis, for example by modulating the activity of ASPP1, ASPP2, or iASPP. For example, the disclosure provides methods for identifying agents that increase the activity of ASPP1 or ASPP2, or increase the binding or ASPP1 or ASPP2 to p53, p63 or p73, and thus may increase apoptosis. In addition, the disclosure provides methods for identifying agents that decrease the activity of ASPP1 or ASPP2, or decrease the binding or ASPP1 or ASPP2 to p53, p63 or p73, and thus may decrease apoptosis. Similarly, methods are disclosed for identifying agents that increase the activity of iASPP, and thus may decrease apoptosis, or identifying agents that decrease the activity of iASPP and thus may increase apoptosis.

In one example, the screening method including assaying for compounds that increase or decrease binding between ASPP1 or ASPP2 and p53, p63 or p73. In other examples, the screening method including contacting compounds with a cell that expresses ASPP1 or ASPP2 and p53, p63 or p73 (and optionally iASPP or an inhibitor thereof), and determining the effect of the compound on apoptosis of the cell, and in some examples the effect on Bax promoter activity. Such methods are adaptable to automated, high throughput screenings.

Exemplary assays for screening test agents include, but are not limited to, labeled in vitro protein-protein binding assays, electrophoretic mobility shift assays, immunoassays, and cell-based assays such as two- or three-hybrid screens, expression assays. For example, hybrid screens can be used to rapidly examine the effect of transfected nucleic acids on the intracellular binding of ASPP1, ASPP2 or iASPP polypeptides or fragments thereof to specific intracellular targets. The transfected nucleic acids can encode, for example, combinatorial peptide libraries or antisense molecules. Convenient reagents for such assays, such as GAL4 fusion proteins, are known in the art. An exemplary cell-based assay involves transfecting a cell with a nucleic acid encoding an ASP polypeptide fused to a GAL4 DNA binding domain and a nucleic acid encoding a p53, p63, or p73 domain that interacts with ASP fused to a transcription activation domain such as VP16. The cell also contains a reporter gene operably linked to a gene expression regulatory region, such as one or more GAL4 binding sites. Activation of reporter gene transcription occurs when the ASP and p53 (or p63 or p73) fusion polypeptides bind such that the GAL4 DNA binding domain and the VP16 transcriptional activation domain are brought into proximity to enable transcription of the reporter gene. Agents which modulate a ASP polypeptide mediated cell function are then detected through a change in the expression of reporter gene. Methods for determining changes in the expression of a reporter gene are known in the art.

The ASPP1, ASPP2 or iASPP proteins (or variants, fragments or fusions thereof) used in the screening methods, when not produced by a transfected nucleic acid molecule, are added to an assay mixture as an isolated polypeptide. ASPP1, ASPP2 or iASPP polypeptides can be produced recombinantly or isolated from biological extracts. Full-length or functional fragments of ASP, p53, p63, or p73 can be used, as can mimetics and analogs thereof, as long as the portion, mimetic or analog provides binding affinity and avidity measurable in the assay.

The assay mixture also includes a test agent. In particular examples, a plurality of assay mixtures are run in parallel with different agent concentrations to obtain a different response to the various concentrations. Typically, one of these concentrations serves as a negative control (such as at zero concentration of agent or at a concentration of agent below the limits of assay detection). Test agents encompass numerous chemical classes, such as organic compounds, for example small organic compounds, such as those having a molecular weight of more than 50 yet less than about 2500, such as less than about 1000 and, such as less than about 500. Other exemplary test agents include, but are not limited to cyclic carbon or heterocyclic structure and/or aromatic or polyaromatic structures substituted with one or more of the above-identified functional groups, as well as biomolecules such as peptides, saccharides, fatty acids, sterols, isoprenoids, purines, pyrimidines, derivatives or structural analogs of the above, or combinations thereof and the like.

Test agents can be obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents can be subjected to directed or random chemical modifications such as acylation, alkylation, esterification, and amidification to produce structural analogs of the agents.

Additional reagents can be included in the mixture. Reagents such as salts, buffers, neutral proteins (such as albumin), and detergents, can be used to facilitate optimal protein-protein and/or protein-nucleic acid binding. Such a reagent can also reduce non-specific or background interactions of the reaction components. Other reagents that improve the efficiency of the assay such as protease, inhibitors, nuclease inhibitors, antimicrobial agents, and the like can also be used.

The mixture of assay materials is incubated under conditions whereby, but for the presence of the test agent, the ASPP1, ASPP2 or iASPP peptide specifically binds the cellular binding target. Incubation temperatures typically are between 4° C. and 40° C. Incubation times can be minimized to facilitate rapid, high throughput screening, and such as about 0.1 to 10 hours. After incubation, the presence or absence of specific binding between the ASPP1, ASPP2 or iASPP polypeptide and one or more binding targets (such as p53, p63, or p73) is detected by any convenient method available to the user. For example, in a cell free binding assays, a separation step can be used to separate bound from unbound components. The separation step can be accomplished in a variety of ways. For example, at least one of the components can be immobilized on a solid substrate, from which the unbound components may be easily separated. The solid substrate can be made of a wide variety of materials and in a wide variety of shapes, such as a microtiter plate, microbead, dipstick, or resin particle. Ideally, the substrate provides maximum signal to noise ratios, to minimize background binding.

In one example, separation is achieved by removing a bead or dipstick from a reservoir, emptying or diluting a reservoir such as a microtiter plate well, rinsing a bead, particle, chromatographic column or filter with a wash solution or solvent. The separation step can include multiple rinses or washes. For example, when the solid substrate is a microtiter plate, the wells can be washed several times with a washing solution, which typically includes those components of the incubation mixture that do not participate in specific bindings such as salts, buffer, detergent, non-specific protein. Where the solid substrate is a magnetic bead, the beads can be washed one or more times with a washing solution and isolated using a magnet.

Detection of the presence of absence of ASP-p53, -p63 or -p73 complexes or iASPP complexes can be achieved using any method known in the art. For example, the transcript resulting from a reporter gene transcription assay of ASPP1, ASPP2 or iASPP polypeptide interacting with a target molecule typically encodes a directly or indirectly detectable product (such as β-galactosidase activity, luciferase activity, and the like). For cell free binding assays, one of the components usually includes, or is coupled to, a detectable label. A wide variety of labels can be used, such as those that provide direct detection (such as radioactivity, luminescence, optical or electron density) or indirect detection (such as epitope tag such as the FLAG epitope, enzyme tag such as horseradish peroxidase). The label can be bound to a ASPP1, ASPP2 or iASPP binding partner, or incorporated into the structure of the binding partner.

A variety of methods can be used to detect the label, depending on the nature of the label and other assay components. For example, the label can be detected while bound to the solid substrate or subsequent to separation from the solid substrate. Labels can be directly detected through optical or electron density, radioactive emissions, nonradiative energy transfers or indirectly detected with antibody conjugates, or strepavidin-biotin conjugates. Methods for detecting the labels are well known in the art.

In one example, the screening method including assaying for compounds that increase or decrease apoptosis in the presence of ASPP1 or ASPP2 and p53, p63 or p73. In particular examples, the method includes contacting a cell with a test agent, wherein the cell expresses an ASP protein as well as a p53, p63 or p73 protein. Following incubation, an apoptosis assay is conducted. For example, a decrease in apoptosis is an indication that the test agent decreases apoptosis, and an increase in apoptosis is an indication that the test agent increases apoptosis. The method can further include determining an amount of Bax promoter activity, wherein a decrease in Bax promoter activity is an indication that the test agent decreases apoptosis, and wherein an increase in Bax promoter activity is an indication that the test agent increases apoptosis.

Agent(s) identified by the screening methods disclosed herein are also encompassed within this disclosure. In particular examples, the agent is an agonist which promotes the activity of an ASPP1 or ASPP2 peptide. In other examples, the agent is an antagonist that decreases the activity of an ASPP1 or ASPP2 peptide. In particular examples, the agent is an agonist that promotes the activity of an iASPP1 peptide, or is an antagonist which decreases or inhibits the activity of an iASPP1 peptide.

### Identification of ASP or iASPP Binding Proteins

Phage display can be used to identify peptides that bind to ASP proteins or iASPP proteins. Such binding peptides may increase or decrease the activity of the ASP or iASPP protein, thereby modulating apoptosis. Briefly, a phage library is prepared (for example with m13, fd, or lambda phage), displaying inserts from 4 to about 80 amino acid residues using conventional procedures. The inserts may can, for example, a completely degenerate or biased array. Phage-bearing inserts are selected that bind to the ASP or iASPP polypeptide. This process can be repeated through several cycles of reselection of phage that bind to the ASP or iASPP polypeptide. Repeated rounds lead to enrichment of phage bearing particular sequences. DNA sequence analysis can be conducted to identify the sequences of the expressed polypeptides.

The minimal linear portion of the sequence that binds to the ASP or iASPP polypeptide can be determined. One can repeat the procedure using a biased library containing inserts containing part or all of the minimal linear portion plus one or more additional degenerate residues upstream or downstream thereof. Yeast two-hybrid screening methods also can be used to identify polypeptides that bind to the ASP or iASPP polypeptides. Thus, the ASP and iASPP peptide disclosed herein, including variants, fuisions, and fragments thereof, can be used to screen peptide libraries, including phage display libraries, to identify and select peptide binding partners of the disclosed ASP or iASPP peptides. Such molecules can be used in screening assays, for purification protocols, and for interfering directly with the functioning of ASP or iASPP.

### Transgenic Mammals

The disclosure also includes transgenic non-human mammals, such as non-human mammals having one or more exogenous nucleic acid molecules incorporated in germ line cells and/or somatic cells. Thus a transgenic mammal includes “knockout” animals having a homozygous or heterozygous gene disruption by homologous recombination, animals having episomal or chromosomally incorporated expression vectors. Knockout animals can be prepared by homologous recombination using embryonic stem cells as is well known in the art. The recombination can be facilitated by the cre/lox system or other recombinase systems known to one of ordinary skill in the art. In certain examples, the recombinase system itself is expressed conditionally, for example, in certain tissues or cell types, at certain embryonic or post-embryonic developmental stages, inducibly by the addition of a compound which increases or decreases expression, and the like. In general, the conditional expression vectors used in such systems use a variety of promoters which confer the desired gene expression pattern (such as temporal or spatial). Conditional promoters also can be operably linked to ASPP1, ASPP2 or iASPP nucleic acid molecules to increase expression of these nucleic acid molecules in a regulated or conditional manner.

Trans-acting negative regulators of ASPP1, ASPP2 or iASPP activity or expression also can be operably linked to a conditional promoter as described above. Such trans-acting regulators include antisense nucleic acids molecules, nucleic acid molecules that encode dominant negative molecules, ribozyme molecules specific for ASPP1, ASPP2 or iASPP nucleic acids, and the like. The transgenic non-human animals can be used to determine the biochemical or physiological effects of diagnostics or therapeutics for conditions characterized by increased or decreased ASPP1, ASPP2 or iASPP expression.

### EXAMPLE 1

### Tissue distribution of ASPP1 and ASPP2 mRNA

The tissue distribution of ASPP1 and ASPP2 was determined using standard northern blot hybridization methods.

Both ASPP1 and ASPP2 mRNA were expressed in all the human tissues tested (including brain, heart, skeletal muscle, colon, thymus, spleen, kidney, liver, placenta, lung leukocyte, and small GI) with a single transcript at the size of 5.5 to 5 kb respectively. However, the expression level of ASPP1 and ASPP2 varied. The highest expression levels of ASPP1 and ASP2 were detected in heart, skeletal muscle and kidney. Interestingly, there is a small difference between the expression pattern between ASPP1 and ASPP2. For ASPP1, the highest expression level is in heart, significantly higher than that seen in the kidney and the skeletal muscles. In contrast the expression level of ASPP2 in heart, skeletal muscle and kidney is similar. In addition a relatively high level expression of ASPP1 was also observed in human liver tissues.

### EXAMPLE 2

### ASPP2 Antibody Production

This example describes methods used to determine the tissue distribution of ASPP2 proteins using standard western blot methods.

A GST-fusion protein was used to generate antibodies to ASPP2 as follows. The coding region spanning amino acids 691-1128 of ASPP2 (amino acids 691-1128 of SEQ ID NO: 4) was subcloned into the EcoR1 site of the bacterial expression plasmid pGEX 2TK. A 74 kDa GST-ASPP2 (691-1128) protein was produced and used to immunise rabbits (Eurogentec, Belgium) and mice. The immunised serum derived from the rabbits and the mice were tested using the cell lysates of Saos-2 cells transfected with an expression plasmid of ASPP2 fragment, pCMV Bam neo ASPP2/53BP2 (600-1128). The plasmid was constructed by inserting a PCR fragment of ASPP2 containing the epitope tag of 9E10 at the BamH1 restriction site. Using the Saos-2 lysate transfected with ASPP2 expression plasmid pCMV Bam neo ASPP2/53BP2 (600-1128) or the control vector, the specificity of the rabbit polyclonal antibody pAbASPP2/77 and the mouse monoclonal antibodies DX54-10 and DX54-7 was confirmed. The mouse monoclonal antibody DX54.10 did not cross react with GST protein and recognized transfected ASPP2 expression proteins in Saos-2 cells. DX54.10 only recognized transfected ASPP2 proteins and GST-ASPP2 protein, but not GST-p27 fusion protein, and is therefore specific to ASPP2.

The DX54.10 monoclonal antibody was used to determine the expression of endogenous ASPP2. To ensure that the reactive band to the antibody was endogenous ASPP2, the antiASPP2 monoclonal antibody DX54.10 supernatant was treated with either GST protein attached to glutathione beads or GST-53BP2 (691-1128) protein attached to glutathione beads. The beads were incubated with the supernatant for one hour on a rotating wheel, and the beads subsequently recovered and discarded. Beads were replaced with fresh beads a total of three times.

Transfected ASPP2/53BP2 fragment (600-1128) and a specific protein band were recognized by the antibody derived from the supernatant incubated with the GST beads but not the ones incubated with GST-ASPP2 beads. These results demonstrate that the recognized protein in the total cell lysates derived from 293 cells and Tero cells were endogenous ASPP2 and the monoclonal antibody DX54-10 was specific to this protein.

### EXAMPLE 3

### ASPP2 and p53 Interact in vivo

This example describes methods used to demonstrate that p53 and ASPP2 interact in vivo.

Expression plasmids encoding p53 and bBP2 were transfected into Saos-2 cells and an immunoprecipitation was performed using the antiASPP2 antibody DX54.10 (Example 2) or a control antibody pAb423 (an antibody to SV40 large T-antigen). Western blot analysis of the immunocomplexes of p53 and ASPP2 demonstrated that these proteins interact in vivo. This interaction was specific because the control antibody did not immunoprecipitate either p53 or ASPP2.

Although there were differences in the migration of endogenous ASPP2 and the transfected ASPP2 (also known as bBP2(123-1128)) proteins on SDS PAGE, this is likely due to the fact that the original sequence of bBP2 (Naumovski and Cleary, Mol. Cell. Biol. 16:3884-92, 1996) shows two potential ATG codons at nucleotide position 571 and 757. The 757 codon was shown to be the preferred start site by in vitro coupled transcription-translation. This predicts a protein of 1005 amino acid residues in size. Therefore an expression plasmid of 53BP2/bBP2 was constructed using the nucleotide 757 as start site (Naumovski and Cleary, Mol. Cell. Biol. 16:3884-92, 1996). However based on the observations described herein, the actual protein translation start site is not 757 codon in vivo.

Using the 5′ end of bBP2 sequence to perform a BLAST search, it was observed that the sequence of bBP2 at 412 to 543 bp has high homology to vector sequence (EMBO entry of bBP2/53BP2). This region of ASPP2/bBP2 plasmid was re-sequenced and it was observed that this region of sequence does not exist in the plasmid sequence. Since there was a stop codon within the region of 412-543 bp of bBP2 sequence in the database, the start site of ASPP2 is upstream of 757. By comparing with the part of the mouse ASPP2 (obtained by screening the cDNA library with the human ASPP2 cDNA), the start site for ASPP2 is likely at 256 bp of the new ASPP2 cDNA sequence. This would make the ASPP2 protein 1128 amino acids long, thereby accounting for the unexpectedly large endogenous protein.

To investigate this further, 53BP2/bBP2 cDNA that contains both ATG start sites (256 and 757) was subcloned into a mammalian expression plasmid pcDNA3. The resulting plasmid, pcDNA3-ASPP2/53BP2(1-1128) was transfected into Saos-2 cells and the expression of both endogenous and exogenous ASPP2 detected by antiASPP2 antibody DX54-10. The ASPP2 expressed from pcDNA3-ASPP2(1-1128) migrated at the same molecular weight as that of endogenous ASPP2. Based on this result, it is concluded that endogenous ASPP2 uses the first ATG and the full length ASPP2 should consist of 1128 amino acids.

Based on these results, the clone names corresponding to the actual sequences themselves were clarified. The name ASPP2 is used herein to represent the full length protein which contains 1128 amino acids, while the term ASPP2/bBP2 and ASPP2/53BP2 are used to represent the proteins containing 123-1128 and 600-1128 amino acids respectively.

In addition to endogenous ASPP2, ASPP2/bBP2 also interacted with p53 in vivo.

### EXAMPLE 4

### Effect of ASPP1 and ASPP2 on p53 Transactivation

p53 is a transcription factor which transactivates many target genes including mdm-2, Bax and cyclin G. In contrast, ASPP2/53BP2 was originally isolated as an inhibitor of p53 because it inhibited the DNA binding activity of p53 in vitro by binding to the central DNA binding region of p53 (Iwabuchi et al., Oncogene 8:1693-6, 1993). In addition, ASPP2/bBP2 confers growth suppression rather than promoting activity (Naumovski and Cleary, Mol. Cell. Biol. 16:3884-92, 1996). However, these previous observations could be because the original clone of ASPP2/53BP2 only contains the C-terminal portion of the protein. Therefore, full length ASPP2 protein could have a different effect on p53 from its C-terminal fragment ASPP2/53BP2.

To demonstrate the effect of ASP family members on the activities of p53, p53-dependent transcriptional activity was determined in transient reporter assays. Cells null for p53 (Saos-2) were transfected with five p53 reporter plasmids: mdm-2, Bax, cyclin G and p21Waf-1 (all derived from the promoters of p53 target genes), and PG, a synthetic promoter construct linked to the expression of the luciferase gene. The amount of luciferase expression was determined as previously described in Samuels-Lev et al. (Mol. Cell 8:781-94, 2001, herein incorporated by reference).

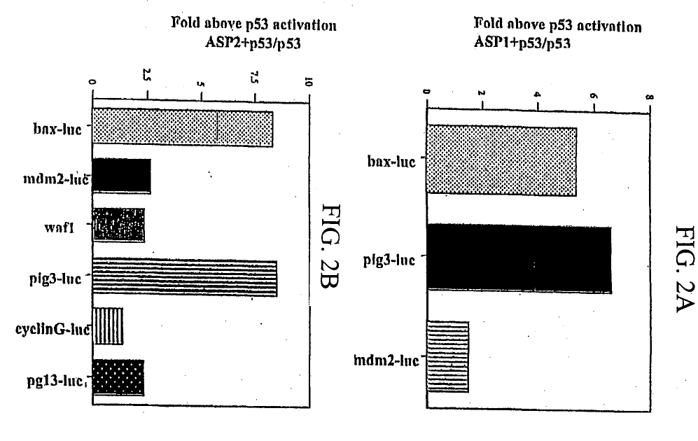

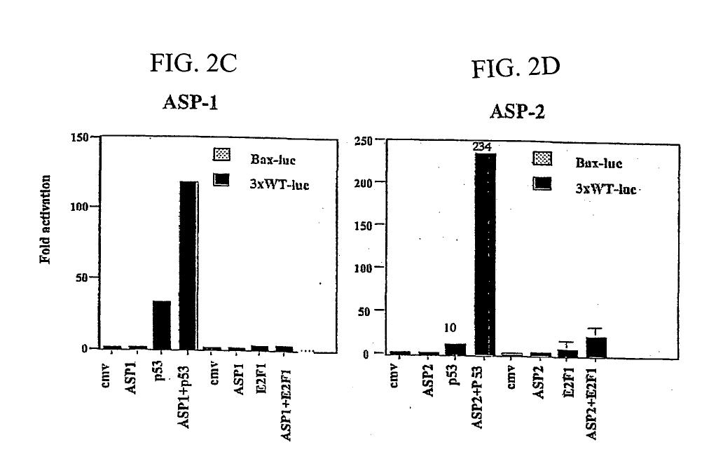

The known p53 binding sites are divided into two groups. Bax-like sites are usually weak for p53 transcription stimulation while the mdm2-like sites can be stimulated by p53 very effectively. As shown in FIGS. 2A and 2B, co-expression of ASPP1 or ASPP2 together with p53 resulted in a 10-50 fold stimulation of the Bax promoter. In contrast, co-expression of either ASPP1 or ASPP2 with p53 only showed a very modest stimulation of the promoter activity of mdm2 and cyclin G. ASPP2/53BP2 failed to stimulate mdm2 and cyclin G promoters while a slight stimulation on p21waf1 and PG synthetic promoters was observed.

The ability of ASPP2/53BP2 to stimulate the promoter activity of Bax but not mdm2 demonstrates for the first time that the promoter specificity of p53 can be regulated in cells. Since Bax is one of the p53 target genes that is pro-apoptotic, it was determined whether ASP family members can specifically stimulate the transactivation of other p53 target genes involved in promoting apoptosis, such as PIG-3. Using the transient transfection reporter assays described above, it was shown that both ASPP1 and ASPP2 specifically stimulated the promoter activity of PIG-3.

The transactivation function of p53 can be co-activated by a general transcription co-activator p300/CBP. To determine whether the ASP family members act like the p300/CBP-like protein that is not specific to p53 and can stimulate a large number of transcription factors, the following methods were used. Like p53, the transactivation function of E2F1 can be stimulated by the co-expression of p300/CBP. However, the co-expression of ASPP1 or ASPP2 with E2F1 failed to stimulate its transactivation function on a few known reporter promoters, including cyclin A, b-myb and the synthetic promoter 3×wt (FIGS. 2C and 2D). This result indicates that ASPP1 and ASPP2 stimulate the transactivation function of p53 specifically. Since the general transcription co-activators p300/CBP can bind to and stimulate the transcriptional activity of both p53 and E2F1, this result also indicates that both ASPP1 and ASPP2 can stimulate the transactivation function of p53 independently of p300/CBP.

### EXAMPLE 5

### Effect of ASPP2 Fragments on the Transactivation Function of p53

As shown in Example 4, co-expression of ASP can specifically stimulate the transactivation function of p53. Using the methods described in Example 4, a minimal region of ASP sufficient for such activity was identified.

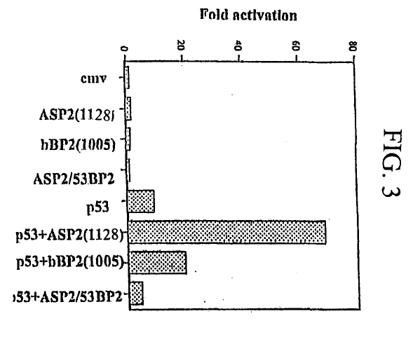

Three different versions of ASPP2 was tested for their effects on the transactivation function of p53. Co-expression of full length ASPP2 (1128 amino acids) further stimulated the transactivation function of p53 about 7 fold. However, under the same conditions, co-expression of ASPP2/bBP2 (1005 amino acids) only stimulated the transactivation function of p53 about 2-fold, and co-expression of ASPP2/53BP2 (amino acids 600-1128) reduced the transactivation function by about 50% (FIG. 3). Failure to stimulate the transactivation function of p53 by ASPP2/bBP2 was not due to the lack of expression.

Therefore, ASPP2/bBP2 which lacks the first 123 amino acids of ASPP2 failed to significantly stimulate the transactivation function of p53. These results indicate that full-length protein (1-1128 aa) is needed for ASPP2 to significantly enhance the transactivation function of p53 (although some transactivation function was observed with only 1005 amino acids). The reduced transactivation function of p53 by ASPP2/53BP2 indicates that ASPP2/53BP2 can act as a dominant negative mutant to inhibit the action of endogenous ASPP2 on p53.

### EXAMPLE 6

### ASPP1 and ASPP2 Synergize with p53, p63 and p73 to Induce Apoptosis

As shown in the Examples above, ASPP1 and ASPP2 can specifically stimulate the transactivation function of p53 on the promoters of Bax and PIG-3. This example describes methods used to demonstrate that co-expression of ASPP1 or ASPP2 with p53 synergizes with p53 to induce apoptosis, and to demonstrate that ASPP1 and ASPP2 have little effect on the apoptotic function of Bax itself.

Saos-2 cells, which are null for p53 and also express a relatively low level of ASPP2, were transfected with vectors encoding full-length ASPP1 or ASPP2, alone or together with p53. The amount of p53 used was determined by titration so that it about 17% of transfected cells to undergo apoptosis. Apoptosis was identified by the expression of the co-transfected cell surface marker CD20, as described in Samuels-Lev et al. (Mol. Cell 8:781-94, 2001, herein incorporated by reference).

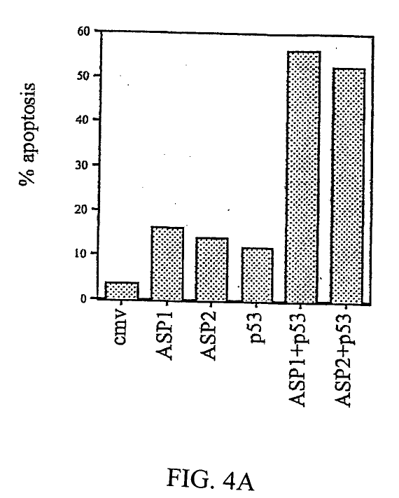

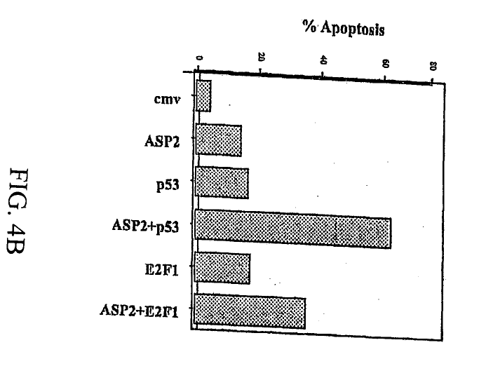

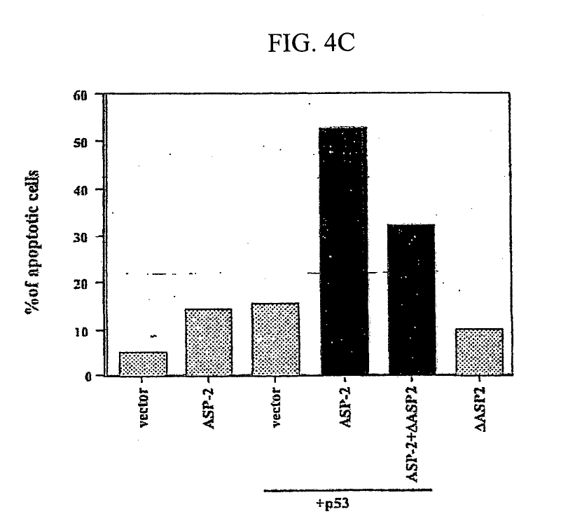

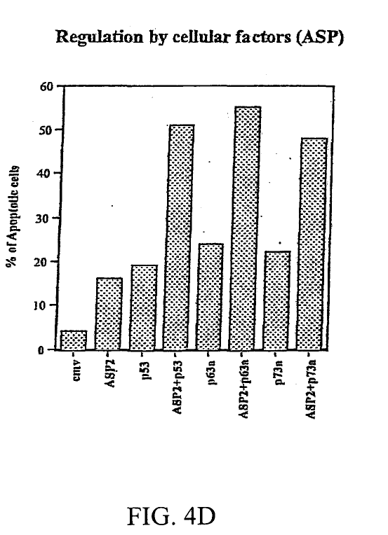

Expression of ASPP1 or ASPP2 alone resulted in a lower level of apoptosis, consistent with the observation that either ASPP1 or ASPP2 alone could enhance Bax promoter activity slightly, possibly due to the effect of ASPP1 and ASPP2 on p73 and p63. Co-expression of p53 with ASPP1 or ASPP2 however resulted in a significant increase in the number of cells that die of apoptosis. Approximately 50% of the transfected cells now die of apoptosis (FIG. 4A). This synergistic effect in enhancing apoptosis was specific to p53 since co-expression of either ASPP1 or ASPP2 with E2F1 resulted in only an additive increase in the percentage of cells that die of apoptosis (FIG. 4B).

The ASPP2 mutant, ASPP2/53BP2, was used to demonstrate that ASP can stimulate the apoptotic function of p53 by enhancing the transactivation function of p53. ASPP2/53BP2 inhibited ASPP2 stimulation of p53 transactivation function of p53 of the Bax promoter. When ASPP2 and p53 were co-expressed 50% of the cells were apoptotic. However when p53, ASPP2 and ASPP2/53BP2 were all co-expressed, only 30% of cells were apoptotic. Thus, ASPP2 can only enhance the apoptotic function of p53 by increasing its transactivation function (FIG. 4C).

The effect of ASP on the apoptotic function of p53 family members, p73 and p63 is shown in FIG. 4D. The co-expression of either ASPP1 or ASPP2 enhanced the apoptotic function of all the members of p53 family. These results indicate that the ASP family is a novel tumour suppressor family.

### EXAMPLE 7

### Regulation of the Pro-Apoptotic Function of ASP by iASPP

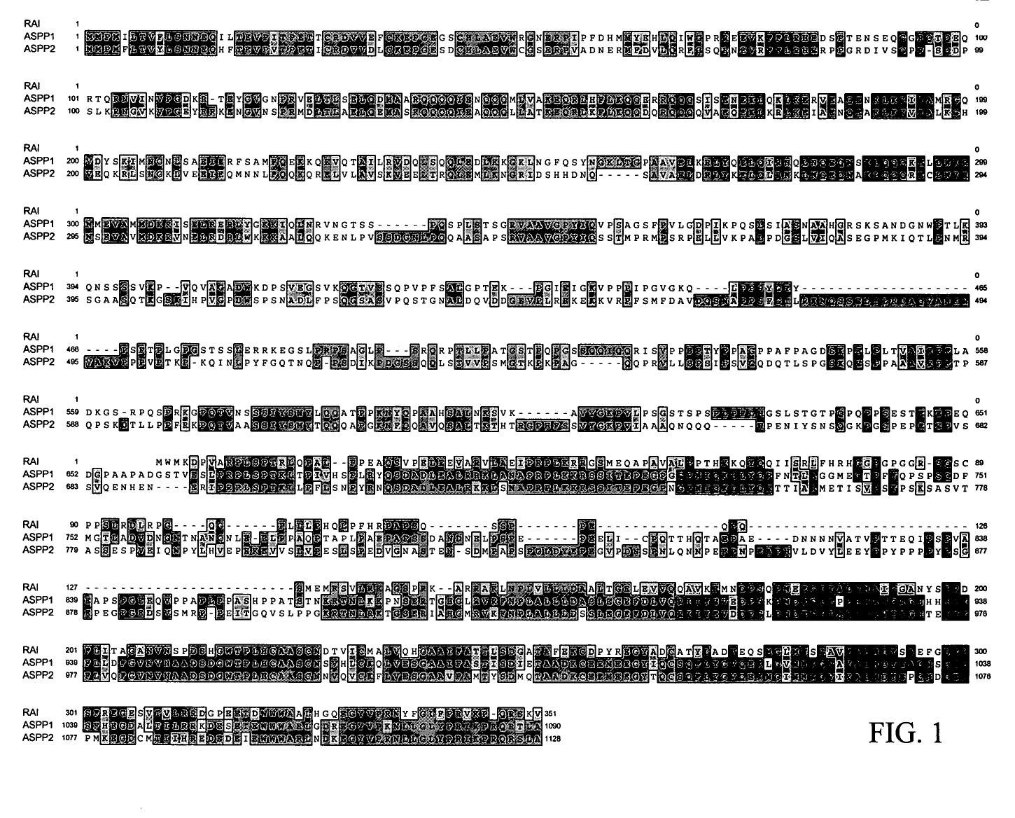

reL Associated Inhibitor (RAI) is a p65 rel A binding protein containing 315 amino acids that has sequence homology to the C-terminal half of ASPP1 and ASPP2 (FIG. 1 and SEQ ID NO: 6). RAI is similar to the ASPP2 mutant, 53BP2/ASPP2(600-1128). For example, although RAI does not have the α-helical domain of ASPP1 or ASPP2, it does contain the proline rich region, the ankryin repeats and the SH3 domain. The p53-contact residues of ASPP2 are also conserved in RAI.

To investigate the activity of RAI, which is referred to herein as iASPP (Inhibitor of Apoptosis Stimulating Proteins), the coding sequence of RAI was cloned into a mammalian expression vector pcDNA3. A peptide (RLQPALPPEAQSVPELEE, amino acids 15-32 of SEQ ID NO: 6) found in iASPP which does not have sequence similarity to ASPP1 and ASPP2 was synthesized. A mouse antibody specific to this unique iASPP peptide did not cross react with either ASPP1 or ASPP2.

Saos2 cells were transfected with either vector alone, p53 (5 μg), full-length iASPP (10 μg) or p53+iASPP and then incubated for 16 hours. The cells were lysed in NP40 lysis buffer and 1000 μg of lysate immunoprecipitated with antibodies to iASPP bound to Protein G beads. The presence of p53 was detected by western blotting of the immunocomplexes using rabbit polyclonal p53 antibody CM1. It was observed that iASPP interacted with p53.

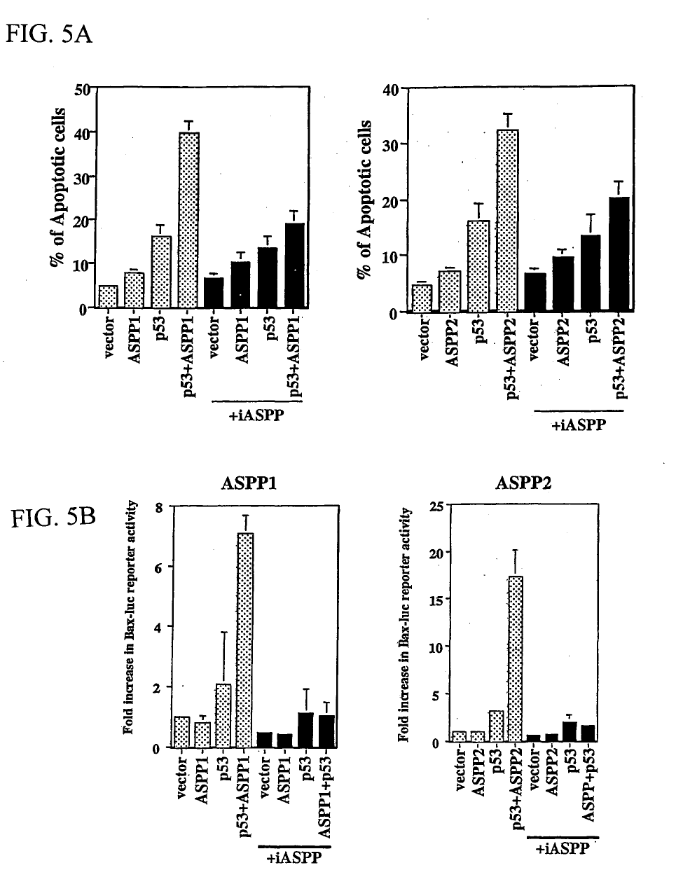

Using the methods described in Example 6, the effect of iASPP on induction of p53 induced apoptosis was determined. Like the ASPP2 mutant 53BP2/ASPP2 (600-1128), expression of iASPP did not induce apoptosis on its own. When iASPP was co-expressed with p53, it had a small inhibitory effect on the apoptotic function of p53. The most significant effect of iASPP on the apoptotic function of p53 was observed when ASPP1 or ASPP2 were co-expressed. Co-expression of iASPP decreased or inhibited the enhanced apoptotic function of p53 effected by ASPP1 and ASPP2 (FIG. 5A). Similarly, co-expression of iASPP together with ASPP1 or ASPP2 decreased or eliminated the ability of both ASPP1 and ASPP2 to stimulate the transactivation function of p53 on the Bax promoter (FIG. 5B).

To determine the effect of iASPP expression on p53 or ASP expression, the following methods were used. Saos2 cells were transfected with either ASPP1 (8 μg) or ASPP2 (4 μg), iASPP (5 μg) and p53 (50 ng). Lysates (40 μl) were run on a 10% gel and ASPP1 was detected with V5 antibody, ASPP2 with DX.5410, iASPP with mouse anti iASPP antibody, p53 with DO1 and PCNA with anti-PCNA antibody. The co-expression of iASPP did not significantly alter the expression levels of either p53 or ASP.

These results demonstrate that in vivo the pro-apoptotic function of ASPP1 and ASPP2 may be regulated by the natural inhibitor iASPP. Thus, the balance between the expression levels of ASPP1, ASPP2 and iASPP may influence cell fate.

### EXAMPLE 8

### Effect of ASP on p53 Mutants

Some apoptotic-defective mutants of p53 can transactivate the promoters of many p53 target genes including mdm2 and p21waf1, but not the pro-apoptotic genes such as Bax, PIG-3 and IGF-BP3. The mutations of p53 at residue 181 (181L and 181C) have been reported in many human tumour types including breast carcinoma and cervical cancer. The residue 181 of p53 is a contact site within p53 for 53BP2 but this residue was not a contact site for DNA. In addition, previous studies have shown that both 181L and 181C can bind to DNA and transactivate many promoters of p53 target genes such as mdm2 and p21waf1. However, both mutants have reduced ability to induce apoptosis or suppress transformation.

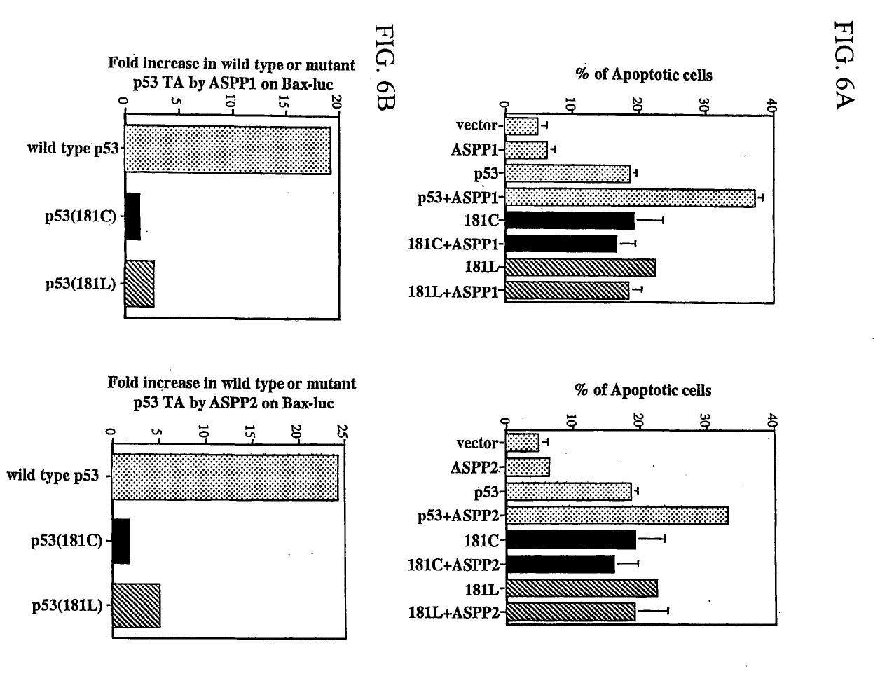

To determine the effect of ASP on the apoptotic function of the p53 tumour-derived mutants 181L and 181C, the following methods were used. Saos-2 cells were transfected with vectors encoding p53 (1 μg/10 cm dish), p53181C (1.6 μg/10 cm dish) or p53181L (2 μg/10 cm dish) in the presence or absence of either ASPP1 (10 μg/10 cm dish) or ASPP2 (10 μg/10 cm dish). The number of apoptotic cells was determined using the methods described in Example 6. As shown in FIG. 6A, co-expression of ASPP1 or ASPP2 failed to enhance the apoptotic function of either p53 mutant, even though within the same experiments, the co-expression of ASP enhanced the apoptotic function of wild type p53 significantly.

The effects of ASP on the transactivation function of the p53 mutants were determined as follows. Saos-2 cells were transfected with ASPP1 or ASPP2 (8 and 4 μg, respectively), and wild type p53 (50-75 ng) or a mutant p53 (p53181C (50 ng) or p53181L (50 ng)), along with a Bax-luc reporter vector. The amount of transactivation function was determined as described in Example 4. The fold activation was obtained by the activity of the various p53 constructs in the presence of ASPP1 or ASPP2 over the activity of the promoter in the presence of the various p53 constructs alone.

Consistent with the observation that mutation of residue 181 can impair the ability of ASP to activate the apoptotic function of p53, co-expression of ASPP1 or ASPP2 was unable to stimulate the transactivation function of the mutant p53, 181C on the Bax gene promoter (FIG. 6B). The effect of ASP on the p53 mutant 181L was similar (FIG. 6B). The inability of ASP to stimulate the activities of p53 mutants was not due to the lack of protein expression (determined by western blotting using 40 μl of the respective transactivation lysates and detection with anti p53 (DO1), anti ASPP2 (DX.5410), and anti V5 ASPP1).

These results indicate that the failure of ASPP1 and ASPP2 to stimulate the transactivation function of the two p53 mutants on pro-apoptotic genes may explain why these two mutant p53 molecules are defective in inducing apoptosis. Furthermore, these results demonstrate the importance of the co-activation function of ASP on the tumour suppression function of p53.

### EXAMPLE 9

### Expression of ASPP1, ASPP2 and iASPP in Breast Carcinoma

All four of the identified 53BP2 contact residues on p53 are mutated in human tumours. In addition, the down-regulation of ASPP2 expression has been found in one case of highly malignant human breast carcinoma in a gene array analysis. This example describes the results from semi-quantitative RT-PCR used to demonstrate that expression levels of ASPP1 and ASPP2 are down-regulated in human tumours expressing wild type p53.

The expression levels of ASPP1 and ASPP2 were determined in a panel of paired normal and tumour RNA samples derived from 40 breast cancer patients. All 40 of the breast carcinomas express wild type p53. The expression levels of ASPP1 and ASPP2 were frequently down regulated in human breast carcinomas (Table 1). Among the 40 carcinoma samples, 24 expressed ASPP1 and 9 expressed ASPP2. In addition, 8/9 tumours with reduced expression of ASPP2 also had reduced ASPP1 expression. This expression pattern indicates that the selective pressure of down regulating the expression of ASPP1 is higher than that of ASPP2. This is consistent with the fact that in the 40 breast carcinomas tested, the frequency of significantly reduced (greater than 75% reduction in the signal) or lack of expression of ASPP1 was higher than that detected for ASPP2, 60% and 22.5% respectively. Since the results were obtained by comparing the expression levels of ASPP1 and ASPP2 between normal tissue and carcinomas derived from the same individuals, there is a selective advantage for the tumour cells to lose the expression of ASPP1 and ASPP2.

These results agree with the results shown in Example 8 that the ASP-binding-impaired-p53 mutants, 181L and 181C, cannot induce apoptosis efficiently even in the presence of ASP. Therefore, it appears that the ASP family of proteins have a tumour suppressing role in human breast carcinomas.

In contrast to ASPP1 and ASPP2, the expression level of iASPP was generally low in the normal and human breast tumour tissue samples tested. However, overexpression of iASPP was detected in 8 of the tumour tissues compared to their normal paired controls (Table 1). There was also a correlation between the normal expression of ASPP1 and ASPP2 with the overexpression of iASPP. Seven of the iASPP overexpressing tumours did not have any detectable down regulation of ASPP1 and ASPP2 expression (Table 1). These results indicate that iASPP is an inhibitor of ASPP1 and ASPP2 in vivo.

TABLE 1
mRNA expression of ASP in wild type p53 expressing
human breast tumor samples (grade I and II)

| Tumor | ASP1 | ASP2 | I-ASP |
|---|---|---|---|
| 1 | ↓ | + | − |
| 2 | ↓ | + | − |
| 3 | ↓ | + | − |
| 4 | ↓ | + | − |
| 5 | ↓ | + | − |
| 6 | ↓ | + | − |
| 7 | ↓ | ↓ | − |
| 8 | + | + | ↑ |
| 9 | ↓ | ↓ | − |
| 10 | ↓ | ↓ | − |
| 11 | ↓ | + | − |
| 12 | ↓ | ↓ | − |
| 13 | ↓ | ↓ | − |
| 14 | ↓ | + | − |
| 15 | ↓ | ↓ | − |
| 16 | ↓ | ↓ | − |
| 17 | + | + | ↑ |
| 18 | ↓ | + | − |
| 19 | ↓ | + | − |
| 20 | + | + | ↑ |
| 21 | + | + | − |
| 22 | + | + | − |
| 23 | ↓ | + | − |
| 24 | + | + | − |
| 25 | + | + | ↑ |
| 26 | + | ↓ | − |
| 27 | ↓ | ↓ | − |
| 28 | + | + | ↑ |
| 29 | + | + | − |
| 30 | ↓ | + | − |
| 31 | + | + | − |
| 32 | + | + | ↑ |
| 33 | ↓ | + | − |
| 34 | ↓ | + | − |
| 35 | + | + | − |
| 36 | + | + | − |
| 37 | + | + | − |
| 38 | + | + | ↑ |
| 39 | ↓ | + | − |
| 40 | ↓ | + | ↑ |

### EXAMPLE 10

### Endogenous ASPP1 and ASPP2 Regulate the Apoptotic Function of Endogenous p53 in Response to DNA Damage

This example describes method used to demonstrate the role of ASP family members in regulating apoptosis induced by endogenous p53. Plasmids expressing ASPP1 or ASPP2 proteins were transfected into the cell lines U2OS and MCF7 that express wild-type p53, together with a cell surface marker CD20. The transfected cells were gated, and the apoptotic cells identified by FACS as described in Example 6.

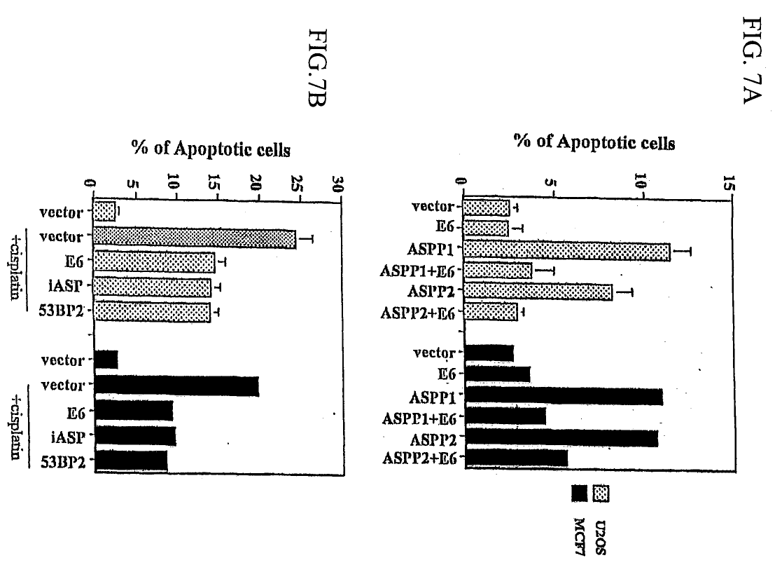

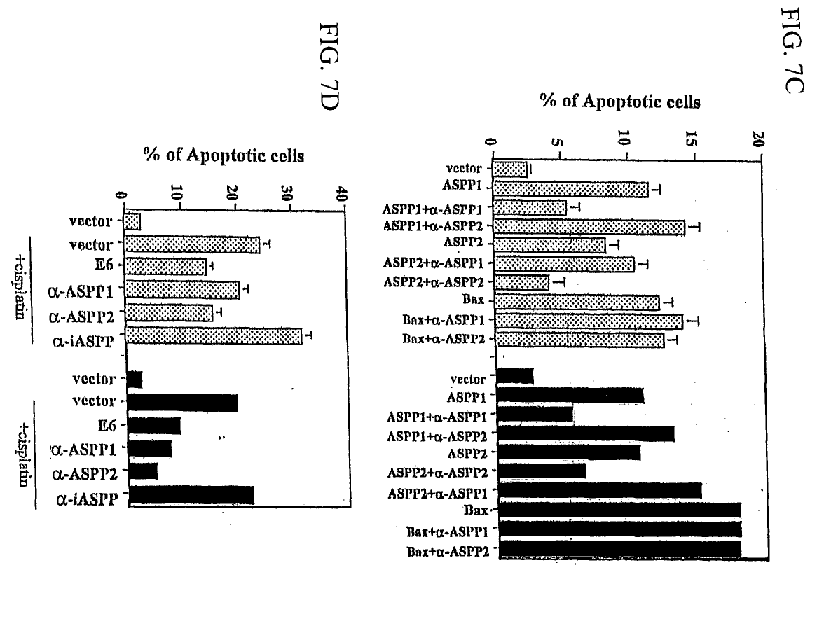

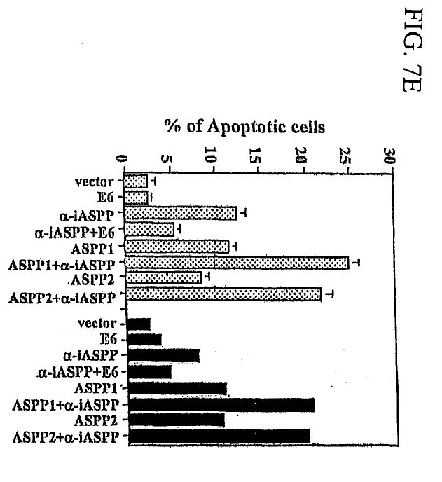

When expressed in these cells, ASPP1 and ASPP2 induced apoptosis (FIG. 7A). The viral oncoprotein E6, which is derived from human papilloma virus and which can bind and specifically target p53 for degradation, inhibited the apoptosis induced by ASPP1 or ASPP2, demonstrating that ASPP1 and ASPP2 can induce p53-dependent apoptosis.

The dominant negative function of 53BP2 and iASPP in inhibiting apoptosis induced by endogenous p53 in response to DNA damage was demonstrated as follows. Before exposure to cisplatin (5 and 3 μg/ml respectively), U2OS and MCF7 cells were transfected with plasmids encoding HPV16 E6, iASPP, or 53BP2. Thirty hours later, cells were harvested and analysed as above. As shown in FIG. 7B, treatment with cisplatin induced over 20% of the transfected cells to die of apoptosis. The expression of E6 reduced the percentage of apoptotic cells to below 15% indicating that cisplatin induces p53-dependent apoptosis in U2OS cells. In agreement with this, expression of iASPP or 53BP2 inhibited cisplatin-induced apoptosis to a similar extent as E6. Therefore, the apoptotic function of endogenous p53 can be regulated by the expression of ASP family members.

To demonstrate further that endogenous ASP family members participate in regulating the apoptotic function of p53, an antisense approach was used. Fragments from the 5′ ends of ASPP1, ASPP2 and iASPP cDNA were cloned into a mammalian expression vector in an antisense orientation and their ability to inhibit the protein synthesis determined in vitro. The antisense nucleic acid molecules were amplified by PCR on the respective plasmid clones using primers spanning the following nucleotide regions (relative to the initial ATG): −74 to 923; −253 to 839 and −37 to 536 for ASPP1, ASPP2 and iASPP respectively. The amplified segments were purified with the QIAquick PCR purification kit (QIAGEN) and ligated in the pcDNA3.1/V5-His TOPO vector (Invitrogen) according to the manufacturer's instructions.

Expression of antisense ASPP1 only inhibited apoptosis induced by ASPP1 but not by ASPP2. Similarly, expression of antisense ASPP2 only inhibited apoptosis induced by ASPP2 but not ASPP1. The specific effect of antisense ASPP1 and ASPP2 was supported by the observation that co-expression of antisense ASPP1 or ASPP2 did not influence apoptosis mediated by Bax under the same conditions (FIG. 7C).

To further demonstrate the role of endogenous ASPP1 and ASPP2 in regulating the apoptotic function of endogenous p53 in response to DNA damage, the following methods were used. U2OS and MCF-7 cells were transfected with the various expression plasmids prior to the treatment with cisplatin and analyzed using FACS as described above. FACS analysis showed that around 20-30% of control transfected cells undergo apoptosis. Expression of E6 reduced the percentage of apoptotic cells to half, indicating that cisplatin can induce apoptosis through both p53 dependent and independent pathways in these cells. Expression of antisense RNA of ASPP1 or ASPP2 inhibited cisplatin-induced apoptosis to the same extent as E6 (FIG. 7D), similar to the effects observed with 53BP2 and iASPP. This indicates that endogenous ASPP1 and ASPP2 participate in regulating the apoptotic function of p53 in response to DNA damage.

The stimulatory effect of the endogenous ASPP1 and ASPP2 on p53 induced apoptosis in response to cisplatin may be under-estimated due to high levels of iASPP detected in these cells, which could prevent ASPP1 and ASPP2 from enhancing the apoptotic function of p53. To determine the anti-apoptotic role of iASPP, both U2OS and MCF-7 cells were transfected with antisense iASPP and cells analyzed as described above. Antisense iASPP induced p53-dependent apoptosis that was abrogated by the co-expression of E6. Removal of the anti-apoptotic function of iASPP by antisense iASPP also enhanced the apoptotic function of ASPP1 and ASPP2 (FIG. 7E). Unlike antisense ASPP1 and ASPP2, expression of antisense iASPP did not inhibit cisplatin-induced apoptosis. A small increase in apoptotic cells was consistently detected (FIG. 7D). These results demonstrate that ASPP1 and ASPP2 specifically stimulate the apoptotic function of p53 in vivo. Therefore, iASPP functions as an inhibitor of ASP and can reduce or inhibit apoptosis induced by endogenous p53.

### EXAMPLE 11

### Ikb Reduces p53-induced Apoptosis and p53 Transactivation Function in the Presence of ASPP1 or ASPP2

p53 and p65RelA of NF kappaB participate in regulating apoptosis in response to stress. However, little is known about how these two apoptotic pathways can work together in vivo. It is known that p53 can induce the DNA binding activity of p65 Rel A, and that Ikb, the inhibitor of p65 Rel A, can inhibit the apoptotic function of p53. Both ASPP2 and iASPP interact with p65 rel A, a component of NF-kappaB, in a yeast hybrid assay. iASPP can also inhibit the transactivation function of p65, although less effectively than Ikb. The region involved in ASPP2 and iASPP interacts with rel A p65 is very similar as that for p53. Therefore, it is possible that there might be some competition between p53 and p65 rel A to interact with ASPP2 and iASPP.

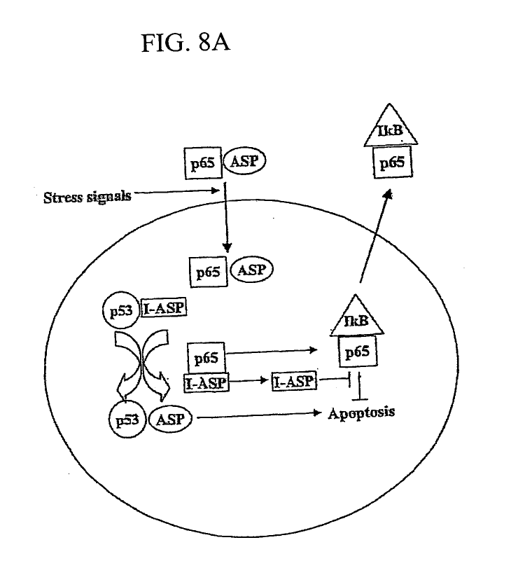

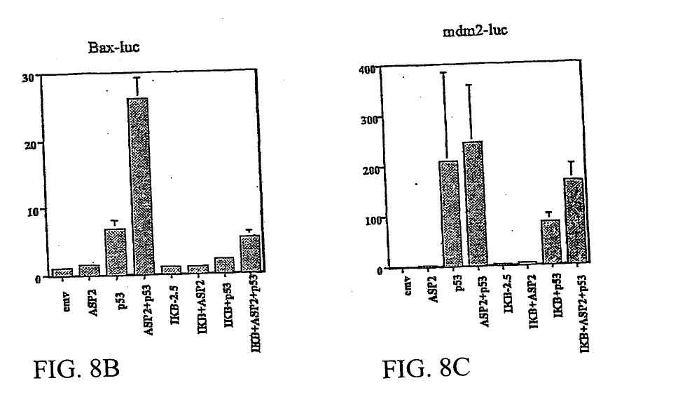

Since ASP family members are a common partner between p53 and p65, it is believed that ASP family members connect the apoptotic function of p53 and NF-kappaB. Without wishing to be bound to a particular theory, a model is proposed (FIG. 8A). p53 may induce the DNA binding activity of p65 by interacting with the nuclear iASPP and allow p65 to bind DNA. In addition, Ikb could inhibit p53-induced apoptosis by binding to p65 and releasing iASPP. The increased nuclear concentration of iASPP can then interact with p53 and prevent ASPP2 or ASPP1 to stimulate the transactivation function of p53.

To demonstrate that expression of Ikb reduces p53-induced apoptosis in the presence of ASPP1 or ASPP2, the following methods were used. Saos-2 cells were transfected with vectors encoding ASPP2, IkB, and p53 (alone or in combination), and the induction of apoptosis measured as described in Example 6. In Ikb-expressing cells, 7.2% of the cells die of apoptosis compared to 4.6% of cells transfected with in vector alone transfected cells. The effect of Ikb on p53-induced apoptosis was also minimal since the percentage of apoptotic cells detected in p53 versus p53+Ikb expressing cells were 12% and 11% respectively. This could be due to the very low level of ASPP1 and ASPP2 expression in Saos-2 cells. In agreement with the results described in the Examples above, co-expression of ASPP2 produced a significant enhancement of p53-induced apoptosis. The percentage of apoptotic cells in p53+ASP2 transfected cells was 30%. The co-expression of Ikb was able to reduce the amount of apoptotic cells induced by p53 and ASPP2 from 30% to 16%. This result indicates that Ikb reduces p53-induced apoptosis by preventing or decreasing ASPP2's abiltity to stimulate p53 function. Similar results were obtained when Ikb was co-expressed with p53 and ASPP1.

As described in the Examples above, ASPP2 enhances the apoptotic function of p53 by specifically stimulating the transactivation function of p53 on the promoters of pro-apoptotic genes such as Bax. Using similar methods, the effect of Ikb on the transactivation function of p53 on the Bax and mdm2 promoters in the presence or absence of ASP2 was determined. As shown in FIG. 8B, co-expression of ASPP2 and p53 stimulated the transactivation function of p53 by about 8-fold. Under the same conditions, the expression of Ikb did not show any detectable inhibition on the Bax promoter reporter activity, indicating that Ikb does not inhibit the transcriptional activity of Bax promoter non-specifically in Saos-2 cells. The co-expression of 50 ng of Ikb with p53 only showed a very little inhibition on the transactivation function of p53. However, when Ikb, ASPP2 and p53 were co-expressed, Ikb significantly decreased ASPP2-mediated stimulation of p53 transactivation function (FIG. 8B).

As described in Example 4, ASPP1 and ASPP2 can specifically stimulate the transactivation function of p53 on the Bax promoter but not the mdm2 promoter. Under the same conditions, the ability of Ikb to inhibit the transactivation function of p53 on the mdm2 promoter activity was determined. As shown in FIG. 8C, co-expression of ASPP2 had very little effect on the transactivation function of p53 on the mdm2 promoter. In addition, Ikb hardly decreased the transactivation function of p53 on the mdm2 promoter even in the presence of ASPP2. The results indicate that Ikb can decrease or even inhibit the apoptotic function of p53 by preventing or decreasing the ability of ASPP1 or ASPP2 to stimulate the transactivation function of p53.

To demonstrate the role of the ASP family in connecting with the p53 and the NFkb pathway, the effect of the ASPP2 and p65 relA interaction on the apoptotic function of p53 was determined. Based on the working model in FIG. 8A, the p65/ASP interaction may facilitate the nuclear entry of ASP protein, thus allowing the p53/ASP interaction and the release of nuclear iASPP to bind to the nuclear p65. Residues 176-406 of p65 bind to ASPP2 and iASPP.

As a transcription factor, p65 can transactivate many target genes. Since p53-induced apoptosis involves p65 and is correlated with the increased DNA-binding activity of NFkB, the DNA-binding activity of p65 may be needed to co-operate with p53 to induce apoptosis. The ability of ASP proteins to bind both p53 and p65 places the ASP family in a central role. ASP binding to p65 may be a mediator for the p53 induced DNA binding activity of p65. However, the co-expression of p53 failed to induce the transcriptional activity of p65 on its reporter. The co-expression of p53 and ASPP2 also failed to show any significant effect on the transactivation function of p65. This result indicates that ASP was not the messenger that delivers the signals from p53 to p65. Nevertheless, ASP may enable p65 to co-operate with p53 to induce apoptosis.

To determine whether the action of ASP needs the DNA-binding activity of p65 NFkB, the following methods were used. One hundred amino acids of p65 were removed from the N-terminus that contains the DNA binding region of p65. The Δp65 construct was transcriptionally inactive when tested on the NFkb reporter plasmid. In addition, both p65 and Δp65 stimulated the apoptotic function of p53 in the presence of ASP2, and that Δp65 was even more active than p65 in enhancing the apoptotic function of p53. This may be due to the fact that Δp65 is more nuclear than p65.

These data indicate that p65 can influence the apoptotic function of p53 independent of the DNA-binding activity of p65. Hence, the interaction of ASPP2-p65 is the proposed mechanism of action.

### EXAMPLE 12

### Bcl-2 Prevents ASPP1 and ASPP2 from Enhancing the Apoptotic Function of p53

The anti-apoptotic function of the Bcl-2 oncoprotein is known, as is the fact that p53-induced apoptosis can be inhibited by Bcl-2. Furthermore, it is known that Bcl-2 interacts with ASPP2. However, the biological consequences of this interaction are not known. Using the methods described in the above examples, the ability of Bcl-2 to inhibit p53-induced apoptosis by preventing ASPP1 and ASPP2 from stimulating p53 was determined.

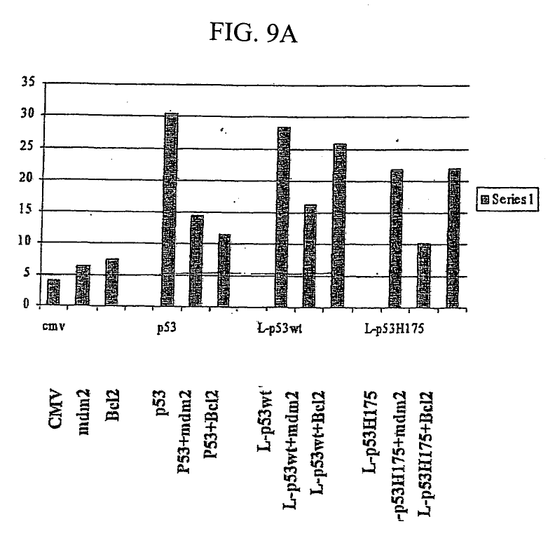

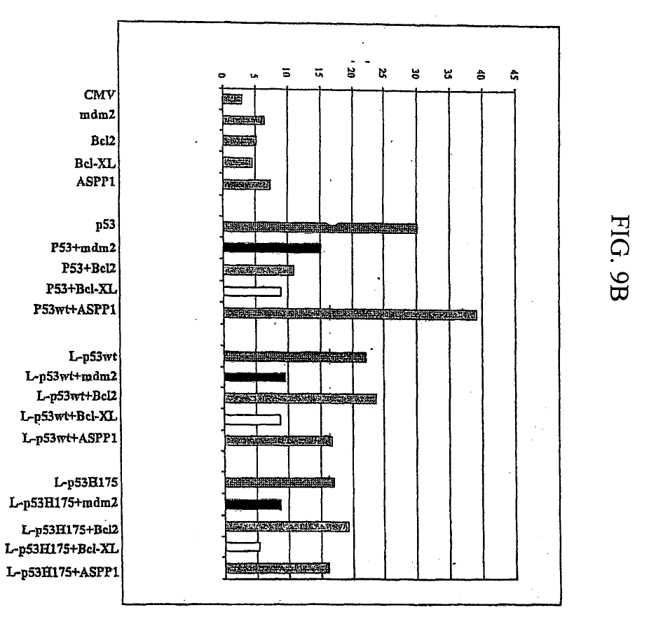

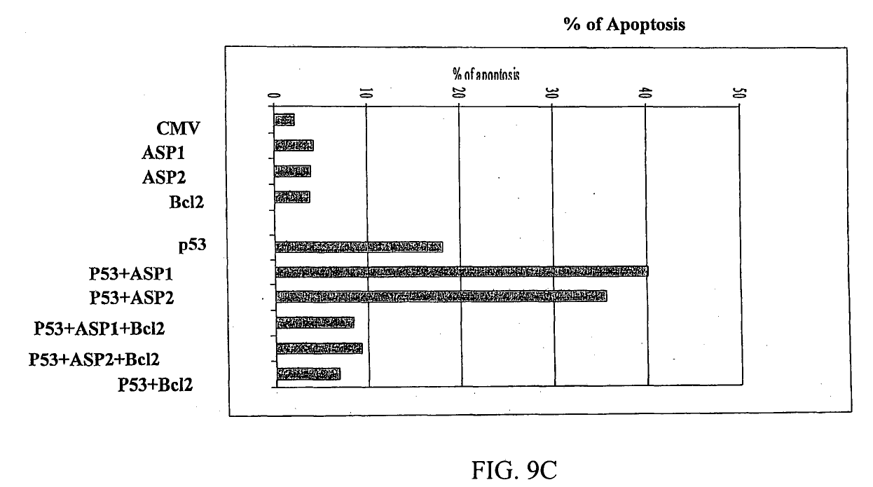

As shown in the above examples, ASPP1 and ASPP2 stimulate the apoptotic function of p53 by enhancing the DNA binding and transactivation function of p53 on promoters of apoptotic genes such as Bax and PIG3. To determine if ASP can enhance the apoptotic function of p53 independently of its transactivation function, the following methods were used. Apoptosis was induced in Saos-2 cells by the expression of wild type p53 or a transcriptionally inactive p53, p53H175-L, a mutant p53 which is targeted to mitochondria by a leader sequence and which induces apoptosis independent of the transactivation function of p53. The apoptotic function of wild type p53 was stimulated by the expression of ASPP1 and ASPP2. However, co-expression of ASPP1 and ASPP2 failed to enhance the apoptotic function of p53H175-L. Only wild type p53-induced apoptosis was decreased by co-expression of Bcl-2. Under the same conditions, co-expression of Bcl-2 failed to decrease or inhibit apoptosis induced by p53H175-L (FIG. 9A). Such selective inhibition of p53-induced apoptosis was not observed with Bcl-XL, another inhibitor of apoptosis in the Bcl-2 family (FIG. 9B).

The close association between the ability of ASP to stimulate and the ability of Bcl-2 to inhibit the apoptotic function of p53 indicates that Bcl-2 reduces or inhibits p53-induced apoptosis by preventing or decreasing ASP's ability to stimulate p53. This was confirmed by the data shown in FIG. 9C, that Bcl-2 effectively prevented ASPP1 and ASPP2 from enhancing the apoptotic function of p53.

### EXAMPLE 13

### iASPP is an Oncogene

It is demonstrated in the above examples that iASPP can inhibit p53-induced apoptosis in various cell lines and that its expression level is up-regulated in breast carcinoma cells in vivo. These data indicate that iASPP could be an oncogene. Since the tumour suppression function of p53 is linked to its ability to induce apoptosis, inhibition of p53-induced apoptosis may remove the tumour suppression function of p53.

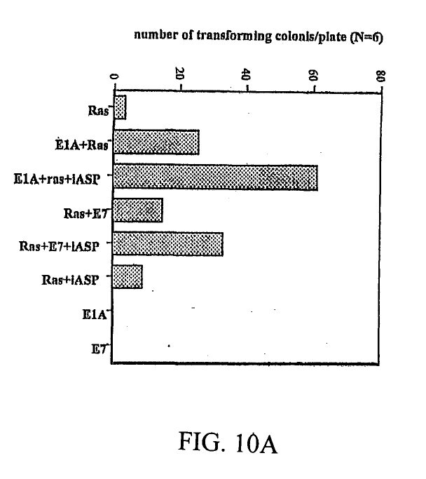

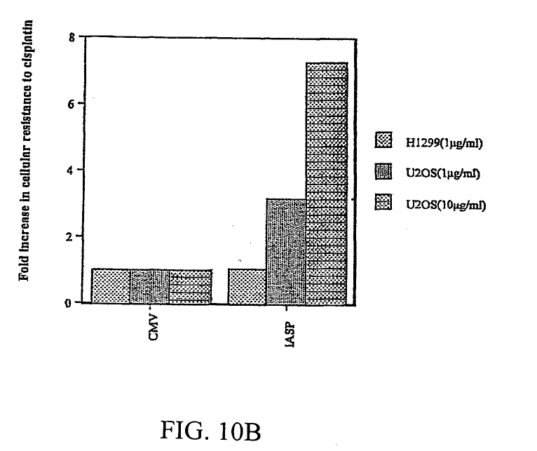

To demonstrate the oncogenic function of iASPP, rat embryo fibroblasts (REFs) were transfected with plasmids expressing iASPP and the oncoprotein, E7. The expression of iASPP enhanced the transforming function of E7 significantly (FIG. 10A). This demonstrates that iASPP is an oncogene.

Many chemotherapy drugs are DNA-damage agents and induce apoptosis via p53-dependent pathway. Therefore, the ability of iASPP to inhibit p53-induced apoptosis may make cells more resistant to the cytotoxic effect of chemotherapy drugs such as cisplatin. To demonstrate this, MCF-7 cells (a human breast cancer cell line) were transfected with an iASPP-expressing plasmid. The cellular resistance to the cytotoxic effect of cisplatin were compared between iASPP-expressing and non-expressing iASPP MCF-7 cells. Expression of iASPP enhanced the cellular resistance by about 2.5 fold (FIG. 10B). Such an increase in cellular resistance to cisplatin is significant with respect to cancer treatment.

In addition, the high level of expression of iASPP explains why wild type p53 is not functional in some human tumour cells, and can be used to predict tumour response to treatments. For example, many cytotoxic agents act via p53. However, high levels of iASPP expression results in iASPP binding to p53, which may prevent or decrease the ability of the agent to act on p53. Therefore, screening a subject to determine their level of iASPP expression or activity can be used to predict how the subject will respond to a cytotoxic agent that acts via p53. Subjects having undesired levels of iASPP could be administered an iASPP inhibitor, to increase the ability of the tumor in the subject to respond to the cytotoxic agents that act via p53. In addition, iASPP overexpressing cells can be used to identify effective chemotherapy drugs.

### EXAMPLE 14

### High levels of ASPP1 and ASPP2 Induce p53-Independent Apoptosis

As disclosed in Example 6, expression of ASPP1 or ASPP2 induced small but detectable amount of apoptosis in the p53 null cell line, Saos-2. In addition, it is known that high levels expression of ASPP2 (140 fold above endogenous ASPP2 level) cause apoptosis in 293 cells where wild type p53 was inactivated by an adenovirus protein E1B indicating that ASP can induce apoptosis independent of p53 when expressed at high level.

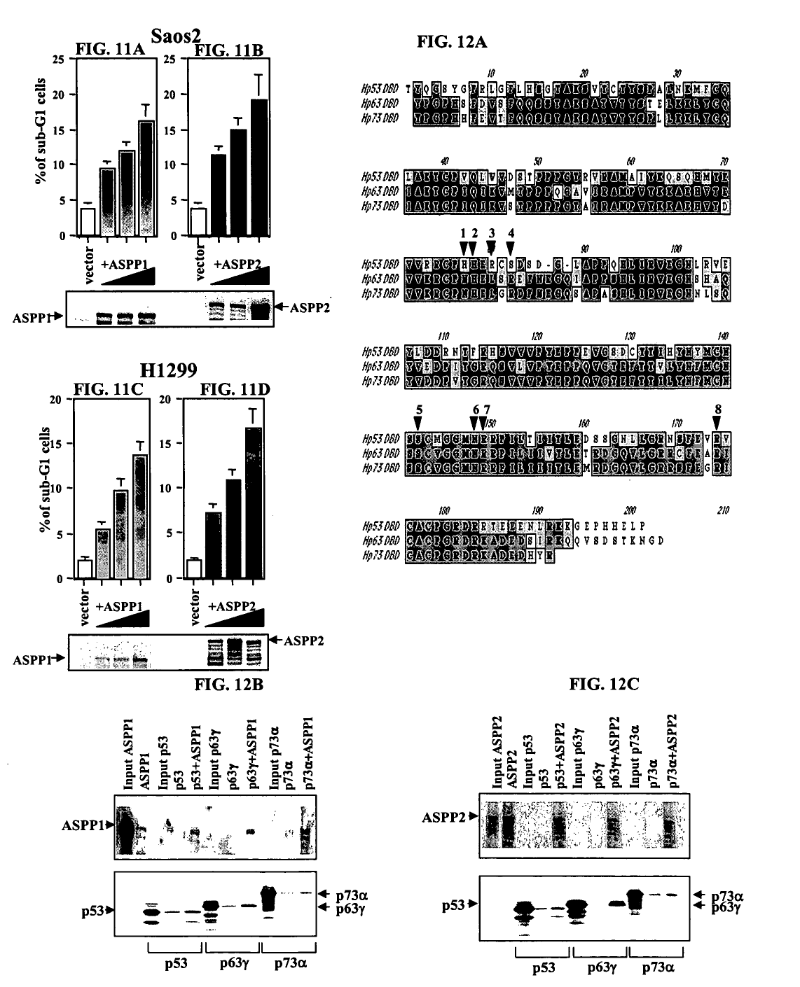

To demonstrate that ASPP1 and ASPP2 can induce apoptosis independent of p53, increasing amounts of ASPP1 or ASPP2 expressing plasmids were introduced into two p53 null cell lines, Saos-2 and H1299 as follows. Cells (10⁶) were plated 24-48 hours prior to transfection in 10 cm plates. Cells were grown in DMEM supplemented with 10% FCS and transfected with 2 μg of a plasmid expressing CD20 as a transfection marker. Increasing amounts of ASPP1 and ASPP2 were transfected (7.5 μg, 15 μg and 25 μg). 36 hours after the transfection, both attached and floating cells were harvested and analysed using flow cytometry as follows. The transfected cells were gated based on the expression of CD20. The percentage of apoptotic cells was measured by the accumulation of cells with a sub-G1 DNA content derived from at least three individual experiments. The bar graphs shown in FIGS. 11A-11D represent the percentage of apoptotic cells 36 hours after transfection.

To determine the amount of protein expression, Saos-2 cells (5×10⁵) were plated 24 hours prior to transfection in 6 cm dishes. All transactivation assays contained 1 μg of reporter plasmid and 50 ng of p53, 35 ng of p63α, 25 ng of p73γ, 4 μg of ASPP1 or AS-P2, as indicated. To determine the amount of gene activation, after transfection, the cells were lysed in Reporter Lysis Buffer 16-24 hours post-wash and assayed using the Luciferase Assay kit (Promega, Wis.). The fold activation of a particular reporter was determined by the activity of the transfected plasmid above the activity of vector alone.

In Saos-2 cells (FIGS. 11A-11B), the expression of ASPP1 or ASPP2 caused 2-3 fold increase in apoptosis while in H1299 cells (FIGS. 11C-11D) the number of apoptotic cells detected in ASPP1 or ASPP2 expressing cells was 3-7 fold higher than that of vector alone transfected cells. Hence high levels expression of ASPP1 and ASPP2 can induce apoptosis independent of p53. The amount of protein expression is shown in the lower panels of FIGS. 11A-11D.

### EXAMPLE 15

### ASPP1 and ASPP2 Interact with p63 and p73

As described above in Examples 3 and 6, ASPP1 and ASPP2 interact with the DNA binding domain of p53 and stimulate its apoptotic function. In addition, five out of eight p53 residues reported to bind the C-terminus of ASPP2 are present in p63 and p73 (FIG. 12A), indicating that ASPP1 and ASPP2 can interact with p63 and p73. To demonstrate that ASPP1 and ASPP2 also interact with p63 and p73 to influence their apoptotic function, the following methods were used.

Saos-2 and H1299 cells express the p53 family members p63 and p73, both of which induce apoptosis. The transcriptionally active isoforms of p63 and p73, p63γ and p73α, were chosen to represent each of the family members. p53, p63γ and p73α were in vitro translated and labelled with ³⁵S-methionine. V5-tagged ASPP1 and ASPP2 proteins were in vitro translated with cold methionine using the TNT T7 Quick coupled Transcription/Translation System (Promega). Cell lysates were incubated at 30° C. for 1 hour and then immunoprecipitated with anti-V5 antibody on protein G agarose beads.

The agarose beads were added to the binding reactions and incubated on a rotating wheel at 4° C. for 16 hours. The beads were then washed with PBS. The bound proteins were released in SDS gel sample buffer and analysed by 10% SDS-polyacrylamide gel electrophoresis (PAGE). The gels were wet transferred on to Protran nitrocellulose membrane and the resulting blots were first incubated with primary antibody and subsequently with the appropriate secondary HRP conjugated antibody (Dako). The blot was exposed to hyperfilm following the use of ECL substrate solution (Amersham Life Science). The presence of radiolabelled p53, p63γ or p73α complexed with ASPP1 or ASPP2 was detected using autoradiography and the amount of ASPP1 and ASPP2 immunoprecipitated were detected using anti-V5 antibody by western blot.

As shown in FIGS. 12B and 12C, p53, p63 or p73 were co-immunoprecipitated by antibodies specific to ASPP1 or ASPP2, indicating that ASP interacts with p63 and p73 in vitro. However, less p73α was in complex with ASPP1 and ASPP2 than that seen with p53 and p63γ.

The interaction between ASP and p63 or p73 was further demonstrated in vivo in H1299 and Saos-2 cells as follows. Cells were transfected with 1 μg of p63γ or p73α in the presence or absence of 10 μg of ASPP1 or ASPP2. Cell lysate (1 mg) was immunoprecipitated with an antiASPP1 or ASPP2 antibody (see Example 2 and 7). The immunoprecipitates were separated on an SDS gel and the presence of p63 or p73 on the immunoblots detected by mouse monoclonal antibodies 4A4 (Santa Cruz) and ER-15 (Neomarker) to p63 or p73, respectively. The presence of ASPP1 or ASPP2 was detected with antibodies YX.7 and DX5410, respectively.

In some methods, larger amounts of cell lysate (2 mg) were immunoprecipitated with rabbit antibodies ASPP1.88 or BP77 to ASPP1 and ASPP2, respectively. The expression of ASPP1, ASPP2, p63γ and p73α was detected as described above.

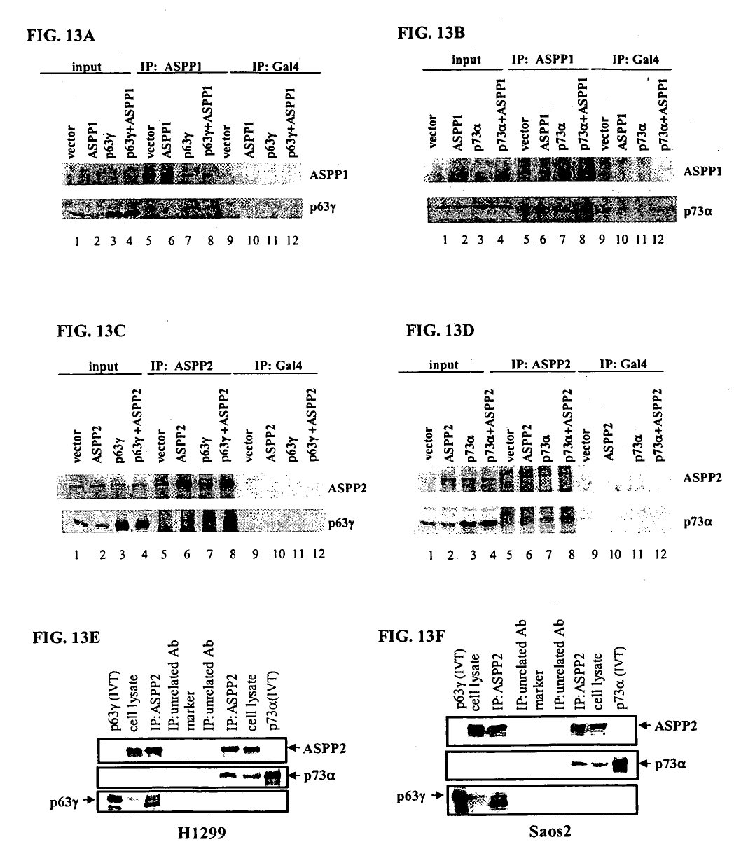

The antiASPP1 antibody immunoprecipitated endogenous and transfected ASPP1. The antiASPP1 antibody co-immunoprecipitated transfected p63γ and p73α through endogenous ASPP1 as well as the transfected ASPP1 (FIGS. 13A and 13B lanes 7,8). Similarly, the antiASPP2 antibody which immunoprecipitated endogenous and transfected ASPP2 also co-immunoprecipitated transfected p63γ and p73α (FIGS. 13C and 13D, lanes 7,8). Under the same conditions, the control antibody Gal4 failed to co-immunoprecipitate p63γ or p73α. The interaction between endogenous p63γ and transfected ASPP2 was also detected (FIG. 13C, lane 6) although no interaction was detected between p73α and transfected ASPP2 under the same conditions (FIG. 13D, lane 6). However, when large amounts of cell lysate were used the interaction between endogenous ASPP2 and p63γ or p73α was detected (FIGS. 13E-13F). The control antibody Gal4 did not immunoprecipitate either ASPP2 or p63γ or p73α proteins, indicating the interaction is specific in both cell lines.

### EXAMPLE 16

### ASPP1 and ASPP2 Stimulate the Transactivation Function of p63 and p73 on the Bax Promoter

As described in Example 4, binding of ASPP1 and ASPP2 to p53 stimulates the transactivation function of p53 on promoters of pro-apoptotic genes such as Bax and PIG3. To demonstrate that the binding of ASPP1 or ASPP2 can also increase the transactivation function of p63 and p73, the methods described in Example 4 were used.

Bax and mdm2 promoters were used to measure the transactivation function of p53, p63 and p73. The data is shown as: the activity of p53+ASPP/activity of p53 alone. The expression level of transfected proteins was detected using 40 μg of the respective lysates using the antibodies V5, DX.5410, DO1, 4A4 and p73. The luciferase reporter plasmids responsive to p53 were Bax-luc and mdm2-luc. Results were derived from at least three independent experiments.

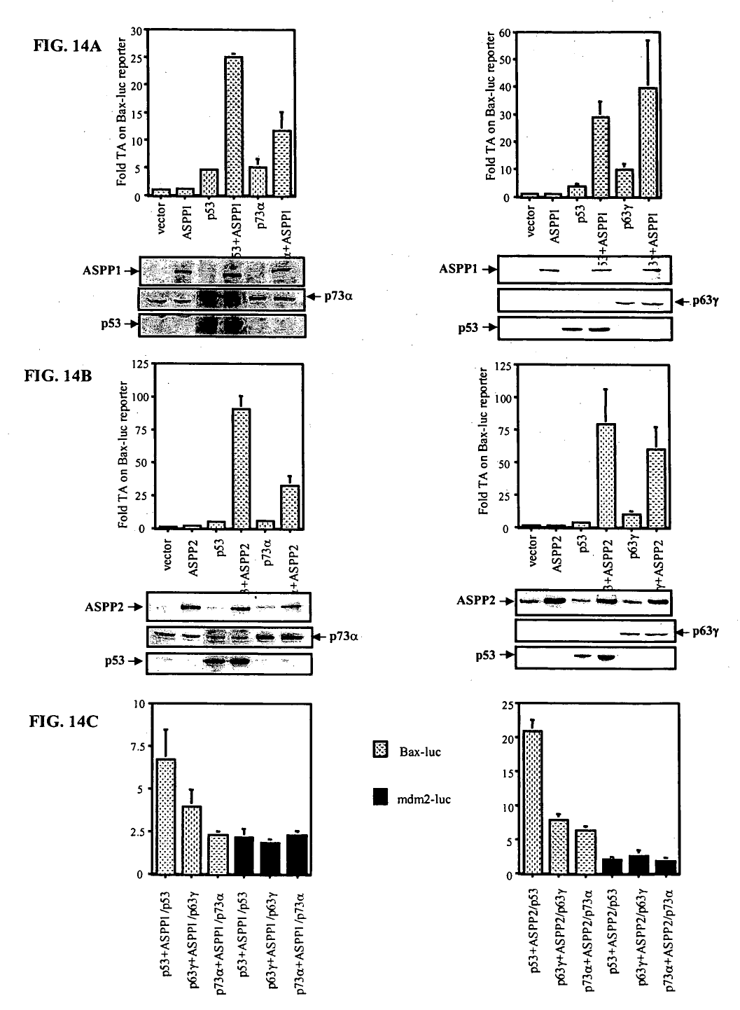

The expression of ASPP1 and ASPP2 enhanced the ability of p63 and p73 to transactivate the Bax promoter (FIGS. 14A and 14B). The expression of ASPP1 stimulated the transactivation function of p53 by around 6 fold and the transactivation function of p63γ and p73α by 4 and 3 fold, respectively. Co-expression of ASPP2 with p53 enhanced the transactivation function of p53 on the Bax promoter by 20 fold, however, it stimulated p63 and p73 only by 7 and 6 fold, respectively.

To compare the degree of activation of different p53 family members by ASPP1 and ASPP2, the luciferase counts derived from each p53 family member plus ASPP were divided by that of the p53 family member alone. This calculation showed that the ability of ASPP1 and ASPP2 to stimulate the transactivation function of p53 is greater than that seen with p63γ and p73α (FIG. 14C). Co-expression of ASPP1 and ASPP2 failed to stimulate the transactivation function of p63γ and p73α on the mdm2 promoter (FIG. 14C). This is consistent with the results described in Example 4.

### EXAMPLE 17

### ASPP1 and ASPP2 Enhance the Apoptotic Function of p63 and p73

To demonstrate that ASPP1 and ASPP2 stimulate the apoptotic function of p63 and p73, in addition to their ability to stimulate the transactivation function of p63 and p73 on promoters of a pro-apoptotic gene such as Bax, the following methods were used. Saos-2 cells were transfected with 1 μg of human p53, or 1 μg or 2.5 μg of p63γ or p73α, and 10 μg of ASPP1 and ASPP2 as indicated. The transfected cells were analyzed by flow cytometry as described in Example 6.

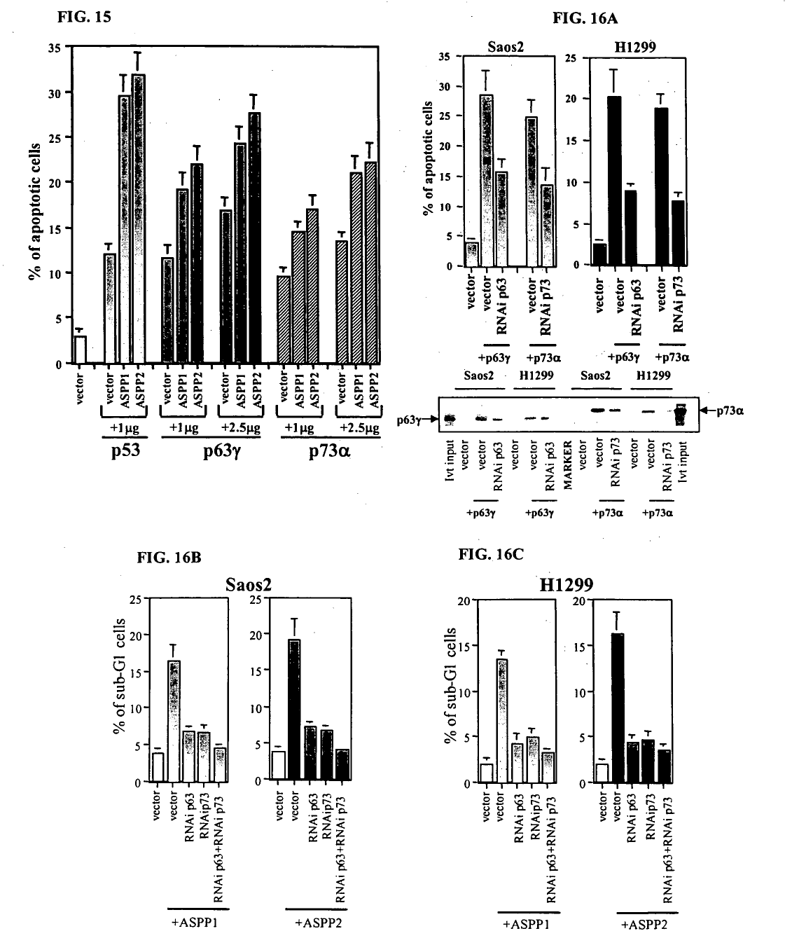

Co-expression of ASPP1 and ASPP2 enhanced the apoptotic function of p63γ and p73α (FIG. 15). The extent of increase in the apoptotic function of p63γ and p73α is lower than that seen with p53. This is in agreement with the results shown in FIG. 14, where ASPP1 and ASPP2 stimulate the transactivation function of p53 better than p63γ and p73α.

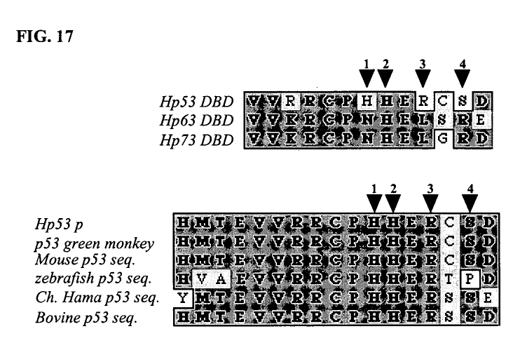

It is possible that ASPP1 and ASPP2 have a slightly larger impact on the activity of p53 than that of p63γ and p73α, because although the DNA binding domains of p53, p63 and p73 are highly homologous, three ASPP contact residues that are conserved in p53 throughout evolution are not conserved in p63 and p73 (FIG. 17). It is possible that these residues are important for an efficient co-operation with ASPP1 and ASPP2. However, significant differences in in vitro binding between ASPP1 and ASPP2 and the p53 family members were not observed, although functional differences between the family members in vivo were observed.

### EXAMPLE 18

### The p53 Independent Apoptotic Function of ASPP1 and ASPP2 is Mediated by p63 and p73

To demonstrate that the p53-independent apoptotic function of ASPP is mediated by p63 and p73, RNA interference was used to decrease or inhibit the activity of endogenous p63 and p73 in Saos2 and H1299 cells.

Saos-2 or H1299 cells were transfected with the expression plasmid of a cell surface marker CD20 (2 μg) together with 1 μg p53, 1 or 2.5 μg of p63γ or p⁷³α, in the presence or absence of 25 μg of ASPP1 or ASPP2, and 10 μg of pSuper plasmids containing p63 RNAi or p73 RNAi as indicated. RNAi oligonucleotides (19 bp) were ligated into pSuper expression plasmids as described previously (Brummelkamp et al., Science 296:550-3, 2002). The sequences of p63 and p73 sense and antisense oligonucleotides used were (lowercase indicates the vector sequence from pSuper; uppercase indicates the target sequence of the RNAi): for p63,

| | |
|---|---|
| 5′gatccccTGAATTCCTCAGTCCAGAGGttcaagagaCCTCTGGACTGAGGAATTCAtttttggaaa | (sense; SEQ ID NO: 7) |
| and | |
| 5′agcttttccaaaaaTGAATTCCTCAGTCCAGAGGtctcttgaaCCTCTGGACTGAGGAATTCAggg; | (antisense; SEQ ID NO: 8) |
| for p73, | |
| 5′gatccccGCCGGGGGAATAATGAGGTttcaagagaACCTCATTATTCCCCCGGCttttggaaa | (sense; SEQ ID NO: 9) |
| and | |
| 5′agcttttccaaaaaGCCGGGGGAATAATGAGGTtctcttgaaACCTCATTATTCCCCCGGCggg. | (antisense; SEQ ID NO: 10) |

Co-expression of p63 and p73 RNAi specifically inhibited the apoptosis induced by p63 and p73, respectively, in both Saos-2 and H1299 cells (FIG. 16A) and reduced p63 and p73 protein expression (FIG. 16B). Co-transfection of p63 or p73 RNAi to reduce the expression of endogenous p63 or p73 significantly reduced the apoptotic function of ASPP1 and ASPP2 in both Saos-2 and H1299 cells demonstrating that in the absence of p53, ASPP1 and ASPP2 induce apoptosis via endogenous p63 and p73 (FIGS. 16C and 16D). When p63 and p73 RNAi were co-expressed together, almost 80% of the apoptotic function of ASPP1 and ASPP2 was inhibited. These findings demonstrate that ASPP1 and ASPP2 are common activators of all p53 family members and most of the p53 independent apoptotic function of ASPP1 and ASPP2 is mediated by p63 and p73.

### EXAMPLE 19

### Methods of Treating a Tumor

This example describes methods that can be used to treat a tumor, such as a tumor in a subject. Examples of tumors that can be treated using the disclosed therapeutic agents include, but are not limited to, p53 expressing tumors, tumors that express mutant p53 and p63 or p73, and tumors that do not express functional p53 but express functional p63 or p73. Particular tumors include tumors of the lung and breast.

In particular examples, the expression profile of the tumor is determined prior to administering a therapeutically effective amount of the ASPP agent, to determine if the tumor would respond to the therapies disclosed herein. For example, a sample of the tumor is obtained from the subject, and the amount of p53 (wild-type or mutant), p63, and p73 expression determined. Expression of these molecules can be determined using standard molecular biology techniques, such as western blotting, Southern blotting, and real-time RT-PCR. In some examples, the amount of functional expression, such as an amount of functional p53 protein expression, is determined, for example by determining an amount of p53 activity.

Subjects having a tumor that expresses p63 or p73 (alone or in the presence of p53, mutant p53, or no detectable functional p53), or a tumor that expresses p53 but not p63 or p73, can be administered a therapeutically effective amount of an agent that increases ASPP1 or ASPP2 activity, for example by administration of an ASPP1 or ASPP2 protein, nucleic acid molecule, agonist, or mimetic thereof. In one particular example, the tumor expresses increased amounts of p63 or p73, as compared to a level of expression in a non-tumor cell of the same cell type (such as a normal epithelial cell). Such agents can be administered systemically or directly to the tumor, or by any other appropriate route. In addition, one or more additional anti-tumor agents (in a therapeutically effective amount), in combination with an agent that increases ASPP1 or ASPP2 activity, can be administered to the subject having a tumor. Such anti-tumor agents can be administered at the same time as the agent that increases ASPP1 or ASPP2 activity, or at some other time, such as before or after administration of the agent that increases ASPP1 or ASPP2 activity. The disclosed therapeutic compositions can be administered once or repeatedly (such as daily, weekly, or monthly) as needed.

Similar methods of increasing ASPP1 or ASPP2 activity can be used to treat a condition mediated by decreased p63 or p73 activity. Examples of such conditions include, but are not limited to, defects in ectodermal development, such as ectrodactyly, ectodermal dysplasia and facial Clefts (EEC).

### EXAMPLE 20

### Disruption of Gene Expression

This example describes methods that can be used to disrupt expression of an iASPP gene and thereby decrease activity of iASPP proteins, and thereby increase apoptosis. Such methods are useful when it is desired to decrease a tumor. In a particular example, disrupted expression of SEQ ID NO: 5 (or variants thereof having similar activity) in a host cell is used to treat a subject having a tumor.

Similar methods can be used to disrupt expression of an ASPP1 or ASPP2 gene and thereby decrease activity of ASPP proteins, and thereby decrease apoptosis. In one example, such methods are useful when it is desired to decrease cell death. In a particular example, disrupted expression of SEQ ID NOS: 1 or 3 (or variants thereof having similar activity) in a cell is used to treat a subject having heart disease or brain disease (such as Alzheimer's). In another example, such methods are useful when decreased p73 or p63 activity is desired, for example in the treatment of disorders associated with increased p63 or p73 activity, such as neuroblastoma, colorectral cancer, breast cancer, hepatocellular carcinoma, and liver cholangiocarcinoma.

Methods useful for disrupting gene function or expression are the use of antisense oligonucleotides, siRNA molecules, RNAi molecules, ribozymes, and triple helix molecules. Techniques for the production and use of such molecules are well known to those of skill in the art. The molecules disclosed in this example can be administered as part of a pharmaceutical composition. In one example, the composition is sterile and includes a therapeutically effective amount of molecule in a unit of weight or volume suitable for administration to a subject.

Antisense Methods

To design antisense oligonucleotides, a host mRNA sequence is examined. Regions of the sequence containing multiple repeats, such as TTTTTTTT, are not as desirable because they will lack specificity. Several different regions can be chosen. Of those, oligos are selected by the following characteristics: those having the best conformation in solution; those optimized for hybridization characteristics; and those having less potential to form secondary structures. Antisense molecules having a propensity to generate secondary structures are less desirable.

Plasmids including antisense sequences that recognize one or more of SEQ ID NOS: 1, 3 and 5 can be generated using standard methods. For example, cDNA fragments or variants coding for an ASP or iASPP protein are PCR amplified. The nucleotides are anplified using Pfu DNA polymerase (Stratagene) and cloned in antisense orientation a vector, such as pcDNA vectors (InVitrogen, Carlsbad, Calif.). The nucleotide sequence and orientation of the insert can be confirmed by sequencing using a Sequenase kit (Amersham Pharmacia Biotech).

Generally, the term “antisense” refers to a nucleic acid capable of hybridizing to a portion of an RNA sequence (such as mRNA) by virtue of some sequence complementarity. The antisense nucleic acids disclosed herein can be oligonucleotides that are double-stranded or single-stranded, RNA or DNA or a modification or derivative thereof, which can be directly administered to a cell, or which can be produced intracellularly by transcription of exogenous, introduced sequences.

Antisense nucleic acids are polynucleotides, and can be oligonucleotides (ranging from about 6 to about 100 oligonucleotides). In one example, an antisense polynucleotide recognizes one or more of SEQ ID NOS: 1, 3 and 5, such as at least 10, or at least 15 contiguous nucleotides of SEQ ID NOS: 1, 3, or 5. In specific examples, the oligonucleotide is at least 10, 15, or 100 nucleotides, or a polynucleotide of at least 200 nucleotides. However, antisense nucleic acids can be much longer. The nucleotide can be modified at the base moiety, sugar moiety, or phosphate backbone, and can include other appending groups such as peptides, or agents facilitating transport across the cell membrane (Letsinger et al., Proc. Natl. Acad. Sci. USA 1989, 86:6553-6; Lemaitre et al., Proc. Natl. Acad. Sci. USA 1987, 84:648-52; WO 88/09810) or blood-brain barrier (WO 89/10134), hybridization triggered cleavage agents (Krol et al., BioTechniques 1988, 6:958-76) or intercalating agents (Zon, Pharm. Res. 5:539-49, 1988).

An antisense polynucleotide (including oligonucleotides) that recognizes one or more of SEQ ID NOS: 1, 3 or 5, can be modified at any position on its structure with substituents generally known in the art. For example, a modified base moiety can be 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl)uracil, and 2,6-diaminopurine.

An antisense polynucleotide that recognizes one or more of SEQ ID NOS: 1, 3 or 5, can include at least one modified sugar moiety such as arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

In a particular example, an antisense polynucleotide that recognizes one or more of SEQ ID NOS: 1, 3, or 5 is an α-anomeric oligonucleotide. An α-anomeric oligonucleotide forms specific double-stranded hybrids with complementary RNA in which, contrary to the usual β-units, the strands run parallel to each other (Gautier et al., Nucl. Acids Res. 15:6625-41, 1987). The oligonucleotide can be conjugated to another molecule, such as a peptide, hybridization triggered cross-linking agent, transport agent, or hybridization-triggered cleavage agent. Oligonucleotides can include a targeting moiety that enhances uptake of the molecule by host cells. The targeting moiety can be a specific binding molecule, such as an antibody or fragment thereof that recognizes a molecule present on the surface of the cell.

Polynucleotides disclosed herein can be synthesized by standard methods, for example by use of an automated DNA synthesizer. As examples, phosphorothioate oligos can be synthesized by the method of Stein et al. (Nucl. Acids Res. 1998, 16:3209), methylphosphonate oligos can be prepared by use of controlled pore glass polymer supports (Sarin et al., Proc. Natl. Acad. Sci. USA 85:7448-51, 1988). In a specific example, antisense oligonucleotide that recognizes one or more of SEQ ID NOS: 1, 3 or 5 includes catalytic RNA, or a ribozyme (see WO 90/11364, Sarver et al., Science 247:1222-5, 1990). In another example, the oligonucleotide is a 2′-0-methylribonucleotide (Inoue et al., Nucl. Acids Res. 15:6131-48, 1987), or a chimeric RNA-DNA analogue (Inoue et al., FEBS Lett. 215:327-30, 1987).

The antisense polynucleic acids disclosed herein include a sequence complementary to at least a portion of an RNA transcript of a gene, such as SEQ ID NOS: 1, 3 or 5. However, absolute complementarity, although advantageous, is not required. A sequence can be complementary to at least a portion of an RNA, meaning a sequence having sufficient complementarily to be able to hybridize with the RNA, forming a stable duplex; in the case of double-stranded antisense nucleic acids, a single strand of the duplex DNA may thus be tested, or triplex formation can be assayed. The ability to hybridize depends on the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The relative ability of polynucleotides (such as oligonucleotides) to bind to complementary strands is compared by determining the Tₘ of a hybridization complex of the poly/oligonucleotide and its complementary strand. The higher the Tₘ the greater the strength of the binding of the hybridized strands. As close to optimal fidelity of base pairing as possible achieves optimal hybridization of a poly/oligonucleotide to its target RNA.

The amount of antisense nucleic acid that is effective in the treatment of a particular disease or condition (the therapeutically effective amount) depends on the nature of the disease or condition, and can be determined by standard clinical techniques. For example, it can be useful to use compositions to achieve sustained release of an antisense nucleic acid, for example an antisense molecule that recognizes one or more of SEQ ID NOS: 1, 3, or 5. In another example, it may be desirable to utilize liposomes targeted via antibodies to specific cells.

Ribozymes

As an alternative to antisense inhibitors, catalytic nucleic acid compounds, such as ribozymes or anti-sense conjugates, can be used to inhibit gene expression. Ribozymes can be synthesized and administered to the subject, or can be encoded on an expression vector, from which the ribozyme is synthesized in the targeted cell (as in WO 9523225, and Beigelman et al. Nucl. Acids Res. 1995, 23:4434-42). Examples of oligonucleotides with catalytic activity are described in WO 9506764. Conjugates of antisense with a metal complex, such as terpyridylCu (II), capable of mediating mRNA hydrolysis, are described in Bashkin et al. (Appl. Biochem Biotechnol. 54:43-56, 1995).

Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Methods of using ribozymes to decrease or inhibit RNA expression are known in the art. An overview of ribozymes and methods of their use is provided in Kashani-Sabet (J. Imvestig. Dermatol. Symp. Proc., 7:76-78, 2002).

Ribozyme molecules include one or more sequences complementary to the target host mRNA and include the well-known catalytic sequence responsible for mRNA cleavage (see U.S. Pat. No. 5,093,256, herein incorporated by reference).

A ribozyme gene directed against any of SEQ ID NOS: 1, 3, or 5 can be delivered to a subject endogenously (where the ribozyme coding gene is transcribed intracellularly) or exogenously (where the ribozymes are introduced into a cell, for example by transfection). Methods describing endogenous and exogenous delivery are provided in Marschall et al. (Cell Mol. Neurobiol. 14:523-38, 1994).

Specific ribozyme cleavage sites within any potential RNA target are initially identified by scanning the target molecule for ribozyme cleavage sites that include the following sequence: GUA, GUU and GUC. Once identified, short RNA sequences of between 15 and ribonucleotides corresponding to the region of the target gene containing the cleavage site can be evaluated for predicted structural features, such as secondary structure, that may render the oligonucleotide sequence unsuitable. The suitability of candidate targets can also be evaluated by testing their accessibility to hybridization with complementary oligonucleotides, using ribonuclease protection assays.

For example, a plasmid that contains a riboyzme gene directed against iASPP placed behind a promoter, can be transfected into the cells of a subject, for example a subject having a tumor. Expression of this plasmid in a cell will decrease or inhibit iASPP RNA expression in the cell. Other examples of using ribozymes to decrease or inhibit RNA expression can be found in WO 01/83754 (herein incorporated by reference).

Triple Helix Molecules

Nucleic acid molecules used in triplex helix formation should be single stranded and composed of deoxynucleotides. The base composition of these oligonucleotides is ideally designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of guanidine residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with one strand of a duplex first and then the other, eliminating the necessity for a sizeable stretch of either purines or pyrimidines to be present on one strand of a duplex.

### EXAMPLE 21

### Pharmaceutical Compositions and Modes of Administration

Disclosed are compositions that include ASPP1 or ASPP2 proteins or nucleic acid molecules, as well as ASPP1 or ASPP2 mimetics or agonists. Also disclosed are compositions that include inhibitors of iASPP, such as an iASPP antagonists. Such compositions can be used to treat a disorder associated with a defect in apoptosis, such as a tumor.

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acid molecules as part of a retroviral or other vector. Methods of introduction include, but are not limited to, topical, intradermal, intramuscular, intraperitoneal, intravenous, intratumor, subcutaneous, intranasal, and oral routes. The compounds can be administered by any convenient route, for example by infusion or bolus injection, by absorption through epithelial or mucocutaneous linings (for example oral mucosa, rectal, vaginal and intestinal mucosa, etc.) and can be administered together with other biologically active agents. Administration can be systemic or local. In one example, pharmaceutical compositions disclosed herein are delivered locally to the area in need of treatment, for example by administration directly to a tumor, such as by injecting the tumor with the therapeutic agent.

Liposomes can be used as a delivery vehicle. Liposomes fuse with the target site and deliver the contents of the lumen intracellularly. The liposomes are maintained in contact with the target cells for a sufficient time for fusion to occur, using various means to maintain contact, such as isolation and binding agents. Liposomes can be prepared with purified proteins or peptides that mediate fusion of membranes, such as Sendai virus or influenza virus. The lipids may be any useful combination of known liposome forming lipids, including cationic lipids, such as phosphatidylcholine. Other potential lipids include neutral lipids, such as cholesterol, phosphatidyl serine, phosphatidyl glycerol, and the like. For preparing the liposomes, the procedure described by Kato et al. (J. Biol. Chem. 1991, 266:3361) can be used.

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the DNA, RNA, proteins, and specific-binding agents herein disclosed. In general, the nature of the carrier will depend on the mode of administration being employed. For instance, parenteral formulations usually include injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition can be a liquid solution, suspension, emulsion, tablet, pill, capsule, sustained release formulation, or powder. For solid compositions (such as a powder, pill, tablet, or capsule forms), conventional non-toxic solid carriers can include, for example, pharmaceutical grades of mannitol, lactose, starch, sodium saccharine, cellulose, magnesium carbonate, or magnesium stearate. The composition can be formulated as a suppository, with traditional binders and carriers such as triglycerides.

The present disclosure also provides pharmaceutical compositions that include a therapeutically effective amount of an ASPP1 or ASPP2 protein, nucleic acid molecule, mimetic, or agonist, (or an inhibitor of iASPP) alone or with a pharmaceutically acceptable carrier. The amount of ASP agent or iASPP inhibitor effective in the treatment of a particular disorder or condition can depend on the nature of the disorder or condition, and can be determined by standard clinical techniques. In addition, in vitro assays can be employed to identify optimal dosage ranges. The precise dose to be employed in the formulation can also depend on the route of administration, and the seriousness of the disease or disorder, and should be decided according to the judgment of the practitioner and each subject's circumstances. Effective doses can be extrapolated from dose-response curves derived from in vitro or animal model test systems. Furthermore, the pharmaceutical compositions or methods of treatment can be administered in combination with other therapeutic treatments, such as other agents that reduce tumor growth or metastasis.

In some examples, the desired response can be measured by determining whether signal transduction was enhanced or inhibited by the ASP or inhibitor of iASPP composition via a reporter system as described herein, by measuring downstream effects such as gene expression, or by measuring the physiological effects of the composition, such as regression of a tumor, decrease of disease symptoms, modulation of apoptosis.

In an example in which an ASPP1 or ASPP2 nucleic acid molecule, or a nucleic acid molecule that reduces iASPP activity (such as an antisense molecule) is employed to allow expression of the nucleic acid in a cell, the nucleic acid can be delivered intracellularly (for example by expression from a nucleic acid vector or by receptor-mediated mechanisms) or by an appropriate nucleic acid expression vector which is administered so that it becomes intracellular, for example by use of a retroviral vector (see U.S. Pat. No. 4,980,286), or by direct injection, or by use of microparticle bombardment (such as a gene gun; Biolistic, Dupont), or coating with lipids or cell-surface receptors or transfecting agents, or by administering it in linkage to a homeobox-like peptide which is known to enter the nucleus (for example Joliot et al., Proc. Natl. Acad. Sci. USA 1991, 88:1864-8). Alternatively, the nucleic acid molecule can be introduced intracellularly and incorporated within host cell DNA for expression, by homologous recombination.

The vector pcDNA is an example of a method of introducing the foreign cDNA into a cell under the control of a strong viral promoter (CMV) to drive the expression. However, other vectors can be used. Other retroviral vectors (such as pRETRO-ON, Clontech), also use this promoter but have the advantages of entering cells without any transfection aid, integrating into the genome of target cells only when the target cell is dividing and they are regulated. It is also possible to turn on the expression of an ASPP1 or ASPP2 nucleic acid molecule by administering tetracycline when these plasmids are used. Hence these plasmids can be allowed to transfect the cells, then administer a course of tetracycline with a course of chemotherapy to achieve better cytotoxicity. The present disclosure includes all forms of nucleic acid molecule delivery, including synthetic oligos, naked DNA, plasmid and viral, integrated into the genome or not. In particular examples, intravenous administration is used when administering a nucleic acid molecule.

In some examples, the nucleic acid molecule is targeted to particular cells. For example, a vehicle used for delivering a nucleic acid of the invention into a cell (such as a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Such proteins include capsid proteins or fragments thereof for a particular cell type, antibodies for proteins that undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

In an example where the therapeutic molecule is a specific-binding agent, such as an antibody that recognizes an ASPP1, ASPP2, or iASPP protein, administration can be achieved by direct topical administration or injection, or by use of microparticle bombardment, or coating with lipids or cell-surface receptors or transfecting agents. Similar methods can be used to administer an ASPP1, ASPP2, or iASPP protein. In a particular example, the therapeutic agent is administered with a pulmonary aerosol. Techniques for preparing aerosol delivery systems containing antibodies are well known to those of skill in the art. Ideally, such systems utilize components which will not significantly impair the biological properties of the therapeutic agents, such as the binding capacity (see, for example, Sciarra and Cutie, “Aerosols,” in Remington's Pharmaceutical Sciences, 18th edition, 1990, PP1694-1712; incorporated by reference).

The disclosure also provides a pharmaceutical pack or kit comprising one or more containers filled with one or more of the ingredients of the disclosed pharmaceutical compositions. In certain examples, other agents that increase apoptosis or otherwise favourably affect the ASPP1, ASPP2 or inhibitor iASPP compositions are included in the same kit, such as chemotherapeutic agents. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

The disclosure provides compositions of ASPP1 or ASPP2 peptides, for example a composition that includes at least 50%, for example at least 90%, of a peptide or variant, fragment, or fusion thereof. Such compositions are useful as therapeutic agents when constituted as pharmaceutical compositions with the appropriate carriers or diluents.

### EXAMPLE 22

### In Vitro Screening Assay for Agents that Modulate Apoptosis

This example describes in vitro methods that can be used to screen test agents for their ability to modulate binding of ASPP1 or ASPP2 to p53, p63, or p73. Agents that increase binding of ASPP1 or ASPP2 to p53, p63, or p73 are candidate agents for increasing apoptosis or increasing Bax promoter activity, while agents that decrease binding of ASPP1 or ASPP2 to p53, p63, or p73 are candidate agents for decreasing apoptosis, or decreasing Bax promoter activity. As disclosed in the Examples above, ASP agents increase apoptosis associated with p53, p63, and p73, while iASPP agents decrease apoptosis associated with p53 in the presence of ASPP1 or ASPP2. Therefore, screening assays can be used to identify and analyze agents that decrease or increase with this interaction. However, the present disclosure is not limited to the particular methods disclosed herein.

Agents identified via the disclosed assays can be useful, for example, in decreasing or even inhibiting apoptosis by more than an amount of apoptosis in the absence of the agent, such as a decrease of at least about 10%, at least about 20%, at least about 50%, or even at least about 90%. This decrease in apoptosis can serve to ameliorate symptoms associated with uncontrolled apoptosis, such as heart disease. Assays for testing the effectiveness of the identified agents, are discussed below.

In addition, agents identified via the disclosed assays can be useful, for example, in increasing apoptosis by more than an amount of apoptosis in the absence of the agent, such as a increase of at least about 10%, at least about 20%, at least about 50%, or even at least about 90%. This increase in apoptosis can serve to ameliorate symptoms associated with uncontrolled cell growth, such as a tumor. Assays for testing the effectiveness of the identified agents, are discussed below.

Exemplary test agents include, but are not limited to, any peptide or non-peptide composition in a purified or non-purified form, such as peptides made of D-and/or L-configuration amino acids (in, for example, the form of random peptide libraries; see Lam et al., Nature 354:82-4, 1991), phosphopeptides (such as in the form of random or partially degenerate, directed phosphopeptide libraries; see, for example, Songyang et al., Cell 72:767-78, 1993), antibodies, and small or large organic or inorganic molecules. A test agent can also include a complex mixture or “cocktail” of molecules.

The basic principle of the assay systems used to identify agents that interfere with the interaction between ASPP1 or ASPP2 and p53, p63, or p73, involves preparing a reaction mixture containing the ASP protein and a p53, p63 or p73 protein under conditions and for a time sufficient to allow the two proteins to interact and bind, thus forming a complex. In order to test an agent for inhibitory or stimulatory activity, the reaction is conducted in the presence and absence of the test agent. The test agent can be initially included in the reaction mixture, or added at a time subsequent to the addition of an ASP protein and a p53, p63 or p73 protein. Controls are incubated without the test agent or with a placebo. Exemplary controls include agents known not to bind to ASP proteins or p53, p63 or p73 proteins. The formation of any complexes between the ASP protein and the p53, p63 or p73protein is then detected.

The formation of a complex in the control reaction (where the agent in the control reaction is known to bind to ASP), but not in the reaction mixture containing the test agent, indicates that the agent interferes with the interaction of the ASP protein and the p53, p63 or p73 protein, and is therefore possibly an agent that can be used to decrease apoptosis. In contrast, formation of a complex in the reaction mixture containing the test agent, but not in the control reaction (where the agent in the control reaction is known to be unable to bind to ASP), indicates that the agent increases or stabilizes the interaction of the ASP protein and the p53, p63 or p73 protein, and is therefore possibly an agent that can be used to increase apoptosis. Similarly, greater formation of complexes in the reaction mixture containing the test agent (or stronger complex formation), than in the control reaction (where the agent in the control reaction is a wild-type p53, p63 or p73), indicates that the agent increases or stabilizes the interaction of the ASP protein and the p53, p63 or p73 protein, and is therefore possibly an agent that can be used to increase apoptosis.

The assay for agents that modulate the interaction of ASP and p53, p63 or p73 proteins can be conducted in a heterogeneous or homogeneous format. Heterogeneous assays involve anchoring the ASP protein or the p53, p63 or p73 protein onto a solid phase and detecting complexes anchored on the solid phase at the end of the reaction. In some examples, the method further involves quantitating the amount of complex formation or inhibition. Exemplary methods that can be used to detect the presence of complexes, when one of the proteins is labeled, include ELISA, spectrophotometry, flow cytometry, and microscopy. In homogeneous assays, the entire reaction is performed in a liquid phase. In either method, the order of addition of reactants can be varied to obtain different information about the agents being tested. For example, test agents that interfere with the interaction between the proteins, such as by competition, can be identified by conducting the reaction in the presence of the test agent, for example by adding the test agent to the reaction mixture prior to or simultaneously with the ASP protein and p53, p63 or p73 protein. On the other hand, test agents that disrupt or stabilize preformed complexes, such as agents with higher binding constants that displace one of the proteins from the complex, can be tested by adding the test agent to the reaction mixture after complexes have been formed. The various formats are described briefly below.

Once identified, test agents found to modulate the interaction between an ASP protein and a p53, p63 or p73 protein can be formulated in therapeutic products in pharmaceutically acceptable formulations, and used for specific treatment or prevention of a disease, such a disease associated with needed apoptosis (such as in the case of a tumor) or a disease associated with undesired apoptosis (such as heart disease).

Heterogeneous Assay System

In a heterogeneous assay system, one binding partner, either the ASP protein (SEQ ID NOS: 2 or 4) or the p53, p63 or p73 protein is anchored onto a solid surface (such as a microtiter plate), and its binding partner, which is not anchored, is labeled, either directly or indirectly. Exemplary labels include, but are not limited to, enzymes, fluorophores, ligands, and radioactive isotopes. The anchored protein can be immobilized by non-covalent or covalent attachments. Non-covalent attachment can be accomplished simply by coating the solid surface with a solution of the protein and drying. Alternatively, an immobilized antibody (such as a monoclonal antibody) specific for the protein can be used to anchor the protein to the solid surface. The surfaces can be prepared in advance and stored.

To conduct the assay, the binding partner of the immobilized species is added to the coated surface with or without the test agent. After the reaction is complete, unreacted components are removed (such as by washing) and any complexes formed will remain immobilized on the solid surface. The detection of complexes anchored on the solid surface can be accomplished in a number of ways. Where the binding partner was pre-labeled, the detection of label immobilized on the surface indicates that complexes were formed. Where the binding partner is not pre-labeled, an indirect label can be used to detect complexes anchored on the surface; for example by using a labeled antibody specific for the binding partner (the antibody, in turn, may be directly labeled or indirectly labeled with a labeled anti-Ig antibody). Depending upon the order of addition of reaction components, test compounds which decrease or increase complex formation or which disrupt or stabilize preformed complexes can be detected.

Alternatively, the reaction can be conducted in a liquid phase in the presence or absence of the test agent, the reaction products separated from unreacted components, and complexes detected; for example by using an immobilized antibody specific for one binding partner to anchor any complexes formed in solution, and a labeled antibody specific for the other binding partner to detect anchored complexes. Again, depending upon the order of addition of reactants to the liquid phase, test agents which decrease or increase complex formation or which disrupt or stabilize preformed complexes can be identified.

Homogenous Assays

In an alternate example, a homogeneous assay can be used. In this method, a preformed complex of the ASP protein and the p53, p63 or p73 protein is prepared in which one of the proteins is labeled, but the signal generated by the label is quenched due to complex formation (for example, see U.S. Pat. No. 4,109,496 by Rubenstein that utilizes this approach for immunoassays). The addition of a test substance that competes with and displaces one of the binding partners from the preformed complex will result in the generation of a signal above background. In this way, test agents that disrupt ASP protein-p53, p63 or p73 protein interactions are identified.

In contrast, the addition of a test agent that stabilizes the binding partners in the preformed complex will not increase the signal above background. In this way, test agents that stabilize ASP protein-p53, p63 or p73 protein interactions are identified.

Immobilization of Proteins

In a particular example, an ASP protein can be prepared for immobilization using recombinant DNA techniques. For example, a functional fragment (or full length) ASPP1 or ASPP2 can be fused to a glutathione-S-transferase (GST) gene using the fusion vector pGEX-5X-1, in such a manner that its binding activity is maintained in the resulting fusion protein. Monoclonal antibodies that recognize p53, p63 or p73 can be labeled with the radioactive isotope ¹²⁵I using methods routinely practiced in the art.

In a heterogeneous assay, for example, the GST-ASP fusion protein can be anchored to glutathione-agarose beads. The p53, p63 or p73 protein preparation can then be added in the presence or absence of the test agent in a manner that allows interaction and binding to occur. At the end of the reaction period, unbound material can be washed away, and the labeled monoclonal antibody can be added to the system and allowed to bind to the complexed binding partners. The interaction between the ASP protein and the p53, p63 or p73 protein can be detected by measuring the amount of radioactivity that remains associated with the glutathione-agarose beads. A successful inhibition of the interaction by the test compound will result in a decrease in measured radioactivity. In contrast, increased stabilization of the interaction by the test compound will result in an increase in measured radioactivity.

Alternatively, the GST-ASP fusion protein and the p53, p63 or p73 protein can be mixed together in liquid in the absence of the solid glutathione agarose beads. The test agent can be added either during or after the binding partners are allowed to interact. This mixture can then be added to the glutathione-agarose beads and unbound material is washed away. Again, the extent of inhibition or stabilization of the binding partner interaction can be detected by adding the labeled antibody and measuring the radioactivity associated with the beads.

In another example, these same techniques can be employed using peptide fragments that correspond to the binding domains of the ASP protein and the p53, p63 or p73 protein, respectively, in place of one or both of the full length proteins. Any number of methods routinely practiced in the art can be used to identify and isolate the protein's binding site. These methods include, but are not limited to, mutagenesis of one of the genes encoding the proteins and screening for disruption of binding in a co-immunoprecipitation assay. Compensating mutations in a host gene can be selected. Sequence analysis of the genes encoding the respective proteins will reveal the mutations that correspond to the region of the protein involved in interactive binding. Alternatively, one protein can be anchored to a solid surface using methods described in above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the for the cellular or extracellular protein is obtained, short gene segments can be engineered to express peptide fragments of the protein, which can then be tested for binding activity and purified or synthesized.

For example, an ASP protein can be anchored to a solid material as described above by making a GST-ASP protein fusion protein and allowing it to bind to glutathione agarose beads. The p53, p63 or p73 protein can be labeled with a radioactive isotope, such as ³⁵S, and cleaved with a proteolytic enzyme such as trypsin. Cleavage products can then be added to the anchored GST-ASP protein fusion protein and allowed to bind. After washing away unbound peptides, labeled bound material, representing the cellular or extracellular protein binding domain, can be eluted, purified, and analyzed for amino acid sequence. Peptides so identified can be produced synthetically or fused to appropriate facilitative proteins using recombinant DNA technology.

### EXAMPLE 23

### Cell-Based Screening Assay for Agents that Modulate Apoptosis

This example describes methods using intact cells that can be used to screen test agents for their ability to modulate apoptosis. Similar to Example 22, therapeutic agents identified by these approaches are tested for their ability to increase or decrease apoptosis of a cell.

Generally, the method includes applying the test agent to a cell, wherein the cell expresses ASP (such as ASPP1 or ASPP2) along with p53, p63 or p73, and then determining whether the agent had an effect on apoptosis, determining whether the agent had an effect on Bax promoter activity, or determining if the agent increased expression of ASP. In particular examples, the amount of apoptosis, transactivation, or ASP expression/activity in the presence of the test agent is compared to an amount of apoptosis, transactivation, or ASP expression/activity in the absence of the test agent. In some examples, the test agent is applied to a cell growing in culture, such as an Saos-2 cell. In other examples, the method includes applying (or administering) the test agent to a tumor cell in vivo, such as a tumor expressing mutant p53 or expressing no p53 present in a mammal.

In particular examples, agents that decrease ASP expression or activity are selected for their potential to inhibit apoptosis (although 100% inhibition is not required, for example decreases of at least 20% could be considered inhibitory). Such agents can be further assayed for their ability to increase decrease apoptosis, for example using the assays provided in the Examples above. In other examples, agents that increase ASP expression or activity are selected for their potential to increase apoptosis. Such agents can be further assayed for their ability to increase apoptosis, for example using the assays provided in the Examples above.

Particular examples of an increase in apoptosis are increases of at least 20%, at least 50%, at least 100% or more, as compared to an amount of apoptosis in the absence of the therapeutic agent. Particular examples of a decrease in apoptosis are decreases of at least 20%, at least 50%, at least 90% or more, as compared to an amount of apoptosis in the absence of the therapeutic agent.

The amount of agent administered can be determined by skilled practitioners. In some examples, several different doses of the potential therapeutic agent can be administered to different cells or test subjects, to identify optimal dose ranges. In some examples, the test agent is administered in combination with another therapeutic agent (such as an anti-tumor agent), such as before, during, or after administering the test agent. Subsequent to the treatment, cells or tumors are observed for a change in apoptosis activity.

### EXAMPLE 24

### Rapid Screening Assays

Prior to performing assays to detect interference or stabilization with the association of an ASP protein and a p53, p63 or p73 protein, rapid screening assays can be used to screen a large number of agents to determine if they bind to the ASP or p53, p63 or p73 protein. Rapid screening assays for detecting binding to HIV proteins have been disclosed, for example in U.S. Pat. No. 5,230,998, which is incorporated by reference. For example, an ASP protein or a p53, p63 or p73 protein, is incubated with a first antibody capable of binding to the ASP, p53, p63 or p73 protein, and the agent to be screened. Excess unbound first antibody is washed and removed, and antibody bound to the ASP, p53, p63 or p73 protein is detected by adding a second labeled antibody that binds the first antibody. Excess unbound second antibody is then removed, and the amount of the label is quantitated. The effect of the binding effect is then determined in percentages by the formula: (quantity of the label in the absence of the test agent)−(quantity of the label in the presence of the test agent /quantity of the label in the absence of the test agent)×100.

Agents that are found to have a high binding affinity to the ASP, p53, p63 or p73 protein can then be used in other assays more specifically designed to test inhibition or enhancement of the ASP protein/p53, p63 or p73 protein interaction, or affect on apoptosis.

### EXAMPLE 25

### Recombinant Expression

With the disclosed sequences involved in apoptosis and Bax promoter activation, native and variant sequences can be generated. Expression and purification by standard laboratory techniques of any variant, such as a polymorphism, mutant, fragment or fusion of a sequence involved in apoptosis, such as SEQ ID NOS: 1-6, is enabled. One skilled in the art will understand that the sequences involved in apoptosis, as well as variants thereof, can be produced recombinantly in any cell or organism of interest, and purified prior to use.

Methods for producing recombinant proteins are well known in the art. Therefore, the scope of this disclosure includes recombinant expression of any disclosed protein, including variants, fusions and fragments thereof. For example, see U.S. Pat. No. 5,342,764 to Johnson et al.; U.S. Pat. No. 5,846,819 to Pausch et al.; U.S. Pat. No. 5,876,969 to Fleer et al. and Sambrook et al. (Molecular Cloning: A Laboratory Manual, Cold Spring Harbor, N.Y., 1989, Ch. 17, herein incorporated by reference).

Briefly, partial, full-length, or variant cDNA sequences of SEQ ID NOS: 1, 3 and 5 can be ligated into an expression vector, such as a bacterial expression vector. Proteins or peptides can be produced by placing a promoter upstream of the cDNA sequence. Examples of promoters include, but are not limited to lac, trp, tac, trc, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

Vectors suitable for the production of intact proteins include pKC30 (Shimatake and Rosenberg, 1981, Nature 292:128), pKK177-3 (Amann and Brosius, 1985, Gene 40:183) and pET-3 (Studiar and Moffatt, 1986, J. Mol. Biol. 189:113). A DNA sequence can be transferred to other cloning vehicles, such as other plasmids, bacteriophages, cosmids, animal viruses and yeast artificial chromosomes (YACs) (Burke et al., 1987, Science 236:806-12). These vectors can be introduced into a variety of hosts including somatic cells, and simple or complex organisms, such as bacteria, fungi (Timberlake and Marshall, 1989, Science 244:1313-7), invertebrates, plants (Gasser and Fraley, 1989, Science 244:1293), and mammals (Pursel et al., 1989, Science 244:1281-8), that are rendered transgenic by the introduction of the heterologous cDNA.

For expression in mammalian cells, a cDNA sequence, such as a coding sequence of any of SEQ ID NOS: 1, 3, or 5, can be ligated to heterologous promoters, such as the simian virus SV40, promoter in the pSV2 vector (Mulligan and Berg, 1981, Proc. Natl. Acad. Sci. USA 78:2072-6), and introduced into cells, such as monkey COS-1 cells (Gluzman, 1981, Cell 23:175-82), to achieve transient or long-term expression. The stable integration of the chimeric gene construct can be maintained in mammalian cells by biochemical selection, such as neomycin (Southern and Berg, 1982, J. Mol. Appl. Genet. 1:327-41) and mycophoenolic acid (Mulligan and Berg, 1981, Proc. Natl. Acad. Sci. USA 78:2072-6). Other exemplary vectors that can be used include, but are not limited to, pcDNA3.1 and pRc/CMV (Invitrogen, Carlsbad, Calif.) that contain a selectable marker such as a gene that confers G418 resistance (which facilitates the selection of stably transfected cell lines) and the human cytomegalovirus (CMV) enhancer-promoter sequences; pCEP4 vector (Invitrogen) which contains an Epstein Barr virus (EBV) origin of replication, facilitating the maintenance of plasmid as a multicopy extrachromosomal element; pEF-BOS plasmid containing the promoter of polypeptide Elongation Factor 1α, which stimulates efficiently transcription in vitro.

The transfer of DNA into eukaryotic, such as human or other mammalian cells is a conventional technique. The vectors are introduced into the recipient cells as pure DNA (transfection) by, for example, precipitation with calcium phosphate (Graham and vander Eb, 1973, Virology 52:466) strontium phosphate (Brash et al., 1987, Mol. Cell Biol. 7:2013), electroporation (Neumann et al., 1982, EMBO J. 1:841), lipofection (Felgner et al., 1987, Proc. Natl. Acad. Sci USA 84:7413), DEAE dextran (McCuthan et al., 1968, J. Natl. Cancer Inst. 41:351), microinjection (Mueller et al., 1978, Cell 15:579), protoplast fusion (Schafner, 1980, Proc. Natl. Acad. Sci. USA 77:2163-7), or pellet guns (Klein et al., 1987, Nature 327:70). Alternatively, the cDNA can be introduced by infection with virus vectors, for example retroviruses (Bernstein et al., 1985, Gen. Engrg. 7:235) such as adenoviruses (Ahmad et al., J. Virol. 57:267, 1986) or Herpes (Spaete et al., Cell 30:295, 1982).

### EXAMPLE 26

### Methods for in vivo or ex vivo Expression

The present disclosure provides methods of expressing ASPP1 or ASPP2, or functional equivalents thereof, in a cell or tissue in vivo. Such methods are useful if ASPP1 or ASPP2 activity is desired, such as for increasing apoptosis.

In one example, transfection of the cell or tissue occurs in vitro or ex vivo. In this example, the cell or tissue is removed from a subject and then transfected with an expression vector containing the desired cDNA (for example see U.S. Pat. No. 5,399,346). The transfected cells produce functional protein and can be reintroduced into the subject. In another example, a nucleic acid molecule is administered to the subject directly, and transfection occurs in vivo.

The scientific and medical procedures required for human cell transfection are now routine. The disclosure of ASPP1 or ASPP2 cDNA sequences allows the development of human (and other mammals) in vivo gene expression based upon these procedures. Immunotherapy of melanoma patients using genetically engineered tumor-infiltrating lymphocytes (TILs) has been reported by Rosenberg et al. (N. Engl. J. Med. 323:570-8, 1990), wherein a retrovirus vector was used to introduce a gene for neomycin resistance into TILs. A similar approach can be used to introduce ASPP1 or ASPP2 cDNA into subjects.

In some examples, a method of treating subjects in which greater ASPP1 or ASPP2 expression is desired is disclosed. These methods can be accomplished by introducing a gene coding for ASPP1 or ASPP2 into a subject. A general strategy for transferring genes into donor cells is disclosed in U.S. Pat. No. 5,529,774, incorporated by reference. Generally, a gene encoding a protein having therapeutically desired effects is cloned into a viral expression vector, and that vector is then introduced into the target organism. The virus infects the cells, and produces the protein sequence in vivo, where it has its desired therapeutic effect (Zabner et al. Cell 75:207-16, 1993). It may only be necessary to introduce the genetic or protein elements into certain cells or tissues, such as the cells of a tumor. However, in some instances, it may be more therapeutically effective and simple to treat all of a subject's cells, or more broadly disseminate the vector, for example by intravascular administration.

In particular examples, a nucleic acid sequence encoding ASPP1 or ASPP2 is under the control of a suitable promoter. Suitable promoters that can be employed include, but are not limited to, the gene's native promoter, retroviral LTR promoter, or adenoviral promoters, such as the adenoviral major late promoter; the CMV promoter; the RSV promoter; inducible promoters, such as the MMTV promoter; the metallothionein promoter; heat shock promoters; the albumin promoter; the histone promoter; the α-actin promoter; TK promoters; B19 parvovirus promoters; and the ApoAI promoter. However the scope of the disclosure is not limited to specific promoters.

The recombinant nucleic acid molecule can be administered to the subject by any method that allows the recombinant nucleic acid molecule to reach the appropriate cells. These methods include injection, infusion, deposition, implantation, or topical administration. Injections can be intradermal or subcutaneous. The recombinant nucleic acid molecule can be delivered as part of a viral vector, such as avipox viruses, recombinant vaccinia virus, replication-deficient adenovirus strains or poliovirus, or as a non-infectious form such as naked DNA or liposome encapsulated DNA, as further described in Example 27.

### EXAMPLE 27

### Viral Vectors for in vivo Gene Expression

Viral vectors can be used to express a desired ASPP1 or ASPP2 sequence in vivo. Methods for using such vectors for in vivo gene expression are well known (for example see U.S. Pat. No. 6,306,652 to Fallaux et al., U.S. Pat. No. 6,204,060 to Mehtali et al., U.S. Pat. No. 6,287,557 to Boursnell et al., and U.S. Pat. No. 6,217,860 to Woo et al., all herein incorporated by reference). Specific examples of such vectors include, but are not limited to: adenoviral vectors; adeno-associated viruses (AAV); retroviral vectors such as MMLV, spleen necrosis virus, RSV, Harvey Sarcoma Virus, avian leukosis virus, HIV, myeloproliferative sarcoma virus, and mammary tumor virus, as well as and vectors derived from these viruses. Other viral transfection systems may also be utilized, including Vaccinia virus (Moss et al., 1987, Annu. Rev. Immunol. 5:305-24), Bovine Papilloma virus (Rasmussen et al., 1987, Methods Enzymol. 139:642-54), and herpes viruses, such as Epstein-Barr virus (Margolskee et al., 1988, Mol. Cell. Biol. 8:2837-47). In another example, RNA-DNA hybrid oligonucleotides, as described by Cole-Strauss et al. (Science 273:1386-9, 1996) are used.

Viral particles are administered in an amount effective to produce a therapeutic effect in a subject. The exact dosage of viral particles to be administered is dependent upon a variety of factors, including the age, weight, and sex of the subject to be treated, and the nature and extent of the disease or disorder to be treated. The viral particles can be administered as part of a preparation having a titer of viral particles of at least 1×10¹⁰ pfu/ml, and in general not exceeding 2×10¹¹ pfu/ml. Viral particles can be administered in combination with a pharmaceutically acceptable carrier in a volume up to 10 ml. The pharmaceutically acceptable carrier may be, for example, a liquid carrier such as a saline solution, protamine sulfate (Elkins-Sinn, Inc., Cherry Hill, N.J.), or Polybrene (Sigma). Conventional pharmaceutically acceptable carriers are disclosed in Remington 's Pharmaceutical Sciences, by Martin, Mack Publishing Co., Easton, Pa., 15th Edition, 1975.

Having illustrated and described several uses of ASP and iASPP nucleic acid molecules, proteins, agonists, and antagonists, it should be apparent to one skilled in the art that the disclosure can be modified in arrangement and detail without departing from such principles. In view of the many possible embodiments to which the principles of our disclosure may be applied, it should be recognized that the illustrated embodiments are only particular examples of the disclosure and should not be taken as a limitation on the scope of the disclosure. Rather, the scope of the disclosure is in accord with the following claims. I therefore claim as my invention all that comes within the scope and spirit of these claims.

I claim:

1. A method of inducing apoptosis of a tumor cell, comprising:
contacting the cell with an agent having apoptosis stimulating protein of p53 (ASPP) biological activity, wherein the agent comprises an ASPP1 agent, or an ASPP2 agent, and wherein the ASPP1 agent or the ASPP2 agent induces apoptosis of the tumor cell.

2. The method of claim 1, wherein the ASPP1 agent or the ASPP2 agent comprises a mammalian ASPP1 agent or a mammalian ASPP2 agent.

3. The method of claim 2, wherein mammalian ASPP1 agent or mammalian ASPP2 agent comprises a human ASPP1 agent or a human ASPP2 agent.

4. The method of claim 1, wherein the ASPP1 agent comprises an ASPP1 protein having at least 90% sequence identity to SEQ ID NO: 2.

5. The method of claim 1, wherein the ASPP1 agent is an ASPP1 nucleic acid molecule that encodes a protein having ASP biological activity.

6. The method of claim 5, wherein the ASPP1 nucleic acid molecule comprises a sequence having at least 90% sequence identity to SEQ ID NO: 1.

7. The method of claim 1, wherein the ASPP2 agent comprises an ASPP2 protein having at least 90% sequence identity to SEQ ID NO: 4.

8. The method of claim 1, wherein the ASPP2 agent is an ASPP2 nucleic acid molecule that encodes a protein having ASP biological activity.

9. The method of claim 8, wherein the ASPP2 nucleic acid molecule comprises a sequence having at least 90% sequence identity to SEQ ID NO: 3.

10. The method of claim 1, wherein the tumor cell expresses p63 or p73.

11. The method of claim 1, wherein the tumor cell does not express functional p53.

12. The method of claim 1, wherein the tumor expresses a mutant p53 protein.

13. The method of claim 1, wherein the tumor cell is present in a subject having a tumor, and wherein contacting the tumor cell with the agent comprises administering the agent to the subject.

14. The method of claim 13, wherein inducing apoptosis of the tumor cell reduces a volume of the tumor by at least 10%.

15. The method of claim 13, wherein inducing apoptosis of the tumor cell reduces metastasis of the tumor by at least 10%.

16. The method of claim 13, wherein an amount of p53, p63, or p73 expression in the tumor is determined prior to administering the agent to the subject.

17. The method of claim 13, wherein the subject is a human.

18. The method of claim 16, wherein determining an amount of p53 expression comprises determining an amount of p53 activity in the tumor.

19. The method of claim 13, further comprising administering a chemotherapeutic agent to the subject.

20. A method of inducing apoptosis of a tumor cell, comprising:
increasing ASPP1 or ASPP2 expression or activity in a cell, wherein the ASPP1 or ASPP2 expression or activity induces apoptosis of the tumor cell.

21. The method of claim 20, wherein increasing ASPP1 or ASPP2 expression comprises administering a nucleic acid molecule encoding an ASPP1 or ASPP2 protein to the cell.

22. The method of claim 20, wherein increasing ASPP1 or ASPP2 activity comprises administering an ASPP1 or ASPP2 protein to the cell.

23. The method of claim 22, wherein the tumor cell is present in a subject having a tumor that expresses p63 or p73, and wherein administering the ASPP1 or ASPP2 protein to the cell comprises administering the ASPP1 or ASPP2 protein to the subject.

24. The method of claim 23, wherein an amount of p53, p63, or p73 expression in the tumor is determined prior to increasing ASPP1 or ASPP2 expression or activity in a cell.

25. The method of claim 23, wherein the tumor cell does not express functional p53.

26. The method of claim 23, wherein the tumor expresses a mutant p53 protein.

27. A method of modulating p63 apoptotic activity or p73 apoptotic activity in a cell, comprising:
modulating ASPP1 or ASPP2 expression or activity in a cell, wherein the ASPP1 or ASPP2 expression or activity modulates p63 apoptotic activity or p73 apoptotic activity apoptosis in the cell.

28. The method of claim 27, wherein the method is a method of increasing p63 apoptotic activity or p73 apoptotic activity in the cell, and modulating ASPP1 or ASPP2 expression or activity in a cell comprises increasing ASPP1 or ASPP2 expression or activity in a cell.

29. The method of claim 28, wherein the cell is a tumor cell, and increasing p63 activity or p73 activity induces apoptosis of the tumor cell.

30. The method of claim 27, wherein p63 apoptotic activity or p73 apoptotic activity comprises Bax promoter activity.

31. The method of claim 27, wherein the method is a method of decreasing p63 activity or p73 activity in the cell, and modulating ASPP1 or ASPP2 expression or activity in a cell comprises decreasing ASPP1 or ASPP2 expression or activity in a cell.

32. The method of claim 31, wherein the cell is a tumor cell that overexpresses p63 or p73.

33. The method of claim 32, wherein the tumor cell that overexpresses p73 is a neuroblastoma cells, hepatocellular carcinoma cell, colorectal cancer cell, breast cancer cell, or liver cholangiocarcinoma cell.

34. A method of treating a p63 mediated condition or a p73 mediated condition in a subject, comprising:
modulating ASPP1 or ASPP2 expression or activity in the subject, wherein the ASPP1 or ASPP2 expression or activity treats the p63 mediated condition or the p73 mediated condition in a subject.

35. The method of claim 34, wherein the p63 mediated condition is Ectrodactyly, Ectodermal dysplasia and facial Clefts (EEC) and modulating ASPP1 or ASPP2 expression or activity in the subject comprises increasing ASPP1 or ASPP2 expression or activity in the subject.

36. The method of claim 35, wherein the p73 mediated condition is neuroblastoma or T-cell lymphoma and modulating ASPP1 or ASPP2 expression or activity in the subject comprises increasing ASPP1 or ASPP2 expression or activity in the subject.

37. A method of identifying an agent that modulates apoptosis, comprising:
contacting an ASP protein and a p53, p63 or p73 protein with a test agent; and
determining whether binding of the ASP protein to the p53, p63 or p73 protein is changed in the presence of the test agent, wherein a decrease in binding being an indication that the test agent decreases the binding of ASP protein to the p53, p63 or p73 protein, and decreases apoptosis, and wherein an increase in binding being an indication that the test agent increases the binding of ASP protein to the p53, p63 or p73 protein, and increases apoptosis.

38. The method of claim 37, wherein the method comprises expressing the ASP protein and the p53, p63 or p73 protein in a cell, and contacting the ASP protein and the p53, p63 or p73 protein with the test agent comprises exposing the cell to the test agent.

39. The method of claim 37, wherein the host protein or the p53, p63 or p73 protein comprises a label, and determining whether binding is decreased comprises detecting an amount of label present.

40. A method of identifying an agent that modulates apoptosis, comprising:
contacting a cell with a test agent, wherein the cell expresses an ASP protein and a p53, p63 or p73 protein; and
determining whether the cell undergoes apoptosis, wherein a decrease in apoptosis being an indication that the test agent decreases apoptosis, and wherein an increase in apoptosis being an indication that the test agent increases apoptosis.

41. The method of claim 40, further comprising determining an amount of Bax promoter activity, wherein a decrease in Bax promoter activity being an indication that the test agent decreases apoptosis, and wherein an increase in Bax promoter activity being an indication that the test agent increases apoptosis.


SEQUENCE LISTING (10 sequences)

SEQ ID NO: 1 - DNA, 4834 bp, Homo sapiens
  CDS (159)..(3431)
gagccccgcatcccgccgcagctgccgcctcgccgcggccgggccggagagcacggcggcgggagcgcggccttaggaggcggccggagcggtgggcacagctcggcgcggagcgtcctgtcaggcggcggccgagggcgtcgcggactctccccgcgatgatgccgatgatattaactgttttcttgagcaacaatgaacagattttaacagaagttcctataacaccggaaacaacctgtcgagatgttgtagaattttgcaaggaacctggagaaggcagctgccatttagctgaagtgtggaggggaaatgaacgtcccataccctttgatcatatgatgtacgaacatcttcagatatggggtccacggagggaagaagtgaaatttttccttcgacacgaggactccccaactgagaacagtgaacaaggtggccgtcagacccaagagcaacgaactcagagaaatgtaataaatgtacctggagataaacgtactgaatatggggttgggaatccacgtgttgaacttaccctctcagagctccaagatatggcagctaggcaacagcagcagattgaaaatcagcagcagatgttggttgccaaggaacagcgtttacattttctaaagcaacaggagcgccgtcagcagcagtctatttctgaaaatgaaaagcttcagaaattgaaagaacgagttgaagcccaggagaacaagctgaagaaaattcgtgcaatgagaggacaagtcgactacagcaaaatcatgaacggcaatctgtctgctgaaatagaaaggttcagtgccatgttccaggaaaagaagcaggaagtacagactgcaattttaagggttgatcagcttagtcagcaattggaagatttaaagaaaggaaaactgaatgggttccagtcttacaatggcaaattgacgggaccagcggcggtggagttaaaaagactgtaccaagaactacagattcgtaaccaacttaaccaggaacaaaattcaaaacttcagcagcagaaggaactcttaaataagcgcaacatggaggtggccatgatggacaagcgaatcagtgaactgcgtgaacgtctctatgggaaaaaaattcagctgaaccgtgtgaatggcacgtcatcaccacagtcccctctgagcacatcgggcagggtcgctgctgtggggccttatatccaggttcccagtgccggaagctttcctgtgctgggggaccctataaagccccagtctctcagtattgcctcaaatgctgctcatggaagatccaaatccgctaatgatggaaactggccaacattaaaacagaattctagctcttccgtgaaaccagtgcaggtggccggtgcagactggaaggatccgagcgtggaggggtctgtcaagcagggcactgtctccagccagcctgtgcccttctcagcactgggacccacggagaagccgggcatcgagattggtaaagtgccacctcccatcccgggtgtaggcaagcagctgcctccaagctatgggacatacccaagtcctacacctctgggtcctgggtcgacaagctccctggaaaggaggaaggaaggcagcttgcccaggcccagtgcaggcctgccaagtcgacagaggcccaccctgctgcccgccacaggcagcaccccccagccaggctcctcacaacagattcagcagaggatttccgtaccgccaagtcccacgtacccgccagcgggaccacctgcatttccagctggggacagcaagcctgaactcccactgacagtggccattaggcctttcctggctgataaagggtcaaggccacagtctcccaggaaaggaccccagacagtgaattcaagttccatatactccatgtacctccagcaagccacaccacctaagaattaccagccggcagcacacagcgccttaaataagtcagttaaagcagtgtatggtaagcccgttttaccttcgggttcaacctctccatcgccgctgccgtttcttcacgggtcactgtccacgggcacaccacagcctcagccaccttcagaaagtactgagaaagagcctgagcaggatggccccgccgcccccgcagatggcagcaccgtggagagcctgccacggccactcagccccaccaagctcacgcccatcgtgcattcgccactgcgctaccagagtgatgcagacctggaggccctccgcaggaagctggccaacgcgccccggcccctgaaaaagcgcagctccatcacagagcccgagggccccggcgggcccaacatccagaagctgctgtaccagcgcttcaacaccctggccggtggcatggagggcacccctttctaccagcccagcccctcccaggacttcatgggcaccttggccgatgtggacaatggaaacaccaatgccaatggaaacctggaagagctcccccctgcccagcccacagccccactccccgctgagcctgccccgtcatcagatgccaatgataatgagttaccttcccccgaaccagaggagctcatctgtccccaaaccacccaccaaactgccgagccggcagaggacaataacaacaacgtggccacggtccccaccacggagcagatcccgagtcctgtggctgaggccccatctccaggggaagagcaggtccctccagcacctcttccccctgccagccaccctcctgccacctccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgatgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcctgaacttccttttggagcaccgcatggtcttgccagctaccaggagccacttaagagattattgtgctgttttccaggaaagctgcagctagaaaatggtcttaatggtgctcactttagcagacagcgtccacaatgtgaatcctacagtttccaggtgaggccctttctccagtttgcccattaactgggagaggtactttcgcctccaaggactgaattttgccaattactataaatccaaataaatacccactttcaaaacacccacccctcttgccattaagaagtcccataacccccggttggttgccagtgaagacagaagctcttactgacttggccccgaggccatcaccccctccagcagtgaacactgtccgccgctgtgaggcctgctcccctgcgaccgccctgccccccgtcaccgaatcggacactcatcctttctcacacttcccacacatgatccttcttcccttcatcaccaaaggagcctctgtatggaaacatgtccagtgttgctgcccagtgtgtatgcctcccagtacccactctgctcggccgccttgggggttccgcttcctgttccagttcacctaaaggctgattgtgcaggcccagcactgtggctggactgccgcgccacgggcaccaggacccctaagaccaagtgacaactgggagagcctcagcatatactcttctcctccgatctcacagcctgtcatgctgctcagtgtggttctcacccctgcaagctcaaattcagttccctgaatggagtcaggtgctggaggccgtggcagcggagggtggttggggttggggctgggggtggactggtgtgagggcagaccagggccaggtagacggggctgtttggtgcctgaaggatggcagacgcctggtgtcaggaggggccgccaccaaggagcagcagctggggcagaggagctggggtcaggggccacccctctctgccgatctccctgcctgggctggctgtgaggccacctttgtcccaggcccagcctcaaggcaaggagggcgcttcactgaggtgtgaattgtacgtacaggctttttatataccaaaagtattttttgactagaccattcaaagctacccgaactatgttggaaatttttttttttctcattaaaatacaggcccttaggctctatttttcatgtatgagtcgtgtgtaatttatgtaaaaatgtgtgtacagactcactgatgcagcactgtagcccatcaccttggagcactgactgtacatagtgtggtgaagaaaagtgaacgcccttgtagagcagcccgaccacaggagcatggccgctgccagcccagacgctgctgacgctgtgtaaatgtgcacaataaacccgtctcaccccgg

SEQ ID NO: 2 - protein, 1090 aa, Homo sapiens
MMPMILTVFLSNNEQILTEVPITPETTCRDVVEFCKEPGEGSCHLAEVWRGNERPIPFDHMMYEHLQIWGPRREEVKFFLRHEDSPTENSEQGGRQTQEQRTQRNVINVPGDKRTEYGVGNPRVELTLSELQDMAARQQQQIENQQQMLVAKEQRLHFLKQQERRQQQSISENEKLQKLKERVEAQENKLKKIRAMRGQVDYSKIMNGNLSAEIERFSAMFQEKKQEVQTAILRVDQLSQQLEDLKKGKLNGFQSYNGKLTGPAAVELKRLYQELQIRNQLNQEQNSKLQQQKELLNKRNMEVAMMDKRISELRERLYGKKIQLNRVNGTSSPQSPLSTSGRVAAVGPYIQVPSAGSFPVLGDPIKPQSLSIASNAAHGRSKSANDGNWPTLKQNSSSSVKPVQVAGADWKDPSVEGSVKQGTVSSQPVPFSALGPTEKPGIEIGKVPPPIPGVGKQLPPSYGTYPSPTPLGPGSTSSLERRKEGSLPRPSAGLPSRQRPTLLPATGSTPQPGSSQQIQQRISVPPSPTYPPAGPPAFPAGDSKPELPLTVAIRPFLADKGSRPQSPRKGPQTVNSSSIYSMYLQQATPPKNYQPAAHSALNKSVKAVYGKPVLPSGSTSPSPLPFLHGSLSTGTPQPQPPSESTEKEPEQDGPAAPADGSTVESLPRPLSPTKLTPIVHSPLRYQSDADLEALRRKLANAPRPLKKRSSITEPEGPGGPNIQKLLYQRFNTLAGGMEGTPFYQPSPSQDFMGTLADVDNGNTNANGNLEELPPAQPTAPLPAEPAPSSDANDNELPSPEPEELICPQTTHQTAEPAEDNNNNVATVPTTEQIPSPVAEAPSPGEEQVPPAPLPPASHPPATSTNKRTNLKKPNSERTGHGLRVRFNPLALLLDASLEGEFDLVQRIIYEVEDPSKPNDEGITPLHNAVCAGHHHIVKFLLDFGVNVNAADSDGWTPLHCAASCNSVHLCKQLVESGAAIFASTISDIETAADKCEEMEEGYIQCSQFLYGVQEKLGVMNKGVAYALWDYEAQNSDELSFHEGDALTILRRKDESETEWWWARLGDREGYVPKNLLGLYPRIKPRQRTLA

SEQ ID NO: 3 - DNA, 4402 bp, Homo sapiens
  CDS (256)..(3642)
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcgtctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctccccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 4 - protein, 1128 aa, Homo sapiens
MMPMFLTVYLSNNEQHFTEVPVTPETICRDVVDLCKEPGESDCHLAEVWCGSERPVADNERMFDVLQRFGSQRNEVRFFLRHERPPGRDIVSGPRSQDPSLKRNGVKVPGEYRRKENGVNSPRMDLTLAELQEMASRQQQQIEAQQQLLATKEQRLKFLKQQDQRQQQQVAEQEKLKRLKEIAENQEAKLKKVRALKGHVEQKRLSNGKLVEEIEQMNNLFQQKQRELVLAVSKVEELTRQLEMLKNGRIDSHHDNQSAVAELDRLYKELQLRNKLNQEQNAKLQQQRECLNKRNSEVAVMDKRVNELRDRLWKKKAALQQKENLPVSSDGNLPQQAASAPSRVAAVGPYIQSSTMPRMPSRPELLVKPALPDGSLVIQASEGPMKIQTLPNMRSGAASQTKGSKIHPVGPDWSPSNADLFPSQGSASVPQSTGNALDQVDDGEVPLREKEKKVRPFSMFDAVDQSNAPPSFGTLRKNQSSEDILRDAQVANKNVAKVPPPVPTKPKQINLPYFGQTNQPPSDIKPDGSSQQLSTVVPSMGTKPKPAGQQPRVLLSPSIPSVGQDQTLSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPEEPNPEAPHVLDVYLEEYPPYPPPPYPSGEPEGPGEDSVSMRPPEITGQVSLPPGKRTNLRKTGSERIAHGMRVKFNPLALLLDSSLEGEFDLVQRIIYEVDDPSLPNDEGITALHNAVCAGHTEIVKFLVQFGVNVNAADSDGWTPLHCAASCNNVQVCKFLVESGAAVFAMTYSDMQTAADKCEEMEEGYTQCSQFLYGVQEKMGIMNKGVIYALWDYEPQNDDELPMKEGDCMTIIHREDEDEIEWWWARLNDKEGYVPRNLLGLYPRIKPRQRSLA

SEQ ID NO: 5 - DNA, 1056 bp, homo sapiens
  CDS (1)..(1056)
atgtggatgaaggaccctgtagcaaggcctctcagccccacgaggctgcagccagcactgccaccggaggcacagtcggtgcccgagctggaggaggtggcacgggtgttggcggaaattccccggcccctcaaacgcaggggctccatggagcaggcccctgctgtggccctgccccctacccacaagaaacagtaccagcagatcatcagccgcctcttccatcgtcatggggggccagggcccggggggcggagccagagctgtcccccatcactgagggatctgaggccagggcagggccccctgctcctgccccaccagctcccattccaccgcccggccccgtcccagagcagcccaccagagcagccgcagagcatggagatgcgctctgtgctgcggaaggcgggctccccgcgcaaggcccgccgcgcgcgcctcaaccctctggtgctcctcctggacgcggcgctgaccggggagctggaggtggtgcagcaggcggtgaaggagatgaacgacccgagccagcccaacgaggagggcatcactgccttgcacaacgccatctgcggcgccaactactctatcgtggatttcctcatcaccgcgggtgccaatgtcaactcccccgacagccacggctggacacccttgcactgcgcggcgtcgtgcaacgacacagtcatctgcatggcgctggtgcagcacggcgctgcaatcttcgccaccacgctcagcgacggcgccaccgccttcgagaagtgcgacccttaccgcgagggttatgctgactgcgccacctacctggcagacgtcgagcagagtatggggctgatgaacagcggggcagtgtacgctctctgggactacagcgccgagttcggggacgagctgtccttccgcgagggcgagtcggtcaccgtgctgcggagggacgggccggaggagaccgactggtggtgggccgcgctgcacggccaggagggctacgtgccgcggaactacttcgggctgttccccagggtgaagcctcaaaggagtaaagtctag

SEQ ID NO: 6 - protein, 351 aa, homo sapiens
MWMKDPVARPLSPTRLQPALPPEAQSVPELEEVARVLAEIPRPLKRRGSMEQAPAVALPPTHKKQYQQIISRLFHRHGGPGPGGRSQSCPPSLRDLRPGQGPLLLPHQLPFHRPAPSQSSPPEQPQSMEMRSVLRKAGSPRKARRARLNPLVLLLDAALTGELEVVQQAVKEMNDPSQPNEEGITALHNAICGANYSIVDFLITAGANVNSPDSHGWTPLHCAASCNDTVICMALVQHGAAIFATTLSDGATAFEKCDPYREGYADCATYLADVEQSMGLMNSGAVYALWDYSAEFGDELSFREGESVTVLRRDGPEETDWWWAALHGQEGYVPRNYFGLFPRVKPQRSKV

SEQ ID NO: 7 - DNA, 66 bp, Artificial
  p63 sense oligonucleotide
gatcccctgaattcctcagtccagaggttcaagagacctctggactgaggaattcatttttggaaa

SEQ ID NO: 8 - DNA, 66 bp, Artificial
  p63 antisense oligonucleotide
agcttttccaaaaatgaattcctcagtccagaggtctcttgaacctctggactgaggaattcaggg

SEQ ID NO: 9 - DNA, 63 bp, Artificial
  p73 sense oligonucleotide
gatccccgccgggggaataatgaggtttcaagagaacctcattattcccccggcttttggaaa

SEQ ID NO: 10 - DNA, 64 bp, Artificial
  p73 antisense oligonucleotide
agcttttccaaaaagccgggggaataatgaggttctcttgaaacctcattattcccccggcggg